(19) United States
(12) Patent Application Publication
(10) Pub. No.: US 2025/0164877 A1
(43) Pub. Date: May 22, 2025

(54) RADIATION-SENSITIVE COMPOSITION, METHOD FOR FORMING RESIST PATTERN, AND RADIATION-SENSITIVE ACID GENERATOR

(71) Applicant: JSR CORPORATION, Tokyo (JP)

(72) Inventors: Ryuichi NEMOTO, Tokyo (JP); Masayuki MIYAKE, Tokyo (JP); Michihiro MITA, Tokyo (JP); Kensuke MIYAO, Tokyo (JP); Satoshi OKAZAKI, Tokyo (JP)

(73) Assignee: JSR CORPORATION, Tokyo (JP)

(21) Appl. No.: 19/034,826

(22) Filed: Jan. 23, 2025

Related U.S. Application Data
(63) Continuation of application No. PCT/JP2023/027258, filed on Jul. 25, 2023.

(30) Foreign Application Priority Data
Jul. 26, 2022 (JP) 2022-119054

(51) Int. Cl. G03F 7/004; G03F 7/039; G03F 7/32

(52) U.S. Cl. CPC G03F 7/0045; G03F 7/0392; G03F 7/322

(57) ABSTRACT

A radiation-sensitive composition contains a polymer having an acid-releasable group, and a compound represented by formula (1). In the formula (1), L¹ represents a group having a (thio)acetal ring or the like. W¹ represents a single bond or a (b+1)-valent organic group having 1 to 40 carbon atoms. R¹, R², and R³ each independently represent a hydrogen atom, a hydrocarbon group having 1 to 10 carbon atoms, a fluorine atom, or a fluoroalkyl group. R^{f} represents a fluorine atom or a fluoroalkyl group. a represents an integer of 0 to 8. b represents an integer of 1 to 4. d represents 1 or 2. When a represents 2 or more, a plurality of R¹ are the same or different, and a plurality of R² are the same or different. M⁺ represents a monovalent cation.

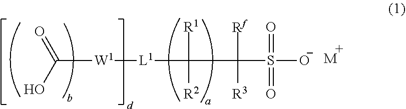

### CROSS-REFERENCE TO RELATED APPLICATIONS

The present application is a continuation application of International Patent Application No. PCT/JP2023/027258 filed Jul. 25, 2023, which claims priority based on Japanese Patent Application No. 2022-119054, filed on Jul. 26, 2022. The contents of these applications are incorporated herein by reference in their entirety.

### BACKGROUND OF THE DISCLOSURE

### Technical Field

The present disclosure relates to a radiation-sensitive composition, a method for forming a resist pattern, and a radiation-sensitive acid generator.

### Discussion of the Background

For forming fine circuits in semiconductor elements, photolithography technique using a radiation-sensitive composition is used. As a representative procedure of the photolithography technology, a coating formed with the radiation-sensitive composition (hereinafter, also referred to as “resist film”) is firstly irradiated with radiation thorough a mask pattern, and a chemical reaction with an acid generated by the irradiation with radiation causes a difference in a dissolution rate in a developer liquid between an exposed portion and an unexposed portion in the resist film. Then, the resist film after the exposure is contacted with the developer liquid to dissolve the exposed portion or the unexposed portion in the developer liquid. This procedure forms a resist pattern on a substrate.

For forming circuits of a semiconductor element by the photolithography technique, various investigations on a radiation-sensitive acid generator, which is one of main components in the radiation-sensitive composition, have been in progress in order to form a finer resist pattern (for example, see Japanese Patent Laid-Open No. 2011-37837 and Japanese Patent Laid-Open No. 2018-135321). Japanese Patent Laid-Open No. 2011-37837 discloses a radiation-sensitive composition containing, as an acid generator, a salt composed of: an anion having a spiro-ring structure of a (thio)acetal ring and a saturated ring; and a cation. Japanese Patent Laid-Open No. 2018-135321 discloses a radiation-sensitive composition containing, as an acid generator, a salt composed of: an anion having a spiro-ring structure of a (thio)acetal lactone ring and an alicyclic hydrocarbon; and a cation.

### SUMMARY

According to an aspect of the present disclosure, a radiation-sensitive composition includes a polymer having an acid-releasable group and a compound represented by formula (1).

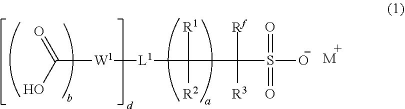

L¹ represents a group having a (thio)acetal ring formed by replacing each of two methylene groups of a monocyclic saturated aliphatic hydrocarbon ring by a (thio)ether bond so that two oxygens, two sulfurs, or one oxygen and one sulfur are bonded to one and the same carbon, or represents a group represented by formula (L-2):

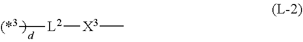

wherein L² represents a bridged alicyclic group having 7 or more carbon atoms; X³ represents a single bond, an oxygen atom, a sulfur atom, or —SO₂—; “d” represents 1 or 2; and “*³” represents a chemical bond to W¹ or a carboxy group,
W¹ represents a single bond or a (b+1)-valent organic group having 1 to 40 carbon atoms; R¹, R², and R³ each independently represent a hydrogen atom, a hydrocarbon group having 1 to 10 carbon atoms, a fluorine atom, or a fluoroalkyl group; R^{f} represents a fluorine atom or a fluoroalkyl group; “a” represents an integer of 0 to 8; “b” represents an integer of 1 to 4; “d” represents 1 or 2; when L¹ represents the group represented by the formula (L-2), “d” in the formula (1) and “d” in the formula (L-2) are the same values; when “a” represents 2 or more, a plurality of R¹ are the same or different, and a plurality of R² are the same or different; when “d” represents 2, a plurality of W¹ are the same or different, and a plurality of “b” are the same or different; and M⁺ represents a monovalent cation.

According to another aspect of the present disclosure, a method for forming a resist pattern includes applying the above radiation-sensitive composition on a substrate to form a resist film, exposing the resist film, and developing the exposed resist film.

According to a further aspect of the present disclosure, the radiation-sensitive acid generator is represented by formula (1)

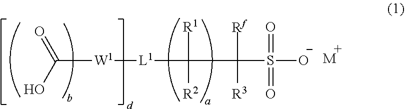

L¹ represents a group having a (thio)acetal ring formed by replacing each of two methylene groups of a monocyclic saturated aliphatic hydrocarbon ring by a (thio)ether bond so that two oxygens, two sulfurs, or one oxygen and one sulfur are bonded to one and the same carbon, or represents a group represented by formula (L-2):

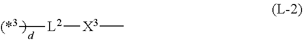

wherein L² represents a bridged alicyclic group having 7 or more carbon atoms; X³ represents a single bond, an oxygen atom, a sulfur atom, or —SO₂—; “d” represents 1 or 2; and “*³” represents a chemical bond to W¹ or a carboxy group,
W¹ represents a single bond or a (b+1)-valent organic group having 1 to 40 carbon atoms; R¹, R², and R³ each independently represent a hydrogen atom, a hydrocarbon group having 1 to 10 carbon atoms, a fluorine atom, or a fluoroalkyl group; R^{f} represents a fluorine atom or a fluoroalkyl group; “a” represents an integer of 0 to 8; “b” represents an integer of 1 to 4; “d” represents 1 or 2; when L¹ represents the group represented by the formula (L-2), “d” in the formula (1) and “d” in the formula (L-2) are the same values; when “a” represents 2 or more, a plurality of R¹ are the same or different, and a plurality of R² are the same or different; when “d” represents 2, a plurality of W¹ are the same or different, and a plurality of “b” are the same or different; and M⁺ represents a monovalent cation.

### DESCRIPTION OF THE EMBODIMENTS

As used herein, the words “a” and “an” and the like carry the meaning of “one or more.”

When an amount, concentration, or other value or parameter is given as a range, and/or its description includes a list of upper and lower values, this is to be understood as specifically disclosing all integers and fractions within the given range, and all ranges formed from any pair of any upper and lower values, regardless of whether subranges are separately disclosed. Where a range of numerical values is recited herein, unless otherwise stated, the range is intended to include the endpoints thereof, as well as all integers and fractions within the range. As an example, a stated range of 1-10 fully describes and includes the independent subrange 3.4-7.2 as does the following list of values: 1, 4, 6, 10.

The photolithography technique using the radiation-sensitive composition has achieved a finer pattern by utilizing radiation with a short wavelength, such as ArF excimer laser, and by using liquid-immersion lithography in which the exposure is performed in a state where a space between a lens of an exposure apparatus and the resist film is filled with a liquid medium. As next-generation technique, lithography technique using radiation with a shorter wavelength, such as electron beam, X-ray, and extreme ultraviolet ray (EUV), have been investigated. In these efforts toward the next-generation technique, required are performance higher than conventional performance in term of radiation sensitivity of the radiation-sensitive composition, line width roughness (LWR) performance, which is an index indicating unevenness of line width of the resist pattern, profile of the resist pattern (for example, rectangularity of a sectional shape of the resist pattern, etc.) and reduction in development defects.

The radiation-sensitive composition of the present disclosure contains the polymer having an acid-releasable group and the compound represented by the formula (1), and can consequently exhibit excellent LWR performance and pattern profile in resist pattern formation while exhibiting high sensitivity and can reduce development defects. Since the method for forming a resist pattern of the present disclosure uses the radiation-sensitive composition of the present disclosure, a resist pattern with excellent LWR performance and pattern profile and reduced development defects can be obtained. Therefore, the fine resist pattern can have further higher accuracy and higher performance. In addition, the radiation-sensitive acid generator of the present disclosure exhibits high sensitivity, and can form the resist pattern that can exhibit excellent LWR performance and pattern profile in resist pattern formation, and with reduced development defects.

Hereinafter, items relating to embodiments of the present disclosure will be described in detail. Note that a numerical range described by using “to” herein has a meaning including values described before and after “to” as a lower limit and an upper limit.

### <<Radiation-Sensitive Composition>>

The radiation-sensitive composition of the present disclosure (“hereinafter, also referred to as “the present composition”) contains a polymer having an acid-releasable group (hereinafter, also referred to as “polymer (A)”) and a compound having a specific anion structure (hereinafter, also referred to as “compound (B)”). The present composition may contain other optional components within a range not impairing the effect of the present disclosure. Hereinafter, each component will be described in detail.

Note that “hydrocarbon group” herein has a meaning including a chain hydrocarbon group, an alicyclic hydrocarbon group, and an aromatic hydrocarbon group. The term “chain hydrocarbon group” means a linear hydrocarbon group and a branched hydrocarbon group that have no cyclic structure and that are constituted with only a chain structure. It is to be noted that the chain hydrocarbon group may be saturated or unsaturated. The term “alicyclic hydrocarbon group” means a hydrocarbon group that has only an alicyclic hydrocarbon structure as a cyclic structure and that has no aromatic cyclic structure. The alicyclic hydrocarbon group is not necessarily constituted with only the alicyclic hydrocarbon structure, and includes a group having a chain structure in a part thereof. The term “aromatic hydrocarbon group” means a hydrocarbon group having an aromatic cyclic structure as a cyclic structure. It is to be noted that the aromatic hydrocarbon group is not necessarily constituted with only the aromatic cyclic structure, and may have a chain structure or an alicyclic hydrocarbon structure in a part thereof. The term “organic group” means an atomic group in which any hydrogen atom is removed from a compound containing carbon (namely organic compound). The term “(meth)acryl” encompasses “acryl” and “methacryl”. The term “(thio)ether” encompasses “ether” and “thioether”. The term “(thio)acetal” encompasses “acetal” and “thioacetal”.

The description “substituted or unsubstituted p-valent hydrocarbon group, wherein “p” represents an integer of 1 or more” encompasses a p-valent hydrocarbon group (namely, unsubstituted p-valent hydrocarbon group) and a group in which “p” hydrogen atoms are removed from a hydrocarbon structure moiety in a hydrocarbon group having a substituent. As an example of the substituted or unsubstituted p-valent hydrocarbon group, for example, an alkyl group and a fluoroalkyl group correspond to a case of p=1, and an alkanediyl group and a fluoroalkanediyl group correspond to a case of p=2. Among these, the fluoroalkyl group corresponds to “substituted monovalent hydrocarbon group”, and the fluoroalkanediyl group corresponds to “substituted divalent hydrocarbon group”. The same applies to other groups described with “substituted or unsubstituted”.

### <Polymer (A)>

The acid-releasable group in the polymer (A) is a group that substitutes a hydrogen atom in an acid group (for example, a carboxy group, a phenolic hydroxy group, an alcoholic hydroxy group, and a sulfo group), and is a group releasable by an action of an acid. By blending the polymer having the acid-releasable group in the radiation-sensitive composition, the acid-releasable group is dissociated to generate the acid group by a chemical reaction involving an acid generated by irradiating the radiation-sensitive composition with radiation, which can change solubility of the polymer in a developer liquid. As a result, good lithography properties can be imparted to the present composition.

The polymer (A) preferably has a structural unit having an acid-releasable group (hereinafter, also referred to as “structural unit (I)”). Examples of the structural unit (I) include a structural unit having a structure in which a hydrogen atom in a carboxy group is replaced by a substituted or unsubstituted tertiary hydrocarbon group, a structural unit having a structure in which a hydrogen atom in a phenolic hydroxy group is replaced by a substituted or unsubstituted tertiary hydrocarbon group, and a structural unit having an acetal structure. Among these, from the viewpoint of improving pattern profile of the present composition, the structural unit (I) is preferably the structural unit having a structure in which a hydrogen atom in a carboxy group is replaced by a substituted or unsubstituted tertiary hydrocarbon group, and specifically preferably a structural unit represented by the following formula (2) (hereinafter, referred to as “structural unit (I-1)”).

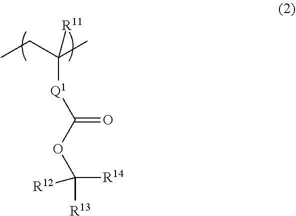

In the formula (2), R¹¹ represents a hydrogen atom, a fluorine atom, a methyl group, a trifluoromethyl group, or an alkoxyalkyl group. Q¹ represents a single bond or a substituted or unsubstituted divalent hydrocarbon group. R¹² represents a substituted or unsubstituted monovalent hydrocarbon group having 1 to 20 carbon atoms. R¹³ and R¹⁴ each independently represent a monovalent chain hydrocarbon group having 1 to 10 carbon atoms or a monovalent alicyclic hydrocarbon group having 3 to 20 carbon atoms, or a divalent alicyclic hydrocarbon group having 3 to 20 carbon atoms constituted by combining R¹³ and R¹⁴ together with the carbon atom to which R¹³ and R¹⁴ are bonded.

In the formula (2), R¹¹ preferably represents a hydrogen atom or a methyl group, and more preferably a methyl group from the viewpoint of copolymerization properties of a monomer to yield the structural unit (I-1). The divalent hydrocarbon group represented by Q¹ is preferably a divalent aromatic group, and preferably a phenylene group or a naphthalenylene group. When Q¹ represents a substituted divalent hydrocarbon group, examples of the substituent include a halogen atom (such as a fluorine atom).

Examples of the monovalent hydrocarbon group having 1 to 20 carbon atoms represented by R¹² include a monovalent chain hydrocarbon group having 1 to 10 carbon atoms, a monovalent alicyclic hydrocarbon group having 3 to 20 carbon atoms, and a monovalent aromatic hydrocarbon group having 6 to 20 carbon atoms. When R¹² represents a substituted monovalent hydrocarbon group, examples of the substituent include a halogen atom (such as a fluorine atom) and an alkoxy group.

Examples of the monovalent chain hydrocarbon group having 1 to 10 carbon atoms represented by R¹² to R¹⁴ include a linear or branched saturated hydrocarbon group having 1 to 10 carbon atoms and a linear or branched unsaturated hydrocarbon group having 1 to 10 carbon atoms. Among these, the monovalent chain hydrocarbon group having 1 to 10 carbon atoms represented by R¹² to R¹⁴ is preferably the linear or branched saturated hydrocarbon group having 1 to 10 carbon atoms.

Examples of the monovalent alicyclic hydrocarbon group having 3 to 20 carbon atoms represented by R¹² to R¹⁴ include a group in which one hydrogen atom is removed from a monocyclic saturated alicyclic hydrocarbon or unsaturated alicyclic hydrocarbon or an alicyclic polycyclic hydrocarbon having 3 to 20 carbon atoms. Specific examples of these alicyclic hydrocarbons include: monocyclic saturated alicyclic hydrocarbons such as cyclobutane, cyclopentane, cyclohexane, cycloheptane, and cyclooctane; monocyclic unsaturated alicyclic hydrocarbons such as cyclopentene, cyclohexene, cycloheptene, cyclooctene, and cyclodecene; and polycyclic alicyclic hydrocarbons such as bicyclo[2.2.1]heptane (norbornane), bicyclo[2.2.2]octane, tricyclo[3.3.1.1^{3,7}]decane (adamantane), and tetracyclo[6.2.1.1³⁶.0^{2,7}]dodecane.

Examples of the monovalent aromatic hydrocarbon group having 6 to 20 carbon atoms represented by R¹² include a group in which one hydrogen atom is removed from an aromatic ring such as benzene, naphthalene, anthracene, indene, and fluorene.

From the viewpoint of sufficiently removing a development residue and the viewpoint of increasing a difference in dissolution contrast between an exposed portion and an unexposed portion in a developer liquid, R¹² specifically preferably represents a monovalent substituted or unsubstituted hydrocarbon group having 1 to 8 carbon atoms, and more preferably a linear or branched monovalent saturated hydrocarbon group having 1 to 8 carbon atoms or a monovalent alicyclic hydrocarbon group having 3 to 8 carbon atoms.

Examples of the divalent alicyclic hydrocarbon group having 3 to 20 carbon atoms constituted by combining R¹³ and R¹⁴ together with a carbon atoms to which R¹³ and R¹⁴ are bonded include a group in which two hydrogen atoms are removed from one and the same carbon atom constituting a carbon ring of a monocyclic or polycyclic aliphatic hydrocarbon having the above number of carbon atoms. The divalent alicyclic hydrocarbon group constituted by combining R¹³ and R¹⁴ may be a monocyclic hydrocarbon group or a polycyclic hydrocarbon group. When the divalent alicyclic hydrocarbon group constituted by combining R¹³ and R¹⁴ is a polycyclic hydrocarbon group, this polycyclic hydrocarbon group may be a bridged alicyclic hydrocarbon group or a condensed alicyclic hydrocarbon group. The polycyclic hydrocarbon group may be a saturated hydrocarbon group or an unsaturated hydrocarbon group. The polycyclic hydrocarbon group is preferably a saturated hydrocarbon group.

Here, the term “bridged alicyclic hydrocarbon” refers to a polycyclic alicyclic hydrocarbon in which two carbon atoms not adjacent to each other among carbon atoms constituting an aliphatic ring are bonded with a bonding linkage having one or more carbon atoms. The term “condensed alicyclic hydrocarbon” refers to a polycyclic alicyclic hydrocarbon constituted with a plurality of aliphatic rings in a form of sharing a side (bond between two adjacent carbon atoms). Specific examples of the bridged alicyclic hydrocarbon include bicyclo[2.2.1]heptane (norbornane), bicyclo[2.2.2]octane, tricyclo[3.3.1.1^{3,7}]decane (adamantane), and tetracyclo[6.2.1.1^{3,6}.0^{2,7}]dodecane. Specific examples of the condensed alicyclic hydrocarbon include decahydronaphthalene and octahydronaphthalene.

Among the monocyclic alicyclic hydrocarbon groups, the saturated hydrocarbon group is preferably a cyclopentanediyl group, a cyclohexanediyl group, a cycloheptanediyl group, or a cyclooctanediyl group. The unsaturated hydrocarbon group is preferably a cyclopentenediyl group, a cyclohexenediyl group, a cycloheptenediyl group, or a cyclooctenediyl group. The polycyclic alicyclic hydrocarbon group is preferably a bridged aliphatic saturated hydrocarbon group, and more preferably a bicyclo[2.2.1]heptane-2,2-diyl group (norbornane-2,2-diyl group), a bicyclo[2.2.2]octane-2,2-diyl group, a tetracyclo[6.2.1.1^{3,6}.0^{2,7}]dodecanediyl group, or a tricyclo[3.3.1.1.^{3,7}]decane-2,2-diyl group (adamantane-2,2-diyl group).

In terms of ability to increase a difference in a dissolution rate between the exposed portion and the unexposed portion in the developer liquid and in terms of ability to form a finer pattern, the polymer (A) preferably has a structural unit represented by the following formula (3).

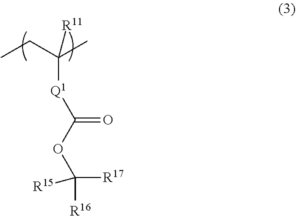

In the formula (3), R¹¹ represents a hydrogen atom, a fluorine atom, a methyl group, a trifluoromethyl group, or an alkoxyalkyl group. Q¹ represents a single bond or a substituted or unsubstituted divalent hydrocarbon group. R¹⁵ represents a substituted or unsubstituted monovalent hydrocarbon group having 1 to 8 carbon atoms. R¹⁶ and R¹⁷ each independently represent a monovalent chain hydrocarbon group having 1 to 8 carbon atoms or a monovalent alicyclic hydrocarbon group having 3 to 12 carbon atoms, or a divalent alicyclic hydrocarbon group having 3 to 12 carbon atoms constituted by combining R¹⁶ and R¹⁷ together with the carbon atom to which R¹⁶ and R¹⁷ are bonded.

In the formula (3), R¹¹ preferably represents a hydrogen atom or a methyl group, and more preferably a methyl group from the viewpoint of copolymerization properties of a monomer providing the structural unit represented by the formula (3). Specific examples and preferable examples of Q¹ include groups same as the group exemplified as Q¹ in the formula (2).

As specific examples of R¹⁵, R¹⁶, and R¹⁷, the examples having a corresponding number of carbon atoms in the description of R¹², R¹³, and R¹⁴ in the formula (2) are usable as a reference. Among these, R¹⁵ preferably represents a linear or branched monovalent saturated chain hydrocarbon group having 1 to 5 carbon atoms or a monovalent alicyclic hydrocarbon group having 3 to 8 carbon atoms, and more preferably a linear or branched monovalent saturated chain hydrocarbon group having 1 to 3 carbon atoms or a monovalent monocyclic aliphatic hydrocarbon group having 3 to 5 carbon atoms. R¹⁶ and R¹⁷ preferably represent a linear or branched monovalent chain saturated hydrocarbon group having 1 to 4 carbon atoms, a monovalent alicyclic hydrocarbon group having 3 to 12 carbon atoms, or a divalent alicyclic hydrocarbon group having 3 to 12 carbon atoms constituted by combining R¹⁶ and R¹⁷ together with the carbon atom to which R¹⁶ and R¹⁷ are bonded.

Among the above, in the structural unit represented by the formula (3), it is preferable that R¹⁵ and R¹⁶ represent an alkyl group having 1 to 4 carbon atoms and R¹⁷ represent a cycloalkyl group having 3 to 8 carbon atoms, a norbornyl group, or an adamantyl group, or it is preferable that R¹⁵ represent an alkyl group having 1 to 4 carbon atoms and R¹⁶ and R¹⁷ represent a cycloalkanediyl group having 3 to 8 carbon atoms, a norbornanediyl group, or an adamantanediyl group constituted by combining R¹⁶ and R¹⁷ together with the carbon atom to which R¹⁶ and R¹⁷ are bonded.

Specific examples of the structural unit (I) include structural units represented by each of the following formulae (2-1) to (2-7).

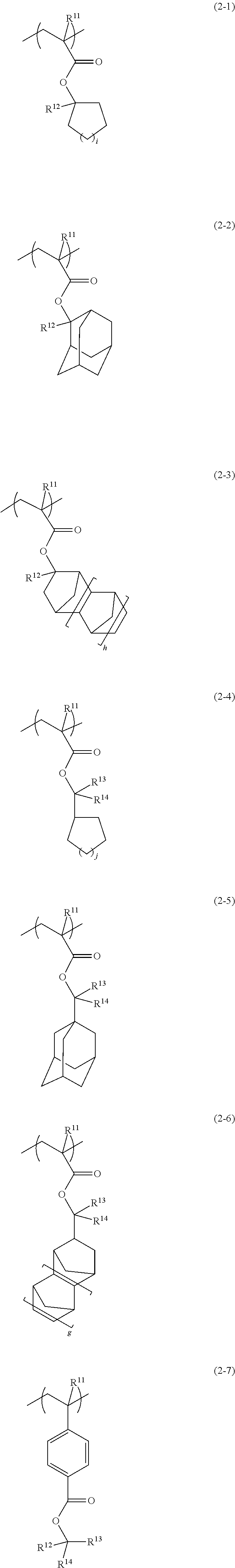

In the formulae (2-1) to (2-7), R¹¹ to R¹⁴ are as defined in the formula (2). “i” and “j” each independently represent an integer of 0 to 4. “h” and “g” each independently represent 0 or 1.

In the formulae (2-1) to (2-7), “i” and “j” preferably represent 1 or 2, and more preferably 1. “h” and “g” preferably represent 1. R¹² preferably represents a methyl group, an ethyl group, or an isopropyl group. R¹³ and R¹⁴ preferably represent a methyl group or an ethyl group.

A content proportion of the structural unit (I) is preferably 10 mol % or more, more preferably 25 mol % or more, and further preferably 35 mol % or more relative to all the structural units constituting the polymer (A). The content proportion of the structural unit (I) is preferably 80 mol % or less, more preferably 70 mol % or less, and further preferably 65 mol % or less relative to all the structural units constituting the polymer (A). Setting the content proportion of the structural unit (I) to be within the above range can more improve LWR performance, critical dimension uniformity (CDU) performance, which is an index of uniformity of a line width and a hole diameter, and pattern profile of the present composition.

When the polymer (A) has the structural unit represented by the formula (3) as the structural unit (I), a content proportion of the structural unit represented by the formula (3) is preferably 10 mol % or more, more preferably 30 mol % or more, and further preferably 50 mol % or more relative to all the structural units constituting the polymer (A). Setting the content proportion of the structural unit represented by the formula (3) to be within the above range can increase a difference in dissolution rate between the exposed portion and the unexposed portion in the developer liquid, and can form a finer pattern. Note that the polymer (A) may have only one type of the structural unit (I), or may have two or more types thereof in combination.

### [Other Structural Units]

The polymer (A) may further have a structural unit different from the structural unit (I) (hereinafter, also referred to as “other structural unit”) together with the structural unit (I). Examples of the other structural unit include the following structural unit (II) and structural unit (III).

### —Structural Unit (II)

The polymer (A) may further have a structural unit having a polar group (hereinafter, also referred to as “structural unit (II)”). The polymer (A) having the structural unit (II) can further easily regulate solubility of the polymer (A) in the developer liquid to improve lithography performance such as resolution. Examples of the structural unit (II) include a structural unit having at least one selected from the group consisting of a lactone structure, a cyclic carbonate structure, and a sultone structure (hereinafter, also referred to as “structural unit (II-1)” and a structural unit having a monovalent polar group (hereinafter, also referred to as “structural unit (II-2)”).

### —Structural Unit (II-1)

Introduction of the structural unit (II-1) into the polymer (A) can regulate solubility of the polymer (A) in the developer liquid, improve adhesiveness to a resist film, and further improve etching resistance. Examples of the structural unit (II-1) include structural units represented by the following formulae (4-1) to (4-10).

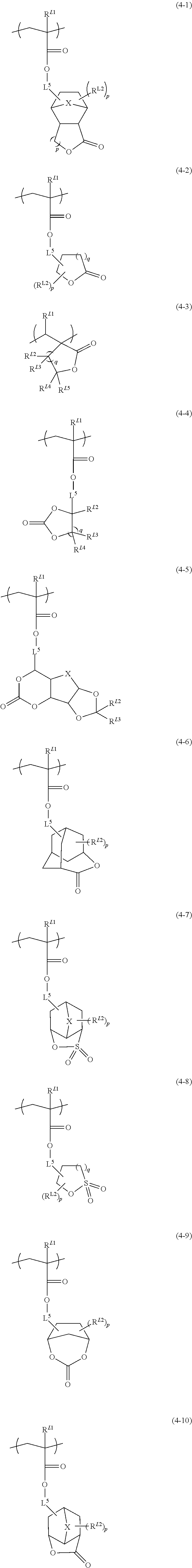

In the formulae (4-1) to (4-10), R^{L1} represents a hydrogen atom, a fluorine atom, a methyl group, a trifluoromethyl group, or an alkoxyalkyl group. R^{L2} and R^{L3} each independently represent a hydrogen atom, an alkyl group having 1 to 4 carbon atoms, a cyano group, a trifluoromethyl group, a methoxy group, a methoxycarbonyl group, a hydroxy group, a hydroxymethyl group, or a dimethylamino group. R^{L4} and R^{L5} each independently represent a hydrogen atom, an alkyl group having 1 to 4 carbon atoms, a cyano group, a trifluoromethyl group, a methoxy group, a methoxycarbonyl group, a hydroxy group, a hydroxymethyl group, or a dimethylamino group, or a divalent alicyclic hydrocarbon group having 3 to 8 carbon atoms constituted by combining R^{L4} and R^{L5} together with the carbon atom to which R^{L4} and R^{L5} are bonded. L⁵ represents a single bond or a divalent linking group. X represents an oxygen atom or a methylene group. “p” represents an integer of 0 to 3. “q” represents an integer of 1 to 3.

Examples of the divalent alicyclic hydrocarbon group having 3 to 8 carbon atoms constituted by combining R^{L4} and R^{L5} together with the carbon atom to which R^{L4} and R^{L5} are bonded include groups having 3 to 8 carbon atoms in the description of R¹³ and R¹⁴ in the formula (2). One or more hydrogen atoms on this alicyclic hydrocarbon group may be replaced by a hydroxy group.

Examples of the divalent linking group represented by L⁵ include a linear or branched divalent chain hydrocarbon group having 1 to 10 carbon atoms, a divalent alicyclic hydrocarbon group having 4 to 12 carbon atoms, or a group constituted with one or more of these hydrocarbon groups and at least one group of —CO—, —O—, —NH—, and —S—.

The structural unit (II-1) is preferably the structural unit represented by the formula (4-2), the formula (4-4), the formula (4-6), the formula (4-7), or the formula (4-10) among the formulae (4-1) to (4-10).

When the polymer (A) has the structural unit (II-1), a content proportion of the structural unit (II-1) is preferably 80 mol % or less, more preferably 70 mol % or less, and further preferably 65 mol % or less relative to all the structural units constituting the polymer (A). When the polymer (A) has the structural unit (II-1), the content proportion of the structural unit (II-1) is preferably 2 mol % or more, more preferably 5 mol % or more, and further preferably 10 mol % or more relative to all the structural units constituting the polymer (A). Setting the content proportion of the structural unit (II-1) to be within the above range can more improve lithography performance of the present composition such as resolution.

—Structural Unit (II-2)

The structural unit (II-2) may be introduced into the polymer (A) for regulating solubility of the polymer (A) in the developer liquid to improve lithography performance of the present composition such as resolution. Examples of the polar group in the structural unit (II-2) include a hydroxy group, a carboxy group, a cyano group, a nitro group, and a sulfonamide group. Among these, a hydroxy group and a carboxy group are preferable, and a hydroxy group (particularly, an alcoholic hydroxy group) is more preferable. Note that the structural unit (II-2) is a structural unit different from a structural unit having a phenolic hydroxy group (structural unit (III)) described below.

Here, the term “phenolic hydroxy group” herein refers to a group in which a hydroxy group is directly bonded to an aromatic hydrocarbon structure. The term “alcoholic hydroxy group” refers to a group in which a hydroxy group is directly bonded to an aliphatic hydrocarbon structure. In the alcoholic hydroxy group, the aliphatic hydrocarbon structure to which the hydroxy group is bonded may be a chain hydrocarbon group or an alicyclic hydrocarbon group.

Examples of the structural unit (II-2) include structural units represented by the following formula. The structural unit (II-2) is not limited thereto.

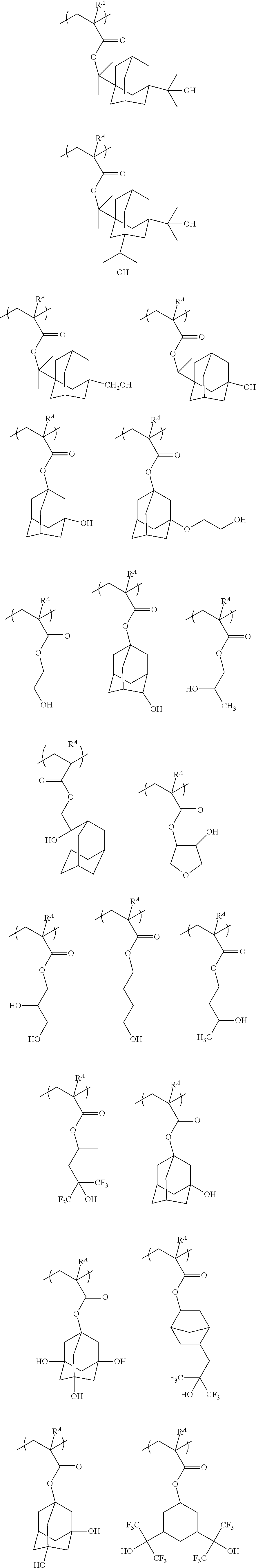

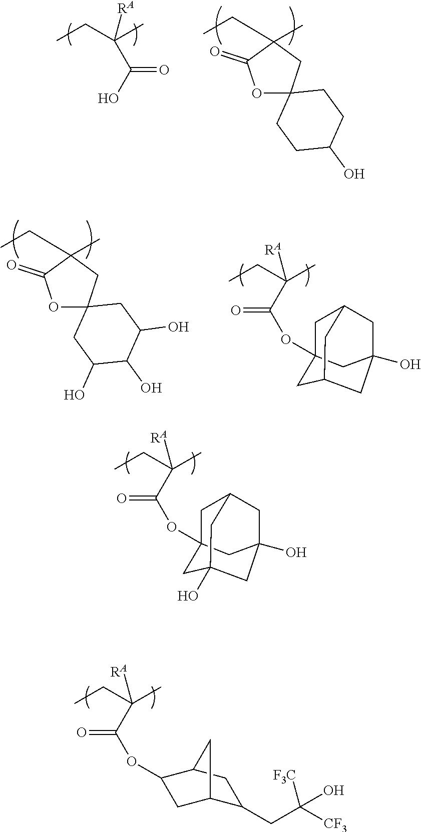

In the formula, R^{A} represents a hydrogen atom, a fluorine atom, a methyl group, a trifluoromethyl group, or an alkoxyalkyl group.

When the polymer (A) has the structural unit (II-2), a content proportion of the structural unit (II-2) is preferably 2 mol % or more, and more preferably 5 mol % or more relative to all the structural units constituting the polymer (A). The content proportion of the structural unit (II-2) is preferably 30 mol % or less, and more preferably 25 mol % or less relative to all the structural units constituting the polymer (A). Setting the content proportion of the structural unit (II-2) to be within the above range can further improve lithography performance of the present composition such as resolution.

—Structural Unit (III)

The polymer (A) may further have a structural unit having a phenolic hydroxy group (hereinafter, also referred to as “structural unit (III)”). The polymer (A) having the structural unit (III) is preferable in terms of improvement of etching resistance and improvement of a difference of developer liquid solubility (dissolution contrast) between the exposed portion and the unexposed portion.

Particularly, in pattern formation using exposure with radiation having a wavelength of 50 nm or shorter, such as electron beam and EUV, the polymer (A) having the structural unit (III) can be preferably used. When applied for the pattern formation using exposure with radiation having a wavelength of 50 nm or shorter, the polymer (A) preferably has the structural unit (III).

The structural unit (III) is not particularly limited as long as the unit has a phenolic hydroxy group. Specific examples of the structural unit (III) include a structural unit derived from hydroxystyrene or a derivative thereof and a structural unit derived from a (meth)acryl compound having a hydroxybenzene structure.

When the polymer having the structural unit (III) is obtained as the polymer (A), it is acceptable that polymerization is performed in a state where the phenolic hydroxy group is protected with an alkali-dissociable group etc., and then hydrolysis is performed for deprotection to allow the polymer (A) to have the structural unit (III). The structural unit to yield the structural unit (III) by hydrolysis is preferably at least one selected from the group consisting of a structural unit represented by the following formula (5-1) and a structural unit represented by the following formula (5-2).

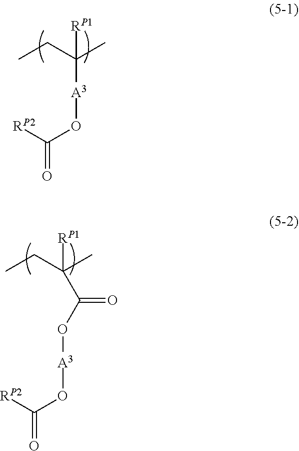

In the formulae (5-1) and (5-2), R^{P1} represents a hydrogen atom, a fluorine atom, a methyl group, a trifluoromethyl group, or an alkoxyalkyl group. A³ represents a substituted or unsubstituted divalent aromatic ring group. R^{P2} represents a monovalent hydrocarbon group having 1 to 20 carbon atoms or an alkoxy group.

The aromatic ring group represented by A³ is a group in which two hydrogen atoms are removed from a ring moiety of a substituted or unsubstituted aromatic ring. This aromatic ring is preferably a hydrocarbon ring, and examples thereof include aromatic hydrocarbon rings such as benzene, naphthalene, and anthracene. Among these, A³ preferably represents a group in which two hydrogen atoms are removed from a ring moiety of a substituted or unsubstituted benzene or naphthalene, and more preferably a substituted or unsubstituted phenylene group. Examples of the substituent include a halogen atom such as a fluorine atom.

Examples of the monovalent hydrocarbon group having 1 to 20 carbon atoms represented by R^{P2} include the groups exemplified as the monovalent hydrocarbon group having 1 to 20 carbon atoms of R¹² in the structural unit (I). Examples of the alkoxy group include a methoxy group, an ethoxy group, and a tert-butoxy group. Among these, R^{P2} preferably represents an alkyl group or an alkoxy group, and specifically preferably a methyl group or a tert-butoxy group.

When a radiation-sensitive composition for exposure with radiation having a wavelength of 50 nm or shorter is obtained, a content proportion of the structural unit (III) in the polymer (A) is preferably 15 mol % or more, and more preferably 20 mol % or more relative to all the structural units constituting the polymer (A). The content proportion of the structural unit (III) in the polymer (A) is preferably 65 mol % or less, and more preferably 60 mol % or less relative to all the structural units constituting the polymer (A).

### —Synthesis of Polymer (A)

The polymer (A) can be synthesized by, for example, polymerizing monomers providing the structural units by using a radical polymerization initiator etc. in an appropriate solvent.

Examples of the radical polymerization initiator include: azo-type radical initiators such as azobisisobutyronitrile (AIBN), 2,2′-azobis(4-methoxy-2,4-dimethyl valeronitrile), 2,2′-azobis(2-cyclopropylpropionitrile), 2,2′-azobis(2,4-dimethylvaleronitrile), and dimethyl 2,2′-azobisisobutyrate; and peroxide-type radical initiators such as benzoyl peroxide, t-butyl hydroperoxide, and cumene hydroperoxide. Among these, AIBN and dimethyl 2,2′-azobisisobutyrate are preferable, and AIBN is more preferable. These radical initiators may be used singly, or two or more thereof may be mixed for use.

Examples of the solvent used for the polymerization include alkanes, cycloalkanes, aromatic hydrocarbons, halogenated hydrocarbons, saturated carboxylic acid esters, ketones, ethers, and alcohols. Specific examples thereof include: alkanes such as n-pentane, n-hexane, n-heptane, n-octane, n-nonane, and n-decane; cycloalkanes such as cyclohexane, cycloheptane, cyclooctane, decalin, and norbornane; aromatic hydrocarbons such as benzene, toluene, xylene, ethylbenzene, and cumene; halogenated hydrocarbons such as chlorobutanes, bromohexanes, dichloroethanes, hexamethylene dibromide, and chlorobenzene; saturated carboxylic acid esters such as ethyl acetate, n-butyl acetate, i-butyl acetate, and methyl propionate; ketones such as acetone, methyl ethyl ketone, 4-methyl-2-pentanone, and 2-heptanone; ethers such as tetrahydrofuran, dimethoxyethanes, and diethoxyethanes; and alcohols such as methanol, ethanol, 1-propanol, 2-propanol, and 4-methyl-2-pentanol. The solvent used for the polymerization may be used singly or in combination of two or more thereof.

The reaction temperature in the polymerization is typically 40° C. to 150° C., and preferably 50° C. to 120° C. The reaction time is typically 1 hour to 48 hours, and preferably 1 hour to 24 hours.

A weight-average molecular weight (Mw) of the polymer (A) in terms of polystyrene by gel permeation chromatography (GPC) is preferably 1,000 or more, more preferably 2,000 or more, further preferably 3,000 or more, and furthermore preferably 4,000 or more. Mw of the polymer (A) is preferably 50,000 or less, more preferably 30,000 or less, further preferably 20,000 or less, and furthermore preferably 15,000 or less. Setting Mw of the polymer (A) to be within the above range is preferable in terms of ability to improve coatability of the present composition, in terms of ability to improve heat resistance of the resist film to be obtained, and in terms of ability to sufficiently inhibit development defects.

A ratio (Mw/Mn) of the polymer (A) of Mw to a number-average molecular weight (Mn) in terms of polystyrene by GPC is preferably 5.0 or less, more preferably 3.0 or less, and further preferably 2.0 or less. Mw/Mn is typically 1.0 or more.

A content proportion of the polymer (A) in the present composition is preferably 70 mass % or more, more preferably 75 mass % or more, and further preferably 80 mass % or more relative to the total amount of solid contents contained in the present composition (namely a total mass of components contained in the present composition except for a solvent content). The content proportion of the polymer (A) is preferably 99 mass % or less, more preferably 98 mass % or less, and further preferably 95 mass % or less relative to the total amount of the solid contents contained in the present composition. The polymer (A) preferably constitutes a base resin of the present composition. The term “base resin” herein refers to a polymer component accounting for 50 mass % or more in the total amount of the solid contents contained in the present composition. The present composition may contain only one type of the polymer (A) or may contain two or more types of the polymer (A).

### <Compound (B)>

The compound (B) is a compound represented by the following formula (1).

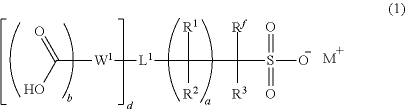

In the formula (1), L¹ represents a group having a (thio)acetal ring formed by replacing each of two methylene groups of a monocyclic saturated aliphatic hydrocarbon ring by a (thio)ether bond so that two oxygens, two sulfurs, or one oxygen and one sulfur are bonded to one and the same carbon, or represents a group represented by the following formula (L-2):

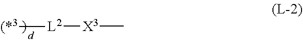

wherein, in the formula (L-2), L² represents a bridged alicyclic group having 7 or more carbon atoms. X³ represents a single bond, an oxygen atom, a sulfur atom, or —SO₂—. “d” represents 1 or 2. “*³” represents a chemical bond to W¹ or the carboxy group. W¹ represents a single bond or a (b+1)-valent organic group having 1 to 40 carbon atoms. R¹, R², and R³ each independently represent a hydrogen atom, a hydrocarbon group having 1 to 10 carbon atoms, a fluorine atom, or a fluoroalkyl group. R^{f} represents a fluorine atom or a fluoroalkyl group. “a” represents an integer of 0 to 8. “b” represents an integer of 1 to 4. “d” represents 1 or 2. When L¹ represents the group represented by the formula (L-2), “d” in the formula (1) and “d” in the formula (L-2) are the same values. When “a” represents 2 or more, a plurality of R¹ are the same or different, and a plurality of R² are the same or different. When “d” represents 2, a plurality of W¹ are the same or different, and a plurality of “b” are the same or different. M⁺ represents a monovalent cation.

The compound (B) can function as a radiation-sensitive acid generator. The radiation-sensitive acid generator (hereinafter, also simply referred to as “acid generator”) is a substance to generate an acid in a composition by irradiating the radiation-sensitive composition with radiation. The acid generator is typically an onium salt composed of a radiation-sensitive onium cation and an organic anion, and preferably a compound that generates a strong acid such as a sulfonic acid, an imide acid, and a methide acid to induce dissociation of the acid-releasable group under a normal condition. Note that “normal condition” herein refers to a condition of performing post exposure baking (PEB) at 110° C. for 60 seconds. It is preferable that the compound (B) be blended in the present composition together with the polymer (A) and the acid-releasable group in the polymer (A) be eliminated by the acid generated from the compound (B) to generate the acid group to make a difference in a dissolution rate of the polymer (A) in the developer liquid between the exposed portion and the unexposed portion.

The present composition containing the compound (B) as the acid generator can appropriately shorten a diffusion length of the acid generated by exposing the present composition. According to this, the present composition can form the resist film having excellent lithography performance such as LWR performance and CDU performance and pattern rectangularity while exhibiting high sensitivity. In addition, an insoluble component remained in the pattern after the development can be reduced to consequently reduce development defects.

In the formula (1), the group represented by L¹ is a group having a (thio)acetal ring or represented by the formula (L-2). Here, the (thio)acetal ring refers to a cyclic structure containing a ring formed by replacing each of two methylene groups constituting a monocyclic saturated aliphatic hydrocarbon ring (for example, a cyclopentane ring, a cyclohexane ring, a cycloheptane ring, a cyclooctane ring, etc.) by a (thio)ether bond so that two oxygens, two sulfurs, or one oxygen and one sulfur are bonded to one and the same carbon (hereinafter, also referred to as “(thio)acetal ring”). Note that the (thio)acetal ring excludes: a ring having a heteroatom other than oxygen and sulfur in the cyclic skeleton; and a ring having a carbon to which a heteroatom is directly bonded (for example, a carbon to which an oxo group is bonded) in the cyclic skeleton. Therefore, for example, a ring having an ester bond (—C(═O)—O—) in the cyclic skeleton does not correspond to the “(thio)acetal ring”.

A number of ring member of the (thio)acetal ring is preferably 5 to 18, more preferably 5 to 10, and further preferably 5 or 6. The cyclic (thio)acetal structure may have a structure in which the carboxy group in the formula (1) is directly bonded to the cyclic moiety (namely the (thio)acetal ring), or may have a structure in which a substituent other than the carboxy group is bonded to the (thio)acetal ring. Examples of the other substituent include a substituted or unsubstituted monovalent hydrocarbon group having 1 to 10 carbon atoms, and among these, a monovalent chain hydrocarbon group having 1 to 10 carbon atoms is preferable, and an alkyl group having 1 to 3 carbon atoms is more preferable.

When L¹ represents the group having the (thio)acetal ring, L¹ may be any as long as L¹ has the cyclic (thio)acetal structure. Therefore, L¹ may be, for example, a group having a divalent linking group together with the (thio)acetal ring and is bonded to the group “—C(R¹) (R²)—” or the group “—C(R^{f}) (R³)—” via the divalent linking group. The (thio)acetal ring in L¹ may be a single ring or may be a part of rings constituting a polycyclic structure. When the (thio)acetal ring in L¹ is a part of rings constituting a polycyclic structure, the (thio)acetal ring in L¹ may be a part of rings constituting a condensed cyclic structure condensed with another ring or may be a part of rings constituting a spiro-ring structure sharing a carbon with another ring. When the (thio)acetal ring in L¹ is a part of rings constituting a condensed cyclic structure condensed with another ring, the other ring may be a monocyclic aliphatic ring or aromatic ring, or may be a bridged aliphatic ring. When the (thio)acetal ring in L¹ is a part of rings constituting a spiro-ring structure sharing a carbon with another ring, the other ring may be a monocyclic aliphatic ring or aromatic ring, or may be a bridged aliphatic ring. A ring forming a polycyclic structure together with the (thio)acetal ring may have a substituent. Examples of the substituent include a halogen atom (for example, a fluorine atom, a chlorine atom, a bromine atom, an iodine atom, etc.), a hydroxy group, a carboxy group, a cyano group, an alkoxy group, an alkoxycarbonyl group, an alkylcarbonyloxy group, a cycloalkoxycarbonyl group, and a cycloalkylcarbonyloxy group.

The (thio)acetal ring in L¹ is preferably an acetal ring in which two methylene groups constituting a monocyclic saturated aliphatic hydrocarbon ring are both replaced by an ether bond from the viewpoint of ease of synthesis.

When L¹ is the group represented by the formula (L-2), the bridged alicyclic group having 7 or more carbon atoms represented by L² may be an alicyclic hydrocarbon group or an aliphatic heterocyclic group. Here “bridged alicyclic group” refers to an n-valent group (“n” represents an integer of 1 or more) in which “n” hydrogen atoms are removed from a polycyclic alicyclic hydrocarbon or an aliphatic heteroring in which two carbon atoms not adjacent to each other among carbon atoms constituting the alicyclic hydrocarbon or the aliphatic heteroring are linked with a linking chain having one or more atoms. The bridged alicyclic group may have a substituent in the cyclic moiety. A number of carbon atoms of the ring (bridged alicyclic hydrocarbon or aliphatic heteroring) in the bridged alicyclic group is preferably 7 or more, and more preferably 8 or more. The number of carbon atoms of the ring in the bridged alicyclic group is 20 or less, for example.

Specific examples of the ring in the bridged alicyclic group represented by L² include: bridged alicyclic hydrocarbons such as bicyclo[2.2.1]heptane, bicyclo[2.2.2]octane, tricyclo[3.3.1.1³⁷]decane, and tetracyclo[6.2.1.1^{3,6}.0^{2,7}]dodecane; and bridged aliphatic heterorings such as 7-oxabicyclo[2.2.1]heptane, 7-azabicyclo[2.2.1]heptane, and 9-oxatetracyclo[6.2.1.1^{3,6}.0.^{2,7}]dodecane. When the bridged alicyclic group represented by L² has a substituent in the cyclic moiety, examples of the substituent include groups same as the groups exemplified as the substituent that the ring forming a polycyclic structure together with the (thio)acetal ring may have.

X³ represents a single bond, an oxygen atom, a sulfur atom, or —SO₂—. Among these, X³ preferably represents a single bond or an oxygen atom from the viewpoint of ease of synthesis.

The (b+1)-valent organic group having 1 to 40 carbon atoms represented by W¹ may be a group composed of only a chain structure, or may be a group having a cyclic structure. Examples of the (b+1)-valent organic group include: a substituted or unsubstituted (b+1)-valent hydrocarbon group having 1 to 40 carbon atoms, a (b+1)-valent group in which any methylene group in a substituted or unsubstituted hydrocarbon group is replaced by —O—, —CO—, or —COO—; a (b+1)-valent group having an aliphatic heterocyclic structure having 3 to 40 carbon atoms (excluding the cyclic (thio)acetal structure); and a (b+1)-valent group having an aromatic heterocyclic structure having 4 to 40 carbon atoms.

When W¹ represents the (b+1)-valent hydrocarbon group, examples of the hydrocarbon group include a (b+1)-valent chain hydrocarbon group having 1 to 40 carbon atoms, a (b+1)-valent alicyclic hydrocarbon group having 3 to 40 carbon atoms, and a (b+1)-valent aromatic hydrocarbon group having 6 to 40 carbon atoms. Specific examples thereof include a group in which “b” hydrogen atoms are further removed from the monovalent hydrocarbon group exemplified in the description of R¹² in the formula (2). Among these, the (b+1)-valent hydrocarbon group represented by W¹ is preferably a (b+1)-valent chain hydrocarbon group having 1 to 6 carbon atoms, a (b+1)-valent alicyclic hydrocarbon group having 3 to 20 carbon atoms, or a (b+1)-valent aromatic hydrocarbon group having 6 to 20 carbon atoms. Among these, from the viewpoint of inhibiting diffusion of the acid generated by exposure and the viewpoint of inhibiting occurrence of development defects, the (b+1)-valent hydrocarbon group represented by W¹ is more preferably a (b+1)-valent alicyclic hydrocarbon group having 3 to 20 carbon atoms or a (b+1)-valent aromatic hydrocarbon group having 6 to 20 carbon atoms, further preferably a (b+1)-valent polycyclic alicyclic hydrocarbon group having 7 to 20 carbon atoms or a (b+1)-valent aromatic hydrocarbon group having 6 to 20 carbon atoms, and furthermore preferably a (b+1)-valent aromatic hydrocarbon group having 6 to 20 carbon atoms.

When W¹ represents the substituted (b+1)-valent hydrocarbon group, examples of the substituent include a halogen atom (for example, a fluorine atom, a chlorine atom, a bromine atom, an iodine atom, etc.), a hydroxy group, a cyano group, an alkoxy group, and an alkoxycarbonyl group.

When W¹ represents the (b+1)-valent group having an aliphatic heterocyclic structure, examples of the aliphatic heterocyclic structure in W¹ include a cyclic ether structure (excluding the cyclic (thio)acetal structure), a lactone structure, a cyclic carbonate structure, a sultone structure, and a thioxane structure. The aliphatic heterocyclic structure may be any of a monocyclic structure and a polycyclic structure, and may be any of a bridged structure, a condensed cyclic structure, and a spiro-ring structure. The aliphatic heterocyclic structure represented by W¹ may be a combination of two or more of a bridged structure, a condensed cyclic structure, and a spiro-ring structure.

From the viewpoint of inhibiting diffusion of the acid derived from the compound (B) to improve quality of the resist pattern and from the viewpoint of increasing hydrophobicity of the film while improving transparency of the resist film to more increase a difference in dissolution rate in the developer liquid between the exposed portion and the unexposed portion, the (b+1)-valent organic group represented by W¹ is preferably a (b+1)-valent group having a cyclic structure. Specifically, the (b+1)-valent organic group represented by W¹ preferably has an alicyclic hydrocarbon structure, an aliphatic heterocyclic structure, an aromatic hydrocarbon structure, or an aromatic heterocyclic structure, and more preferably has an alicyclic hydrocarbon structure, an aliphatic heterocyclic structure, or an aromatic hydrocarbon structure.

Specific examples of a case where the (b+1)-valent organic group represented by W¹ is a group having an alicyclic hydrocarbon structure include a group having a cyclobutane structure, a cyclopentane structure, a cyclohexane structure, a cycloheptane structure, a cyclooctane structure, a cyclopentene structure, a cyclohexene structure, a bicyclo[2.2.1]heptane structure, a bicyclo[2.2.2]octane structure, a tricyclo[3.3.1.1^{3,7}]decane structure, a tetracyclo[6.2.1.1^{3,6}.0^{2,7}]dodecane structure, a decahydronaphthalene structure, or an octahydronaphthalene structure.

Specific examples of the case where the (b+1)-valent organic group represented by W¹ is a group having an aliphatic heterocyclic structure include a group having a lactone structure, a cyclic carbonate structure, a sultone structure or a thioxane structure.

Specific examples of the case where the (b+1)-valent organic group represented by W¹ is a group having an aromatic hydrocarbon structure include a group having a benzene ring structure, a naphthalene ring structure, an indene ring structure, an anthracene ring structure, a phenanthrene ring structure, or a fluorene ring structure.

Specific examples of the case where the (b+1)-valent organic group represented by W¹ is a group having an aromatic heterocyclic structure include a group having a furan structure or a thiophene structure.

Among the above, the (b+1)-valent organic group represented by W¹ more preferably has a bridged aliphatic saturated hydrocarbon structure, a bridged aliphatic heterocyclic structure, or an aromatic hydrocarbon structure, and further preferably has an aromatic hydrocarbon structure. W¹ preferably has no fluorine atom from the viewpoint of sensitivity.

In one or more partial structures “—W¹—(COOH)_{b}” bonded to L¹ in the formula (1), when W¹ represents a (b+1)-valent organic group having 1 to 40 carbon atoms, this W¹ is preferably a group having a cyclic structure, and one or a plurality of carboxy groups are preferably directly bonded to the ring in W¹. In this case, the ring in W¹ is preferably an alicyclic hydrocarbon ring, an aliphatic heteroring, or an aromatic hydrocarbon group, more preferably a bridged aliphatic saturated hydrocarbon ring, a bridged aliphatic heteroring, or an aromatic hydrocarbon ring, and further preferably an aromatic hydrocarbon ring. Specific examples of these rings are as described above.

In one or more partial structures “—W¹—(COOH)_{b}” bonded to L¹ in the formula (1), when W¹ represents a single bond and L¹ represents the group having the (thio)acetal ring, L¹ preferably has a ring (hereinafter, also referred to as “ring R^{X}”) forming a condensed cyclic structure or a spiro-ring structure together with the (thio)acetal ring in L¹, and the carboxy group is preferably bonded to the ring R^{X} or the (thio)acetal ring. The ring R^{X} is preferably an aliphatic hydrocarbon ring, an aromatic hydrocarbon ring, or an aliphatic heteroring, and these may have any of a monocyclic or polycyclic structure. When the ring R^{X} is polycyclic, the ring R^{X} may be a ring having any of a bridged structure, a condensed cyclic structure, and a spiro-ring structure. When the ring R^{X} is polycyclic, the ring R^{X} may be a combination of two or more of a bridged structure, a condensed cyclic structure, and a spiro-ring structure.

Specific examples of the ring R^{X} include: monocyclic aliphatic hydrocarbon rings such as cyclobutane, cyclopentane, cyclohexane, cycloheptane, cyclooctane, cyclopentene, cyclohexene, cycloheptene, cyclooctene, and cyclodecene; polycyclic aliphatic hydrocarbon rings such as bicyclo[2.2.1]heptane (norbornane), bicyclo[2.2.2]octane, tricyclo[3.3.1.1^{3,7}]decane (adamantane), tetracyclo[6.2.1.1^{3,6}.0^{2,7}]dodecane, decahydronaphthalene, and octahydronaphthalene; polycyclic saturated heterorings having a lactone structure, a cyclic carbonate structure, a sultone structure, or a thioxane structure; and polycyclic aromatic hydrocarbon rings such as a naphthalene ring, an indene ring, an anthracene ring, a phenanthrene ring, and a fluorene ring.

From the viewpoint of inhibiting diffusion of the acid generated by exposure, when W¹ in one or more partial structures “—W—(COOH)_{b}” bonded to L¹ in the formula (1) represents a single bond, the ring R^{X} in L¹ is preferably a polycyclic aliphatic hydrocarbon ring, a polycyclic saturated heteroring, or a polycyclic aromatic hydrocarbon ring, more preferably a bridged aliphatic saturated hydrocarbon ring, a bridged aliphatic heteroring, or a polycyclic aromatic hydrocarbon ring, and further preferably a bridged aliphatic saturated hydrocarbon ring. When W¹ in one or more partial structures “—W—(COOH)_{b}” bonded to L¹ in the formula (1) represents a single bond and a carboxy group is bonded to the ring in L¹, the carboxy group is preferably directly bonded to the ring R^{X} in terms of ability to more increase the effect of inhibiting development defects.

The ring R^{X} may have a substituent other than the carboxy group. Examples of the substituent include a halogen atom (for example, a fluorine atom, a chlorine atom, a bromine atom, an iodine atom, etc.), a hydroxy group, a cyano group, an alkoxy group, and an alkoxycarbonyl group.

When L¹ represents the group having the (thio)acetal ring in the formula (1), a direction of the (thio)acetal ring in L¹ is not particularly limited. Thus, the (thio)acetal ring in L¹ may be arranged so that the carbon to which two oxygens, two sulfurs, or one oxygen and one sulfur are bonded is positioned on a “—SO₃—” side, or may be arranged in the opposite side. From the viewpoint of ease of synthesis, the (thio)acetal ring in L¹ is preferably arranged so that the carbon to which two oxygens, two sulfurs, or one oxygen and one sulfur are bonded is positioned on a side opposite to “—SO₃—” (that is, the W¹ or carboxy group side in the formula (1)). Specifically, when L¹ represents the group having the (thio)acetal ring, L¹ preferably represents a group represented by the following formula (L-1). Note that, in the following formula (L-1), a carbon to which X¹ and X² are bonded is the “carbon to which two oxygens, two sulfurs, or one oxygen and one sulfur are bonded” in the (thio)acetal ring.

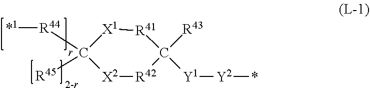

In the formula (L-1), X¹ and X² each independently represent an oxygen atom or a sulfur atom. R⁴¹ represents a single bond or an alkanediyl group having 1 to 10 carbon atoms. “r” represents 1 or 2. When “r” represents 1, R⁴⁴ represents a single bond and R⁴⁵ represents a hydrogen atom or a monovalent hydrocarbon group having 1 to 10 carbon atoms, or R⁴⁴ and R⁴⁵ represent a cyclic structure forming a spiro-ring structure by combining R⁴⁴ and R⁴⁵ together with the carbon atom to which R⁴⁴ and R⁴⁵ are bonded and the (thio) acetal ring in the formula (L-1). When “r” represents 2, R⁴⁴ represents a single bond. Among partial structures “—W¹—(COOH)_{b}” bonded to L¹ in the formula (1), “b” in a partial structure in which W¹ represents a single bond represents 1. R⁴², R⁴³, Y¹, and Y² satisfy the following (i), (ii), or (iii).
(i) R⁴² represents an alkanediyl group having 1 to 10 carbon atoms. R⁴³ represents a hydrogen atom or a monovalent hydrocarbon group having 1 to 10 carbon atoms. Y¹ represents a single bond or a divalent linking group. Y² represents a single bond.
(ii) R⁴² and Y¹ represent a cyclic structure forming a condensed cyclic structure by combining R⁴² and Y¹ together with the carbon atom to which R⁴² and Y¹ are bonded and the (thio)acetal ring in the formula (L-1). R⁴³ represents a hydrogen atom or a monovalent hydrocarbon group having 1 to 10 carbon atoms. Y² represents a single bond or a divalent linking group.
(iii) R⁴³ and Y¹ represent a cyclic structure forming a spiro-ring structure by combining R⁴³ and Y¹ together with the carbon atom to which R⁴³ and Y¹ are bonded and the (thio)acetal ring in the formula (L-1). R⁴² represents an alkanediyl group having 1 to 10 carbon atoms. Y² represents a single bond or a divalent linking group.

“*¹” represents a chemical bond to W¹ or the carboxy group in the formula (1). “*” represents a chemical bond.

In the formula (L-1), X¹ and X² preferably represent both an oxygen atom or both a sulfur atom, and more preferably both an oxygen atom.

The alkanediyl group having 1 to 10 carbon atoms represented by R⁴¹ may be linear or branched. From the viewpoint of ease of synthesis, this alkanediyl group preferably has 1 to 3 carbon atoms, and is more preferably a methylene group.

R⁴¹ preferably represents a single bond or a linear or branched alkanediyl group having 1 to 3 carbon atoms, and more preferably a single bond or a methylene group.

As for R⁴⁴ and R⁴⁵, when R⁴⁴ and R⁴⁵ represent a cyclic structure forming a spiro-ring structure by combining R⁴⁴ and R⁴⁵ together with the carbon atom to which R⁴⁴ and R⁴⁵ are bonded and the (thio)acetal ring, specific examples of the ring forming the spiro-ring structure together with the (thio)acetal ring include the rings exemplified in the description of the ring R^{X}. Among these, the spiro-ring structure constituted by combining R⁴⁴ and R⁴⁵ together with the (thio)acetal ring specifically preferably has a bridged aliphatic saturated hydrocarbon ring, a bridged aliphatic heteroring, or a polycyclic aromatic hydrocarbon ring, more preferably a bridged aliphatic saturated hydrocarbon ring or a bridged aliphatic heteroring, and further preferably a bridged aliphatic saturated hydrocarbon ring.

When R⁴⁵ represents the monovalent hydrocarbon group having 1 to 10 carbon atoms, specific examples of this monovalent hydrocarbon group include groups same as the examples having a corresponding number of carbon atoms among the monovalent hydrocarbon groups exemplified in the description of R¹² in the formula (2). Among these, the monovalent hydrocarbon group having 1 to 10 carbon atoms represented by R⁴⁵ is preferably a monovalent chain hydrocarbon group having 1 to 10 carbon atoms, a monovalent alicyclic hydrocarbon group having 3 to 10 carbon atoms, or a monovalent aromatic hydrocarbon group having 6 to 10 carbon atoms, more preferably a linear or branched saturated chain hydrocarbon group having 1 to 4 carbon atoms or a monocyclic aliphatic hydrocarbon group having 3 to 8 carbon atoms, and further preferably an alkyl group having 1 to 3 carbon atoms.

When R⁴², R⁴³, Y¹, and Y² satisfy the above (i), the alkanediyl group having 1 to 10 carbon atoms represented by R⁴² may be linear or branched. R⁴² preferably represents an alkanediyl group having 1 to 3 carbon atoms, and more preferably a methylene group from the viewpoint of ease of synthesis.

When the group represented by Y¹ is a divalent linking group, examples of the divalent linking group include a carbonyl group, a carbonyloxy group, *²—R²⁰—O—, *²—R²⁰—CO—, *²—R²⁰—CO—O—, and *²—R²—O—CO— (wherein R²⁰ represents an alkanediyl group having 1 to 3 carbon atoms, and “*²” represents a chemical bond to carbon to which R⁴² and R⁴³ are bonded). From the viewpoint of inhibiting diffusion of the acid generated by exposing the present composition and from the viewpoint of ease of synthesis, Y¹ preferably represents a single bond, a carbonyl group, a carbonyloxy group, or —CH₂—O—CO—, and more preferably a single bond.

Specific examples of the monovalent hydrocarbon group having 1 to 10 carbon atoms represented by R⁴³ include groups same as the examples having a corresponding number of carbon atoms among the monovalent hydrocarbon groups exemplified in the description of R¹² in the formula (2). Among these, R⁴³ preferably represents a hydrogen atom, a linear or branched saturated chain hydrocarbon group having 1 to 4 carbon atoms, or a monocyclic aliphatic hydrocarbon group having 3 to 8 carbon atoms.

When R⁴², R⁴³, Y¹, and Y² satisfy the above (ii), specific examples of the ring forming a condensed cyclic structure by combining R⁴² and Y¹ together with the carbon atom to which R⁴² and Y¹ are bonded and the (thio)acetal include rings exemplified in the description of the ring R^{X}. Among these, the condensed cyclic structure formed by combining R⁴² and Y¹ preferably has a bridged aliphatic saturated hydrocarbon ring, a bridged aliphatic heteroring, or a polycyclic aromatic hydrocarbon ring, and more preferably has a bridged aliphatic saturated hydrocarbon ring or a bridged aliphatic heteroring.

Specific examples and preferable examples of the monovalent hydrocarbon group having 1 to 10 carbon atoms represented by R⁴³ are the same as the description of the (i).

Specific examples and preferable examples of the case where the group represented by Y² is a divalent linking group are the same as the specific examples and preferable examples of the Y¹.

When R⁴², R⁴³, Y¹, and Y² satisfy the above (iii), specific examples of the ring forming a spiro-ring structure by combining R⁴³ and Y¹ together with the carbon atom to which R⁴³ and Y¹ are bonded and the (thio)acetal include rings exemplified in the description of the ring R^{X}. Among these, the spiro-ring structure constituted by combining R⁴³ and Y¹ together with the (thio)acetal ring preferably has a bridged aliphatic saturated hydrocarbon ring, a bridged aliphatic heteroring, or a polycyclic aromatic hydrocarbon ring, and more preferably has a bridged aliphatic saturated hydrocarbon ring or a bridged aliphatic heteroring.

The alkanediyl group having 1 to 10 carbon atoms of R⁴² may be linear or branched. R⁴² preferably represents an alkanediyl group having 1 to 3 carbon atoms, and more preferably a methylene group from the viewpoint of ease of synthesis.

Specific examples and preferable examples of the case where the group represented by Y² is a divalent linking group are the same as the specific examples and preferable examples in the description of the Y¹.

In a case where the partial structure “—W¹—(COOH)_{b}” bonded to L¹ in the formula (1), wherein W¹ has a partial structure being the (b+1)-valent organic group, the “b” carboxy groups in “—W¹—(COOH)_{b}” may be bonded to the chain structure in W¹ or may be bonded to the ring. In terms of ability to more increase the effect of inhibiting development defects, when L¹ represents the group having the (thio)acetal ring, one or more carboxy groups in the “b” carboxy groups in the partial structure “—W¹—(COOH)_{b}”, wherein W¹ represents the (b+1)-valent organic group, are preferably bonded to the ring in W¹, and all the “b” carboxy groups are more preferably bonded to the ring in W¹. In a case where the partial structure “—W¹—(COOH)_{b}” bonded to L¹ in the formula (1), wherein W¹ has a partial structure being a single bond and L¹ represents the group having the (thio)acetal ring, the “b” carboxy groups in “—W¹—(COOH)_{b}” are preferably bonded to the ring R^{X} in L¹ or the (thio)acetal ring.

That is, when L¹ represents the group having the (thio)acetal ring, one or more partial structures “—W¹—(COOH)_{b}” bonded to L¹ in the formula (1) of the compound (B) preferably satisfy the following requirement (I) or (II).

(I) In one or more partial structures “—W¹—(COOH)_{b}” bonded to L¹ in the formula (1), W¹ represents a group having a cyclic structure, and one or more carboxy groups are bonded to the ring in W¹.

(II) In one or more partial structures “—W¹—(COOH)_{b}” bonded to L¹ in the formula (1), W¹ represents a single bond, L¹ in the formula (1) has a ring R^{X} forming a condensed cyclic structure or a spiro-ring structure together with the (thio)acetal ring in L¹, and a carboxy group is bonded to the ring R^{X} or the (thio)acetal ring.

When the compound (B) satisfies the requirement (I), the ring in W¹ to which the carboxy group is bonded is preferably an aliphatic hydrocarbon group, an aliphatic heteroring, or an aromatic ring, and more preferably a bridged aliphatic saturated hydrocarbon ring, a bridged aliphatic heteroring, or an aromatic ring. Among these, from the viewpoint of increase in the effect of inhibiting development defects, one or more carboxy groups in the formula (1) are preferably bonded to an aromatic ring in W¹, and all the carboxy groups in the formula (1) are more preferably bonded to an aromatic ring in W¹. The aromatic ring to which the carboxy group in the formula (1) is bonded is preferably an aromatic hydrocarbon ring. Examples of the aromatic hydrocarbon ring include a benzene ring, a naphthalene ring, and an anthracene ring. The aromatic hydrocarbon ring is preferably a benzene ring or a naphthalene ring, and more preferably a benzene ring.

When the compound (B) satisfies the requirement (II), preferable specific examples of the ring R^{X} in L¹ to which the carboxy group is bonded include the rings exemplified in the description of the ring R^{X} when W¹ represents a single bond.

When L¹ represents the group represented by the formula (L-2), W¹ preferably represents a single bond or a substituted or unsubstituted chain hydrocarbon group, more preferably a single bond, an alkanediyl group having 1 to 3 carbon atoms, or a fluoroalkanediyl group having 1 to 3 carbon atoms, and further preferably a single bond.

It is considered that the compound (B) having the carboxy group bonded to W¹ or L¹ in the formula (1) improves solubility of the compound (B) in an alkali developer liquid, and can consequently reduce an insoluble component in the exposed portion. As a result, LWR performance and development-defect inhibiting performance of the present composition are considered to be improved. Particularly, when the carboxy group in the compound (B) is directly bonded to the ring, it is considered that a degree of freedom of the carboxy group is reduced to inhibit aggregation of the compound (B), which can more reduce the insoluble component remained after development to further increase the effect of inhibiting development defects. In addition, when the present composition is applied for negative patterning, it is considered that the dissolution inhibiting effect in an organic-solvent developer liquid is increased to consequently improve CDU performance.

Examples of the monovalent hydrocarbon group represented by R¹, R², or R³ include groups same as the examples having a corresponding number of carbon atoms among the monovalent hydrocarbon groups exemplified in the description of R¹² in the formula (2). Among these, the monovalent hydrocarbon group having 1 to 10 carbon atoms represented by R¹, R², or R³ is preferably a monovalent chain hydrocarbon group having 1 to 10 carbon atoms or a monovalent alicyclic hydrocarbon group having 3 to 10 carbon atoms, more preferably a linear or branched alkyl group having 1 to 10 carbon atoms or a cycloalkyl group having 3 to 10 carbon atoms, further preferably an alkyl group having 1 to 3 carbon atoms, and furthermore preferably a methyl group, an ethyl group, or an isopropyl group.

Examples of the fluoroalkyl group represented by R¹, R², R³, or R^{f} include a linear or branched fluoroalkyl group having 1 to 10 carbon atoms. Specific examples thereof include a trifluoromethyl group, a 2,2,2-trifluoroethyl group, a pentafluoroethyl group, a 2,2,3,3,3-pentafluoropropyl group, a 1,1,1,3,3,3-hexafluoropropyl group, a heptafluoro-n-propyl group, a heptafluoro-i-propyl group, a nonafluoro-n-butyl group, a nonafluoro-i-butyl group, a nonafluoro-t-butyl group, a 2,2,3,3,4,4,5,5-octafluoro-n-pentyl group, a tridecafluoro-n-hexyl group, and a 5,5,5-trifluoro-1,1-diethylpentyl group. Among these, the fluoroalkyl group represented by R¹, R², R³, and R^{f} is preferably a linear or branched fluoroalkyl group having 1 to 3 carbon atoms, and more preferably a trifluoromethyl group.

R¹ and R² preferably represent a hydrogen atom, a fluorine atom, or a fluoroalkyl group having 1 to 3 carbon atoms, and more preferably a hydrogen atom, a fluorine atom, or a trifluoromethyl group. In terms of ability to increase acidity of the acid to be generated, R³ and R^{f} preferably represent both a fluorine atom or a trifluoroalkyl group. Specifically, R³ and R^{f} more preferably represent both a fluorine atom or a trifluoromethyl group, and further preferably both a fluorine atom. From the viewpoint of inhibiting diffusion of the acid generated by exposing the present composition, “a” preferably represents 0 to 5, more preferably 0 to 3, and further preferably 0 or 1. “b” preferably represents 1 or 2.

### —As for Cation

In the formula (1), M⁺ represents a monovalent cation. Examples of the monovalent cation represented by M⁺ include a sulfonium cation, an iodonium cation, and a quaternary ammonium cation. Among these, in terms of ability to form a high-quality resist film having high LWR performance and CDU performance, M⁺ preferably represents a sulfonium cation or an iodonium cation. Specific examples of the sulfonium cation include cations represented by the following formula (X-1), formula (X-2), formula (X-3), or formula (X-4). Specific examples of the iodonium cation include cations represented by the following formula (X-5) or formula (X-6).

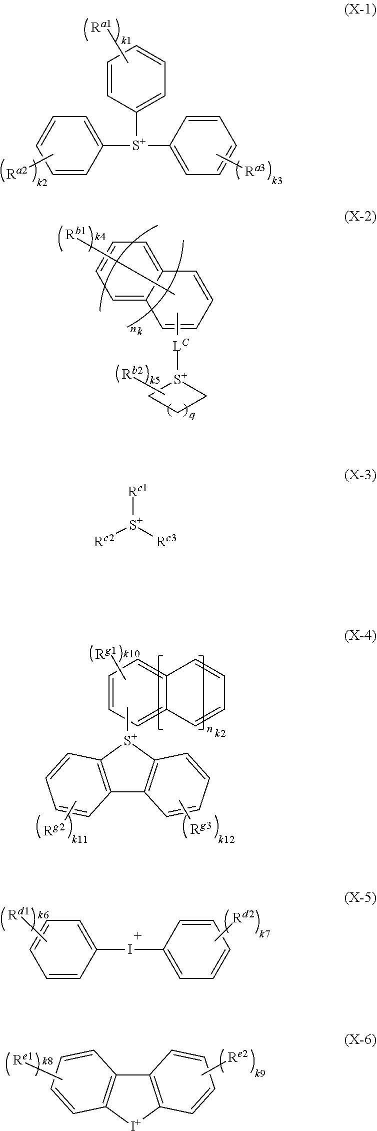

In the formula (X-1), R^{a1}, R^{a2}, and R^{a3} each independently represent a substituted or unsubstituted alkyl group, alkoxy group, alkylcarbonyloxy group, or cycloalkylcarbonyloxy group having 1 to 12 carbon atoms, a monocyclic or polycyclic cycloalkyl group having 3 to 12 carbon atoms, a monovalent aromatic hydrocarbon group having 6 to 12 carbon atoms, a hydroxy group, a halogen atom, —OSO₂—R^{P}, —SO₂—R^{Q}, or —S—R^{T}, or a cyclic structure constituted by combining two or more of R^{a1}, R^{a2}, and R^{a3}. This cyclic structure may have a heteroatom (such as an oxygen atom and a sulfur atom) between a carbon-carbon bond forming the skeleton. R^{P}, R^{Q}, and R^{T} each independently represent a substituted or unsubstituted alkyl group having 1 to 12 carbon atoms, a substituted or unsubstituted monovalent alicyclic hydrocarbon group having 5 to 25 carbon atoms, or a substituted or unsubstituted monovalent aromatic hydrocarbon group having 6 to 12 carbon atoms. k1, k2, and k3 each independently represent an integer of 0 to 5. When each of Rai to R^{a3} and R^{P}, R^{Q}, and R^{T} are plural, the plurality of R^{a1} to R^{a3} and R^{P}, and R^{P}, R^{Q}, and R^{T} are the same as or different from each other. When R^{a1}, R^{a2}, and R^{a3} have a substituent, this substituent may be a hydroxy group, a halogen atom, a carboxy group, a protected hydroxy group, a protected carboxy group, —OSO₂—R^{P}, —SO₂—R^{Q}, or —S—R^{T}.

In the formula (X-2), R^{b1} represents a substituted or unsubstituted alkyl group or alkoxy group having 1 to 20 carbon atoms, a substituted or unsubstituted acyl group having 2 to 8 carbon atoms, a substituted or unsubstituted monovalent aromatic hydrocarbon group having 6 to 8 carbon atoms, a halogen atom, or a hydroxy group. nₖ represents 0 or 1. When nₖ represents 0, k4 represents an integer of 0 to 4, and when nₖ represents 1, k4 represents an integer of 0 to 7. When R^{b1} is plural, the plurality of R^{b1} are the same or different, and the plurality of R^{b1} may represent a cyclic structure constituted by combining each other. R^{b2} represents a substituted or unsubstituted alkyl group having 1 to 7 carbon atoms or a substituted or unsubstituted monovalent aromatic hydrocarbon group having 6 or 7 carbon atoms. LC represents a single bond or a divalent linking group. k5 represents an integer of 0 to 4. When R^{b2} is plural, the plurality of R^{b2} are the same or different, and the plurality of R^{b2} may represent a cyclic structure constituted by combining each other. “q” represents an integer of 0 to 3. In the formula, the cyclic structure having S⁺ may have a heteroatom (such as an oxygen atom and a sulfur atom) between a carbon-carbon bond forming the skeleton.

In the formula (X-3), R^{c1}, R^{c2}, and R^{c3} each independently represent a substituted or unsubstituted alkyl group having 1 to 12 carbon atoms.

In the formula (X-4), R^{g1} represents a substituted or unsubstituted alkyl group or alkoxy group having 1 to 20 carbon atoms, a substituted or unsubstituted acyl group having 2 to 8 carbon atoms, or a substituted or unsubstituted aromatic hydrocarbon group having 6 to 8 carbon atoms, or a hydroxy group. nₖ₂ represents 0 or 1. When nₖ₂ represents 0, k10 represents an integer of 0 to 4, and when nₖ₂ represents 1, k10 represents an integer of 0 to 7. When R^{g1} is plural, the plurality of R^{g1} are the same or different, and the plurality of R^{g1} may represent a cyclic structure constituted by combining each other. R^{g2} and R^{g3} each independently represent a substituted or unsubstituted alkyl group, alkoxy group, or alkoxycarbonyl group having 1 to 12 carbon atoms, a substituted or unsubstituted monocyclic or polycyclic cycloalkyl group having 3 to 12 carbon atoms, a substituted or unsubstituted aromatic hydrocarbon group having 6 to 12 carbon atoms, a hydroxy group, a halogen atom, or a cyclic structure constituted by combining R^{g2} and R^{g3}. k1l and k12 each independently represent an integer of 0 to 4. When R^{g2} and R^{g3} are each plural, the plurality of R^{g2} and R^{g3} are the same as or different from each other.

In the formula (X-5), R^{d1} and R^{d2} each independently represent a substituted or unsubstituted alkyl group, alkoxy group, or alkoxycarbonyl group having 1 to 12 carbon atoms, a substituted or unsubstituted aromatic hydrocarbon group having 6 to 12 carbon atoms, a halogen atom, a halogenated alkyl group having 1 to 4 carbon atoms, a nitro group, or a cyclic structure constituted by combining two or more of these groups. k6 and k7 each independently represent an integer of 0 to 5. When each of R^{d1} and R^{d2} is plural, the plurality of R^{d1} and R^{d2} are the same as or different from each other.

In the formula (X-6), R^{e1} and R^{e2} each independently represent a halogen atom, a substituted or unsubstituted alkyl group having 1 to 12 carbon atoms, or a substituted or unsubstituted aromatic hydrocarbon group having 6 to 12 carbon atoms. k8 and k9 each independently represents an integer of 0 to 4. When each of R^{e1} and R^{e2} is plural, the plurality of R^{e1} and R^{e2} are the same as or different from each other.

Specific examples of the sulfonium cation and the iodonium cation represented by M⁺ include structures represented by the following formulae. The cations are not limited to these specific examples.

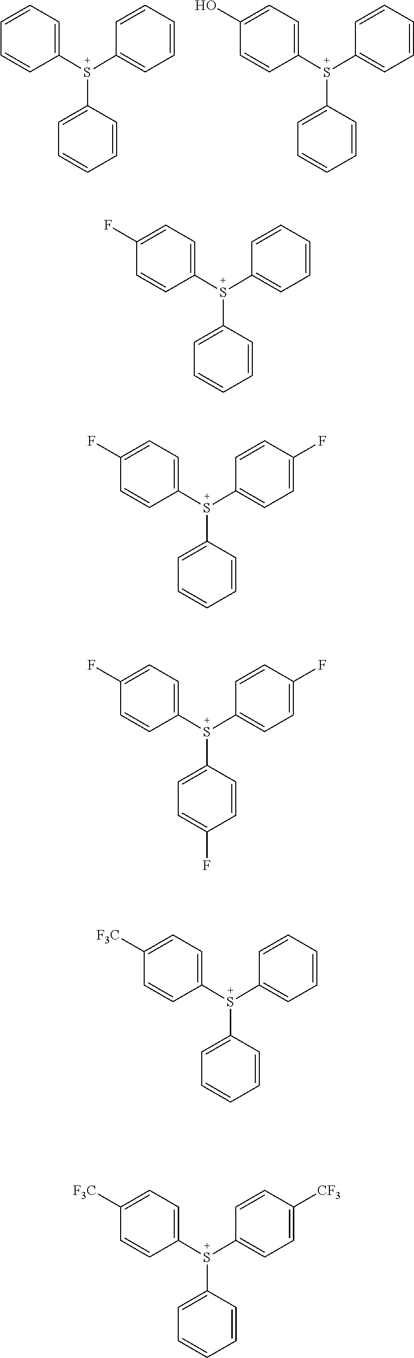

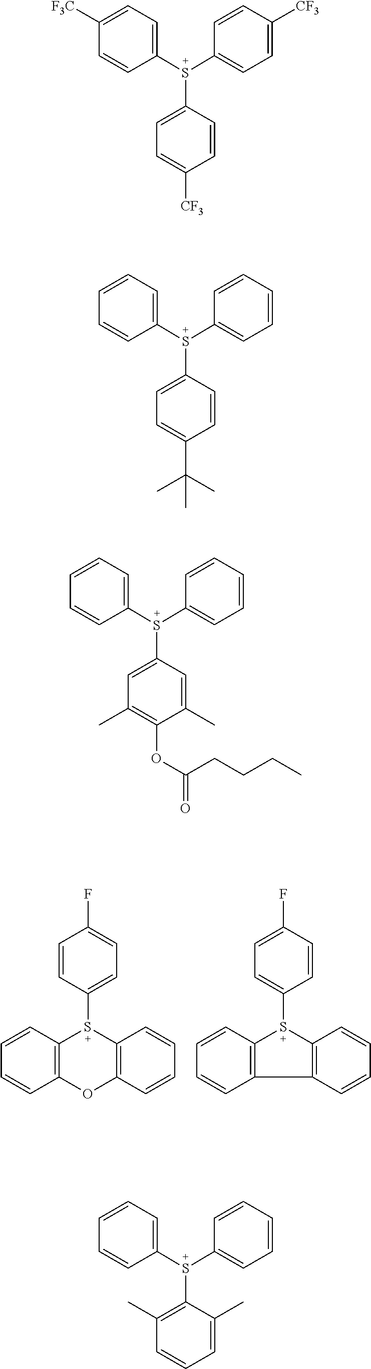

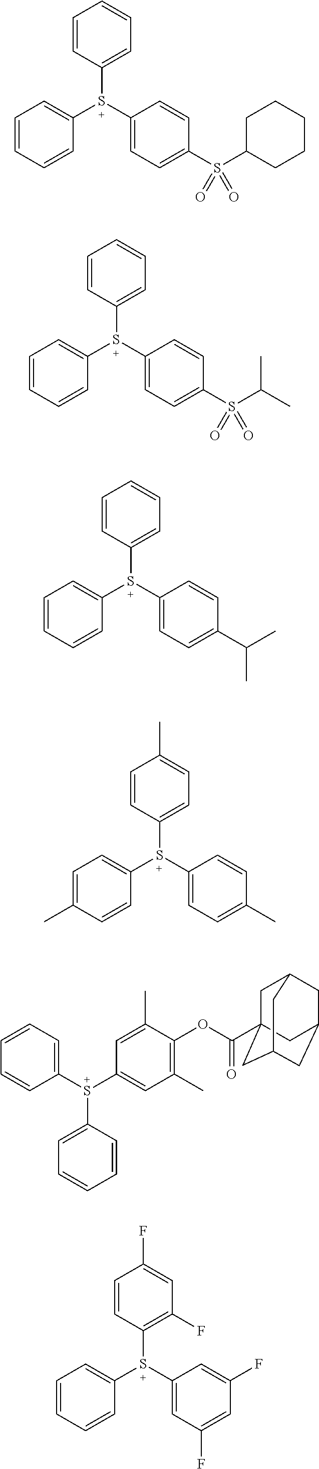

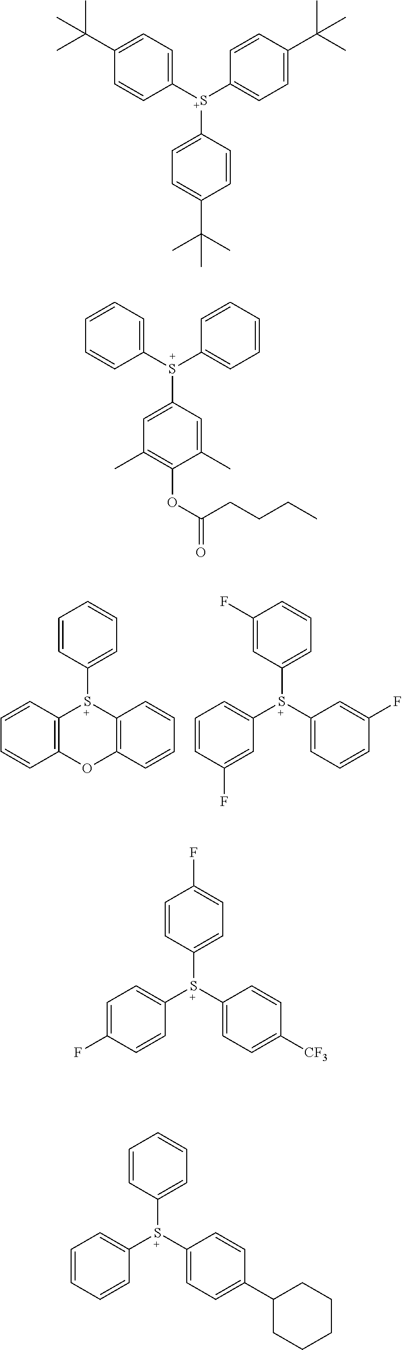

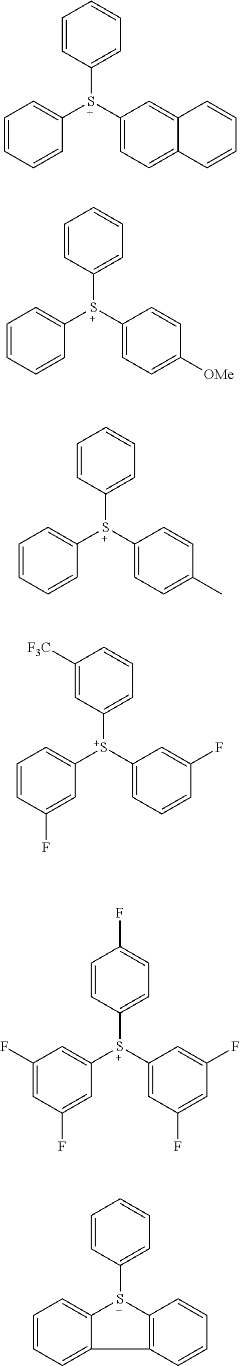

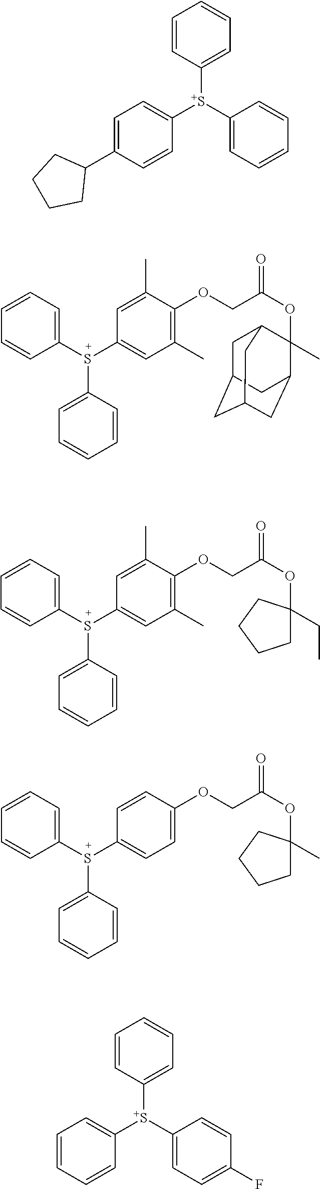

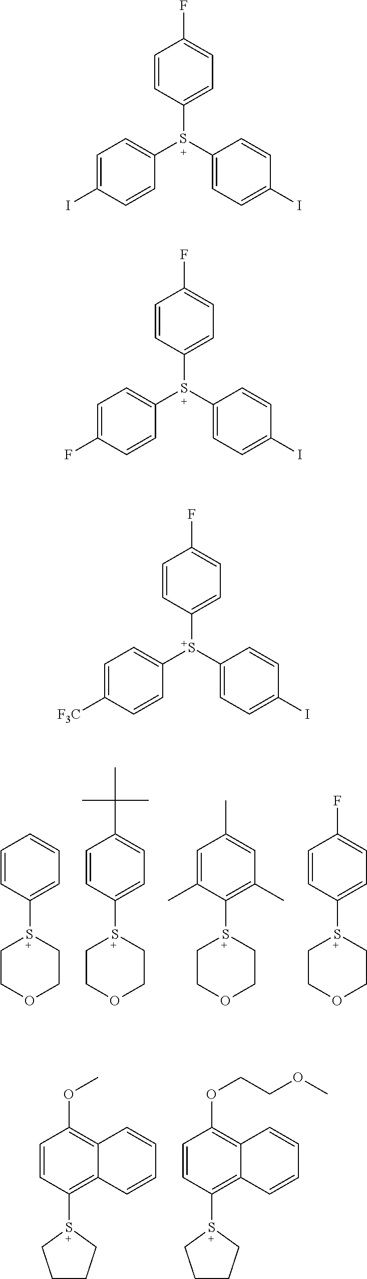

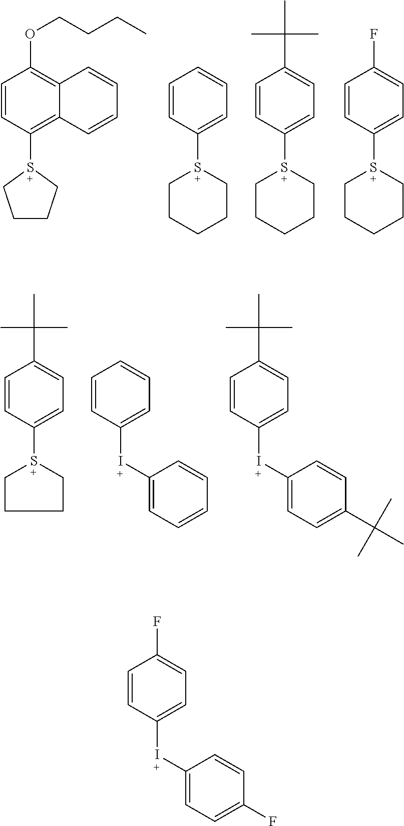

Among these, the compound (B) is preferably the sulfonium salt, and more preferably a triarylsulfonium salt. The compound (B) may be used singly, or may be used in combination of two or more thereof.

Specific examples of the compound (B) include compounds represented by each of the following formula (1-1) to formula (1-66).

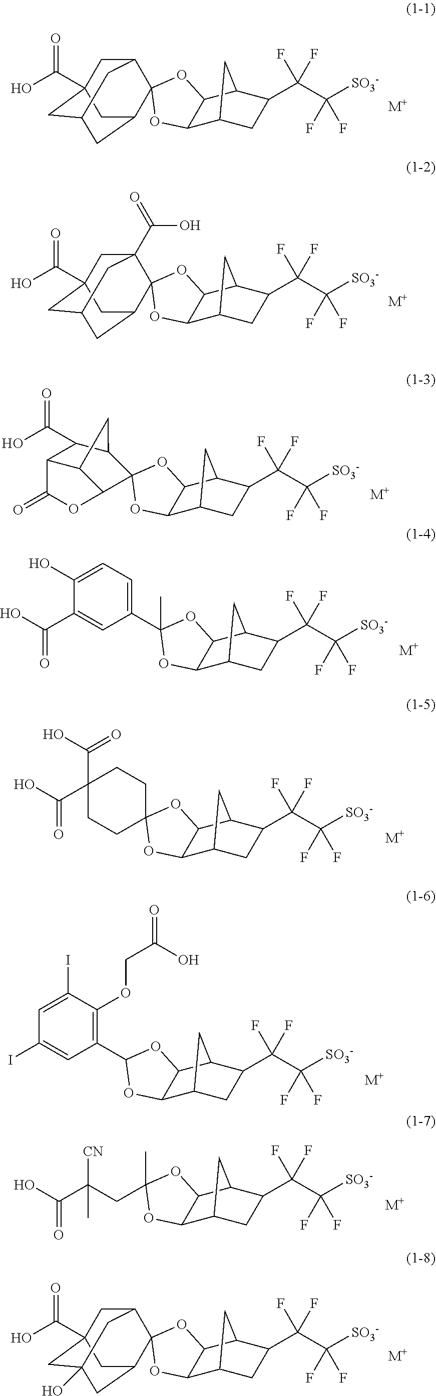

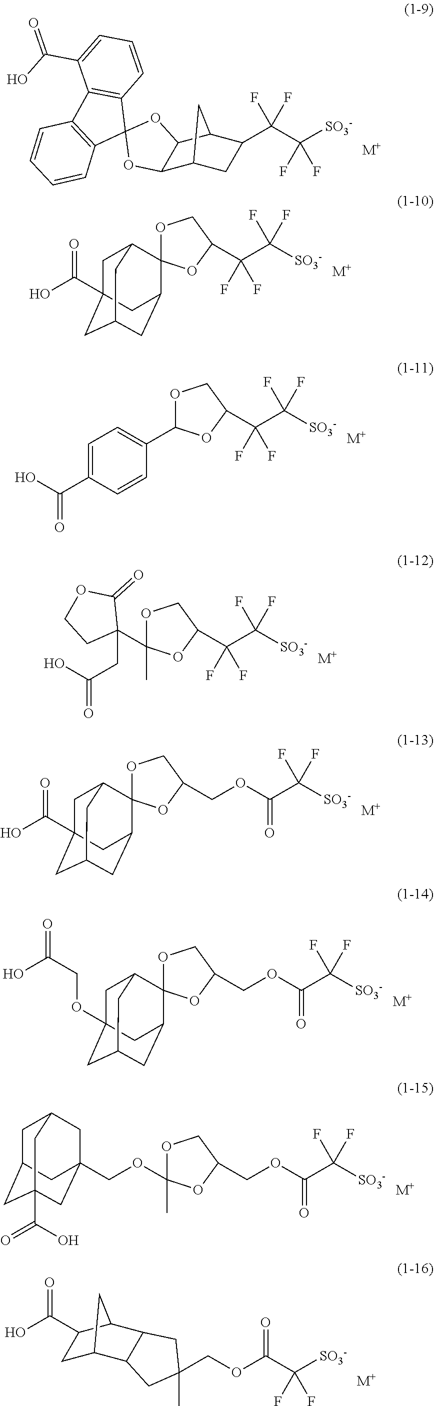

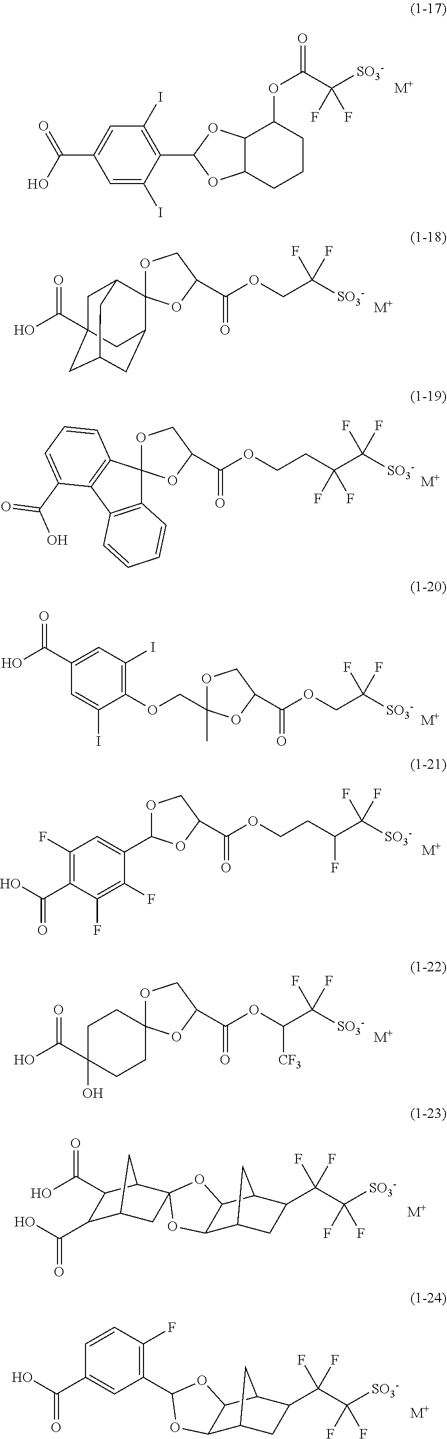

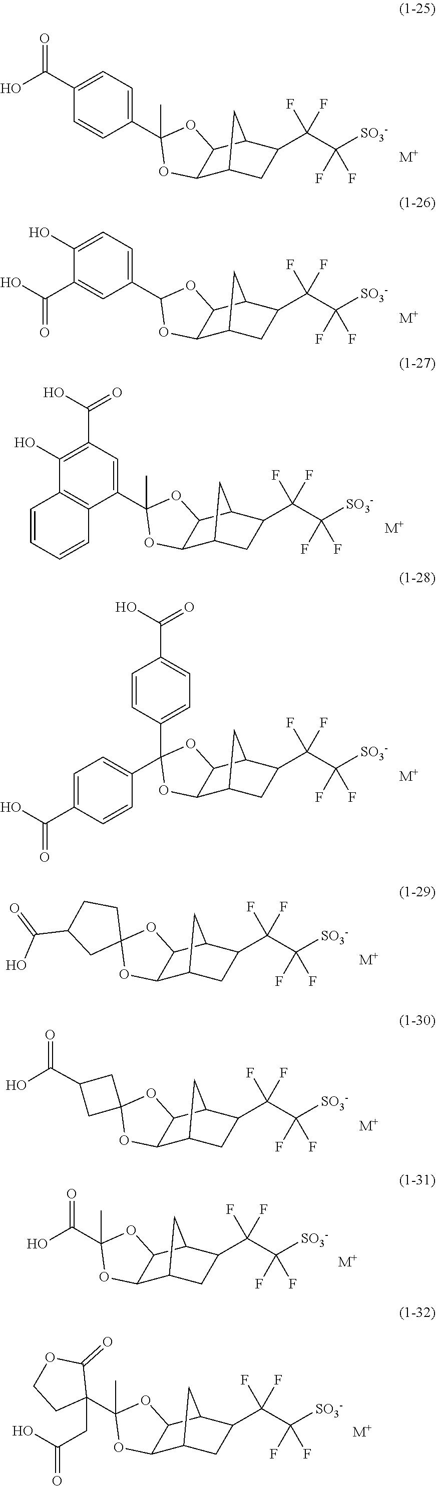

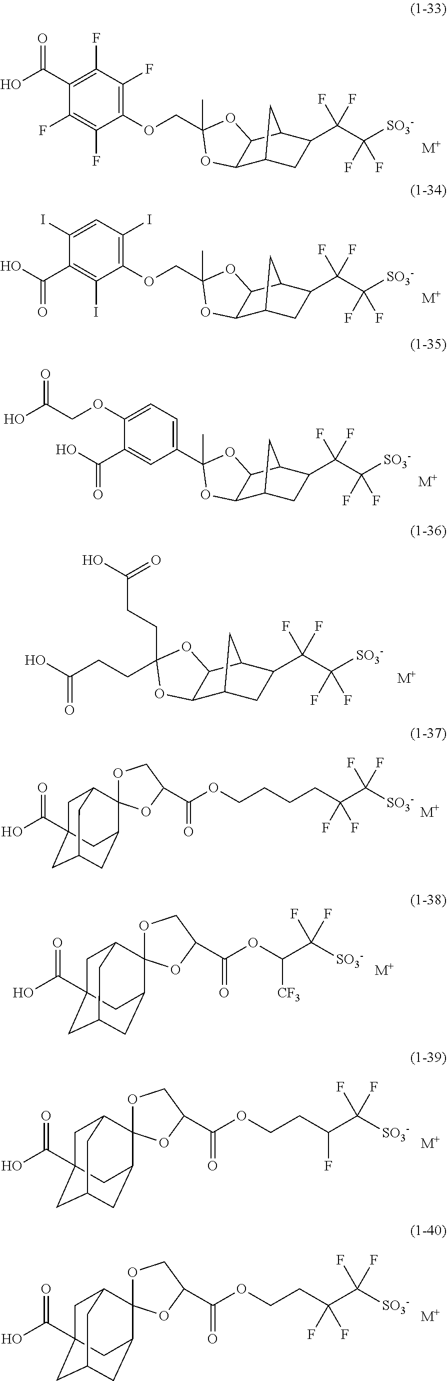

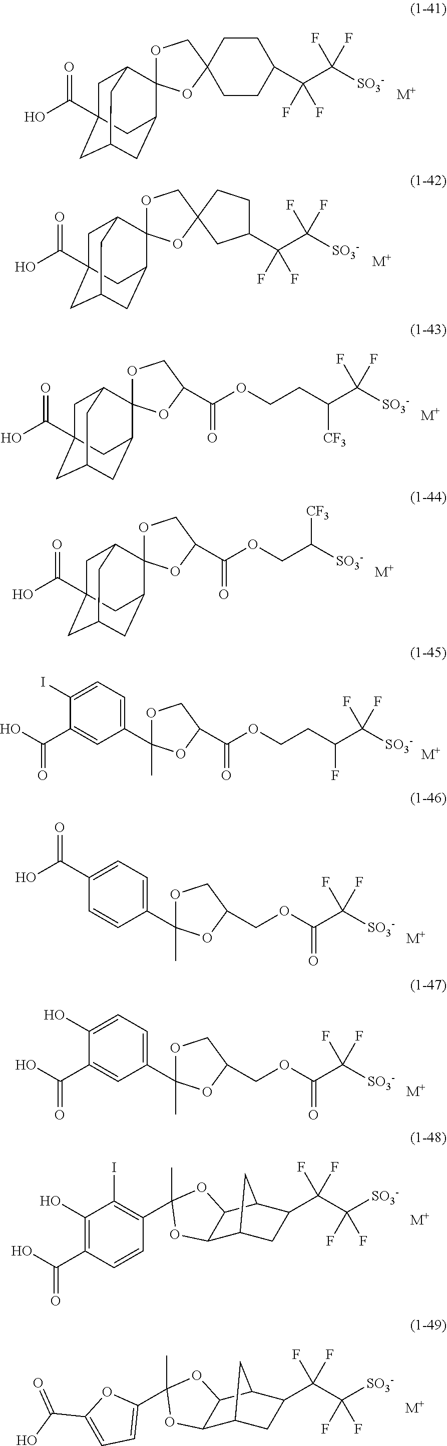

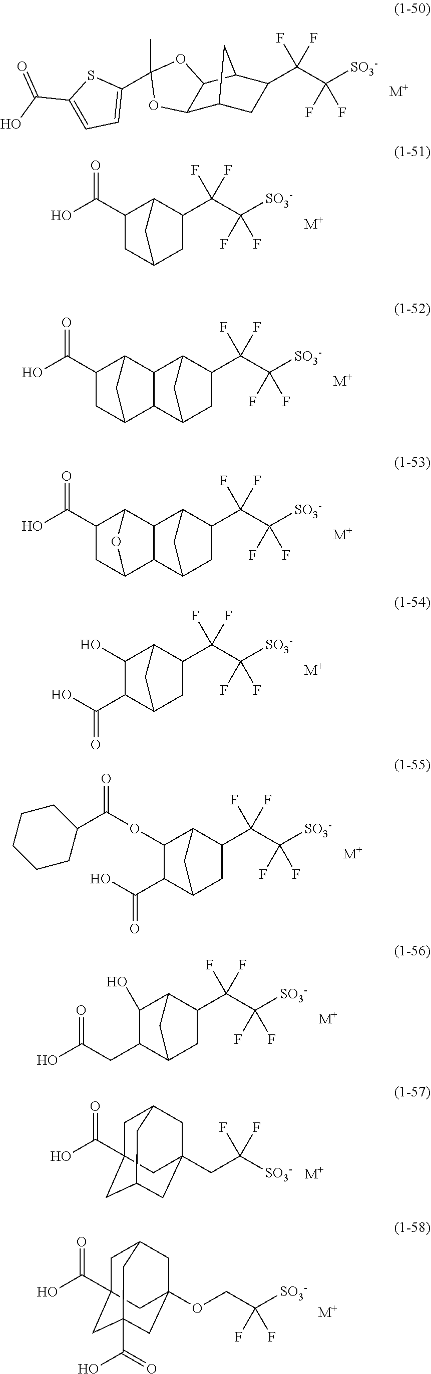

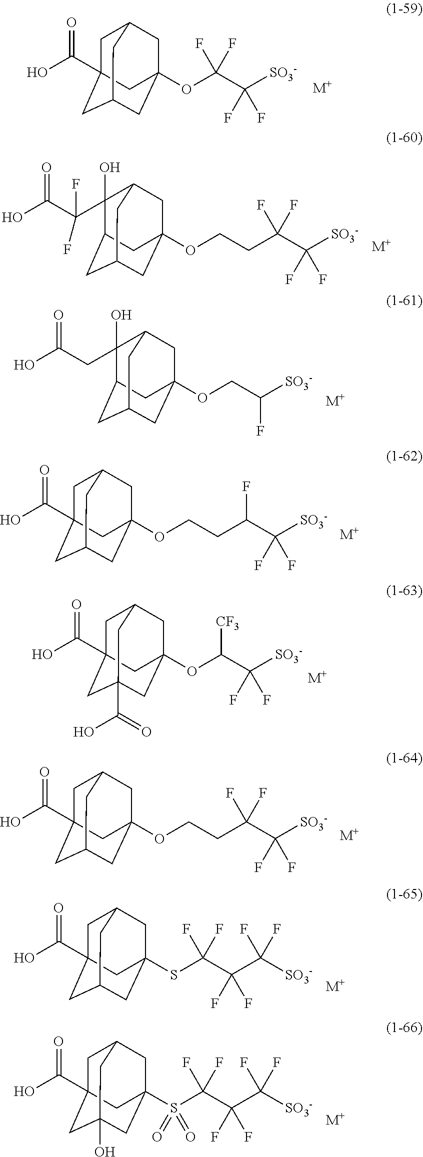

In the formula (1-1) to the formula (1-66), M⁺ represents the monovalent cation.

A content proportion of the compound (B) in the present composition is preferably 0.5 parts by mass or more, more preferably 1 part by mass or more, and further preferably 3 parts by mass or more relative to 100 parts by mass of the polymer (A). The content proportion of the compound (B) is preferably 45 parts by mass or less, more preferably 35 parts by mass or less, and further preferably 25 parts by mass or less relative to 100 parts by mass of the polymer (A). Setting the content proportion of the compound (B) to be within the above range can yield excellent LWR performance, CDU performance, and pattern profile while increasing sensitivity of the present composition, and can reduce development defects. The compound (B) may be used singly, or may be used in combination of two or more thereof.

### —Synthesis of Compound (B)

The compound (B) can be synthesized by appropriately combining usual methods of organic chemistry as described in Examples described later. For example, when L¹ in the formula (1) has the (thio)acetal ring, a diol product having a partial structure “—(CR¹R²)ₐ—CR^{f}R³—X⁵”, wherein X⁵ represents a halogen atom, is synthesized, then this diol product and a carboxy-group-containing compound having a structure corresponding to W¹ or L¹ are reacted in an appropriate solvent in the presence of a catalyst as necessary, then the obtained intermediate product is hydrolyzed and then reacted with a sulfonium chloride, a sulfonium bromide, etc. to yield the onium cation moiety.

Alternatively, the compound (B) can be synthesized by synthesizing a halogenated compound having the cyclic (thio)acetal structure and the partial structure “—(CR¹R²)ₐ—CR^{f}R³—X⁵” in the formula (1), hydrolyzing this halogenated compound and reacting with a sulfonium chloride, a sulfonium bromide, etc. to yield the onium cation moiety, and reacting the onium salt obtained from this reaction and a carboxy-group-containing compound having a structure corresponding to W¹ or L¹ in an appropriate solvent in the presence of a catalyst as necessary. The synthesis method of the compound (B) is not limited to the above.

### <Other Components>

The present composition may contain a component different from the polymer (A) and the compound (B) (hereinafter, also referred to as “other component”) together with the polymer (A) and the compound (B). Examples of the other component that the present composition may contain include an acid-diffusion inhibitor, a solvent, and a high-fluorine-content polymer.

### (Acid-Diffusion Inhibitor)

The acid-diffusion inhibitor is blended in the present composition for a purpose of inhibiting diffusion of the acid generated from the acid generator by exposure in the resist film to inhibit chemical reactions with the acid in the unexposed portion. Blending the acid-diffusion inhibitor in the present composition is preferable in terms of ability to more improve lithography properties of the present composition. Further, the acid-diffusion inhibitor can inhibit change in line width of a resist pattern due to variation of a holding time from exposure to development treatment, and the radiation-sensitive composition having excellent process stability can be obtained.

Examples of the acid-diffusion inhibitor include a nitrogen-containing compound and a photodegradable base. As the photodegradable base, a compound that generates a weaker acid (namely an acid with lower acidity) than the acid generated from the compound (B) by exposure can be used. Examples thereof include a compound that generates a weak acid (preferably a carboxylic acid), a sulfonic acid, and a sulfonamide by exposure. The degree of acidity can be evaluated with an acid dissociation constant (pKa). An acid dissociation constant of the acid generated from the photodegradable base is typically −3 or more, preferably −1≤pKa≤7, and more preferably 0≤pKa≤5.

### —Nitrogen-Containing Compound

Examples of the nitrogen-containing compound include a compound represented by the following formula (6) (hereinafter, also referred to as “nitrogen-containing compound (6A)”), a compound having two nitrogen atoms (hereinafter, also referred to as “nitrogen-containing compound (6B)”), a compound having three nitrogen atoms (hereinafter, also referred to as “nitrogen-containing compound (6C)”), an amide-group-containing compound, an urea compound, a nitrogen-containing heterocyclic compound, and a nitrogen-containing compound having an acid-releasable group.

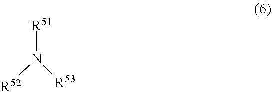

In the formula (6), R⁵¹, R⁵², and R⁵³ each independently represent a hydrogen atom, a substituted or unsubstituted alkyl group, a substituted or unsubstituted cycloalkyl group, a substituted or unsubstituted aryl group, or a substituted or unsubstituted aralkyl group.

As specific examples of the nitrogen-containing compound examples of the nitrogen-containing compound (6A) include: monoalkylamines such as n-hexylamine; dialkylamines such as di-n-butylamine; trialkylamines such as triethylamine and tri-n-pentylamine; and aromatic amines such as aniline and 2,6-diisopropylaniline.

Examples of the nitrogen-containing compound (6B) include ethylenediamine and N,N,N′,N′-tetramethylethylenediamine.

Examples of the nitrogen-containing compound (6C) include: polyamine compounds such as polyethyleneimine and polyarylamine; and polymers such as dimethylaminoethyl acrylamide.

Examples of the amide-group-containing compound include formamide, N-methylformamide, N,N-dimethylformamide, acetamide, N-methylacetamide, N,N-dimethylacetamide, propionamide, benzamide, pyrrolidone, and N-methylpyrrolidone.

Examples of the urea compound include urea, methylurea, 1,1-dimethylurea, 1,3-dimethylurea, 1,1,3,3-tetramethylurea, 1,3-diphenylurea, and tributylthiourea.

Examples of the nitrogen-containing heterocyclic compound include: pyridines such as pyridine and 2-methylpyridine; morpholines such as N-propylmorpholine and N-(undecan-1-ylcarbonyloxyethyl)morpholine; and pyrazine and pyrazole.

Examples of the nitrogen-containing compound having an acid-releasable group include N-t-butoxycarbonylpiperidine, N-t-butoxycarbonylimidazole, N-t-butoxycarbonylbenzimidazole, N-t-butoxycarbonyl-2-phenylbenzimidazole, N-(t-butoxycarbonyl)-di-n-octylamine, N-(t-butoxycarbonyl)diethanolamine, N-(t-butoxycarbonyl)dicyclohexylamine, N-(t-butoxycarbonyl)diphenylamine, N-t-butoxycarbonyl-4-hydroxypiperidine, and N-t-amyloxycarbonyl-4-hydroxypiperidine.

### —Photodegradable Base

The photodegradable base is a compound that generates an acid by radiation irradiation. The acid generated from the photodegradable base is an acid not inducing dissociation of the acid-releasable group under the normal condition. As the photodegradable base, an onium salt to generate a carboxylic acid, a sulfonic acid, or a sulfonamide by irradiation of radiation can be preferably used.

When the present composition contains the acid generator and the photodegradable base, the photodegradable base is a component to generate a weaker acid than the acid generated from the acid generator by exposure. The degree of acidity can be evaluated with an acid dissociation constant (pKa). The acid dissociation constant (pKa) of the acid generated from the photodegradable base is preferably −3 or more, more preferably −1≤pKa≤7, and further preferably 0≤pKa≤5.

The photodegradable base has basicity in the unexposed portion, and thus exhibits the effect of inhibiting acid diffusion. Meanwhile, the weak acid is generated from a proton generated by decomposition of the cation and an anion in the exposed portion, and thus the effect of inhibiting acid diffusion decreases. Therefore, in the resist film containing the photodegradable base, the acid-releasable group in the resist film is dissociated by efficient action of the generated acid in the exposed portion, and the components in the resist film does not change by acid in the unexposed portion. According to this mechanism, a difference in solubility between the exposed portion and the unexposed portion becomes more obvious.

Examples of the photodegradable base include an onium salt having a cation structure such as a sulfonium cation structure, an iodonium cation structure, and a quaternary ammonium cation structure. In terms of ability to form the resist film having higher LWR performance while keeping high sensitivity of the present composition, an onium salt having a sulfonium cation structure or an iodonium cation structure is preferably used as the photodegradable base, and specifically, at least one selected from the group consisting of a compound represented by the following formula (7A-1), a compound represented by the following formula (7A-2), a compound represented by the following formula (7B-1), and a compound represented by the following formula (7B-2).

(J^{a})⁺(E^{a})⁻ (7A-1)

In the formula (7A-1), (J^{a})⁺ represents a sulfonium cation. (E^{a})⁻ represents an anion represented by OH—, R^{α}—COO⁻, R^{α}—SO₃⁻, or R^{α}—N⁻(SO₂R^{f2}). R^{α} represents a monovalent hydrocarbon group, a monovalent group in which any methylene group in a monovalent hydrocarbon group is replaced by —O—, —CO—, —COO—, —O—CO—O—, —S—, —SO₂—, or —CONR^{β}— (hereinafter, also referred to as “group F^{A}”), or a monovalent group in which any hydrogen atom in a monovalent hydrocarbon group or the group F^{A} is replaced by a fluorine atom, an iodine atom, or a hydroxy group. R^{β} represents a hydrogen atom or a monovalent hydrocarbon group. R^{f2} represents a perfluoroalkyl group.

(J^{b})⁺R³¹-(E^{b})⁻ (7A-2)

In the formula (7A-2), (J^{b})⁺ represents a group having the sulfonium cation structure. (E^{b})⁻ represents *²—COO⁻, *²—SO₃⁻, or *²—N—(SO₂R^{f2}). “*²” represents a chemical bond. R^{f2} represents a perfluoroalkyl group. R³¹ represents a single bond, a divalent hydrocarbon group, or a divalent group in which any methylene group in a divalent hydrocarbon group is replaced by —O—, —CO—, —COO—, —O—CO—O—, —S—, —SO₂—, or —CONR^{β}— (hereinafter, also referred to as “group F^{B}”), or a divalent group in which any hydrogen atom in a divalent hydrocarbon group or the group F^{B} is replaced by a fluorine atom or a hydroxy group. R^{β} represents a hydrogen atom or a monovalent hydrocarbon group.

(U^{a})⁺(Q^{a})⁻ (7B-1)

In the formula (7B-1), (U^{a})⁺ represents an iodonium cation. (Q^{a})− represents an anion represented by OH⁻, R^{α}—COO⁻, R^{α}—SO₃⁻, or R^{α}—N⁻(SO₂R^{f2}). R^{α} each independently represents a monovalent hydrocarbon group, the monovalent group F^{A} in which any methylene group in a monovalent hydrocarbon group is replaced by —O—, —CO—, —COO—, —O—CO—O—, —S—, —SO₂—, or —CONR^{β}—, or a monovalent group in which any hydrogen atom in a monovalent hydrocarbon group or the group F^{A} is replaced by a fluorine atom or a hydroxy group. R^{β} represents a hydrogen atom or a monovalent hydrocarbon group. R^{f2} represents a perfluoroalkyl group.

(U^{b})⁺R³²-(Q^{b})⁻ (7B-2)

In the formula (7B-2), (U^{b})⁺ represents a group having the iodonium cation structure. (Q^{b})⁻ represents *²—COO⁻, *²—SO₃⁻, or *²—N⁻(SO₂R^{f2}). “*²” represents a chemical bond. R^{f2} represents a perfluoroalkyl group. R³² represents a single bond, a divalent hydrocarbon group, or the divalent group F^{B} in which any methylene group in a divalent hydrocarbon group is replaced by —O—, —CO—, —COO—, —O—CO—O—, —S—, —SO₂—, or —CONR^{β}—, or a divalent group in which any hydrogen atom in a divalent hydrocarbon group or the group F^{B} is replaced by a fluorine atom or a hydroxy group. R^{β} represents a hydrogen atom or a monovalent hydrocarbon group.

In the monovalent anion represented by (E^{a})⁻ in the formula (7A-1) and the monovalent anion represented by (Q^{a})⁻ in the formula (7B-1), examples of the monovalent hydrocarbon group represented by R^{α} include a monovalent chain hydrocarbon group having 1 to 10 carbon atoms, a monovalent alicyclic hydrocarbon group having 3 to 20 carbon atoms, and a monovalent aromatic hydrocarbon group having 6 to 20 carbon atoms. Specific examples thereof include the group exemplified as the monovalent hydrocarbon group having 1 to 20 carbon atoms represented by R¹² in the formula (2). Examples of the monovalent hydrocarbon group represented by R^{β} include a monovalent chain hydrocarbon group having 1 to 10 carbon atoms, a monovalent alicyclic hydrocarbon group having 3 to 12 carbon atoms, and a monovalent aromatic hydrocarbon group having 6 to 12 carbon atoms.

Examples of the perfluoroalkyl group represented by R^{f2} include a trifluoromethyl group, a pentafluoroethyl group, a 2,2,3,3,3-pentafluoropropyl group, a heptafluoro-n-propyl group, a heptafluoro-i-propyl group, a nonafluoro-n-butyl group, a nonafluoro-i-butyl group, and a nonafluoro-t-butyl group.

Specific examples of the anion represented by (E^{a})⁻ or (Q^{a})⁻ include structures represented by the following formula. The anion is not limited to these specific examples. Note that R²¹ and R²² in the following formula each independently represent an alkyl group having 1 to 20 carbon atoms.

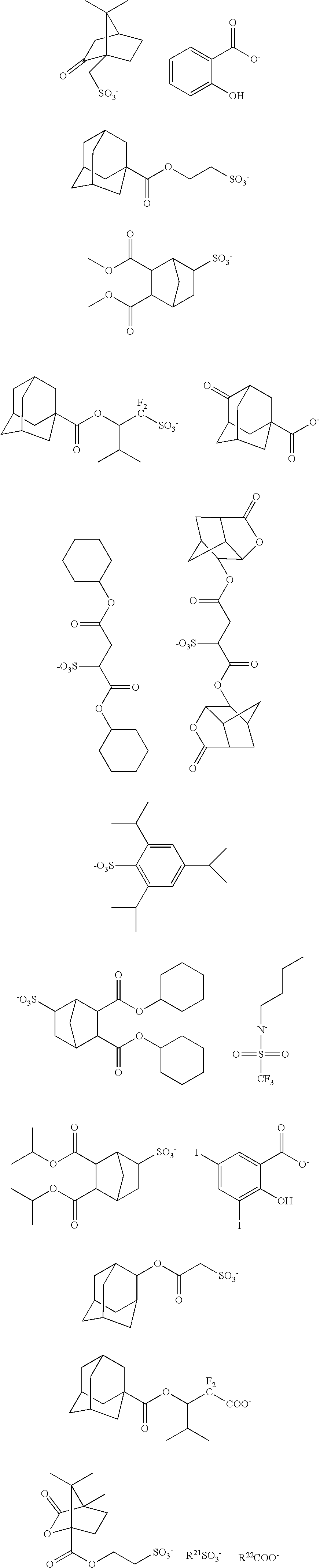

In the formula (7A-1), examples of the sulfonium cation represented by (J^{a})⁺ include sulfonium cations represented by the formula (X-1) to the formula (X-4). In the formula (7B-1), examples of the iodonium cation represented by (U^{a})⁺ include iodonium cations represented by the formula (X-5) or the formula (X-6).

In (E^{b})⁻ in the formula (7A-2) and (Q^{b})⁻ in the formula (7B-2), specific examples of the perfluoroalkyl group represented by R^{f2} include the groups exemplified as R^{f2} in the formula (7A-1) and the formula (7B-1).

Examples of the divalent hydrocarbon group represented by R³¹ in the formula (7A-2) and R³² in the formula (7B-2) include a group in which one hydrogen atom is removed from the group exemplified as the monovalent hydrocarbon group represented by R^{α}.

Specific examples of the partial structure represented by “—R³¹-(E^{b})⁻” in the formula (7A-2) and “—R³²-(Q^{b})⁻” in the formula (7B-2) include: a partial structure in which any hydrogen atom is removed from the structure exemplified as the specific examples of the anion represented by (E^{a})⁻ in the formula (7A-1) and (Q^{a})⁻ in the formula (7B-1); *²—COO−, *²—SO₃⁻, and *²—N—(SO₂R^{f2}).

Specific examples of the group represented by “-(J^{b})⁺” in the formula (7A-2) include a group in which any hydrogen atom is removed from the sulfonium cation represented by the formula (X-1) to the formula (X-4). Specific examples of the group represented by “—(U^{b})⁺” in the formula (7B-2) include a group in which any hydrogen atom is removed from the iodonium cation represented by the formula (X-5) or the formula (X-6).

Specific examples of the photodegradable base include compounds represented by the following formulae. The photodegradable base is not limited to these compounds.

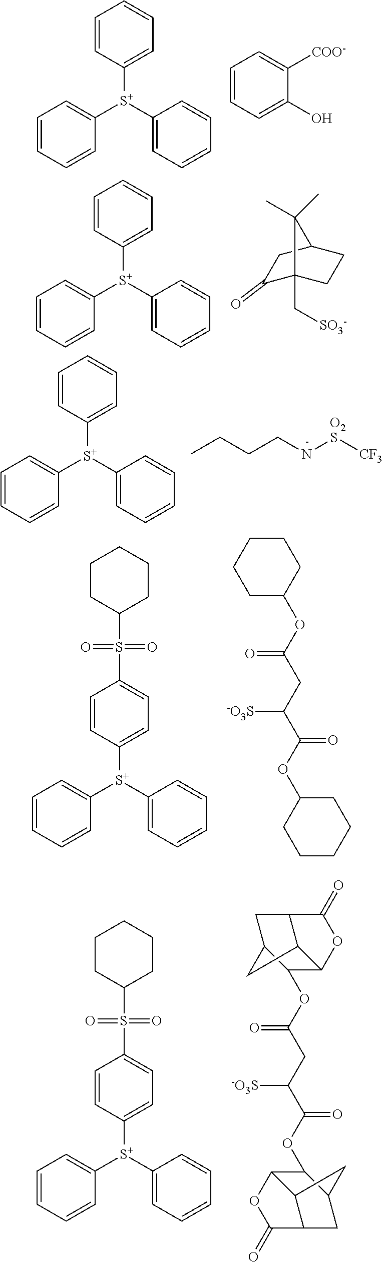

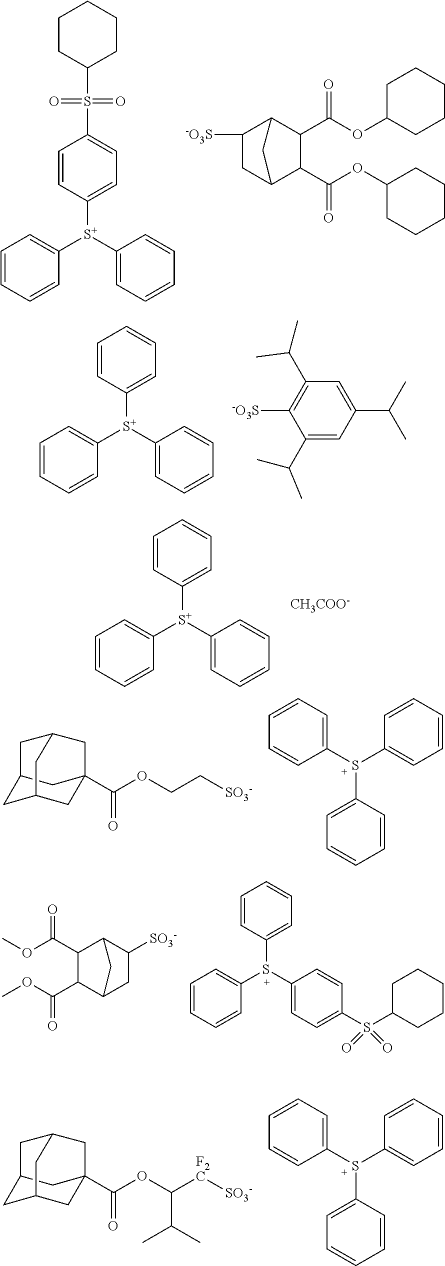

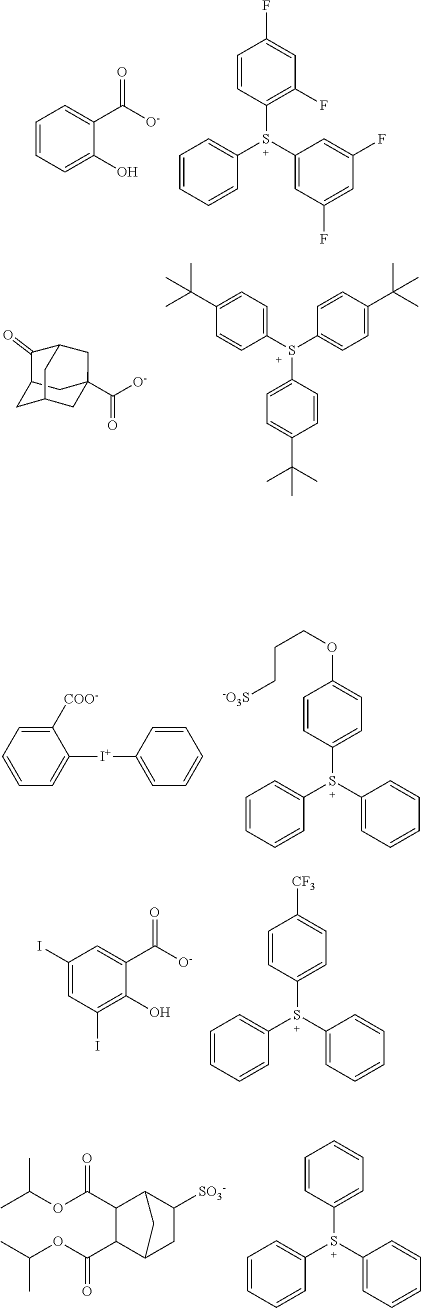

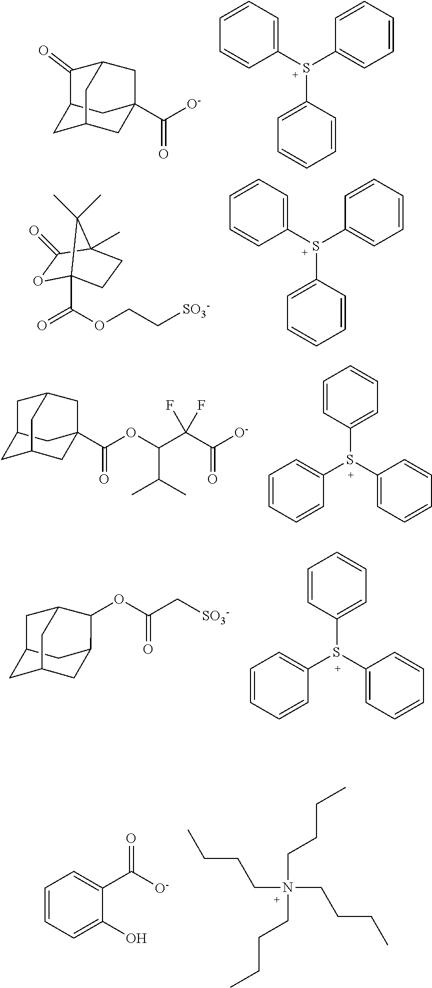

Among these, the photodegradable base used for preparing the present composition is preferably the sulfonium salt, and more preferably a triarylsulfonium salt. The photodegradable base may be used singly or in combination of two or more thereof.

When the present composition contains the acid-diffusion inhibitor, a content proportion of the acid-diffusion inhibitor in the present composition is preferably 0.1 part by mass or more, more preferably 1 part by mass or more, and further preferably 3 parts by mass or more relative to 100 parts by mass of the polymer (A). The content proportion of the acid-diffusion inhibitor is preferably 50 parts by mass or less, more preferably 45 parts by mass or less, and further preferably 40 parts by mass or less relative to 100 parts by mass of the polymer (A). Setting the content proportion of the acid-diffusion inhibitor to be within the above range is preferable in terms of ability to more improve LWR performance of the present composition. The acid-diffusion inhibitor may be used singly, or may be used in combination of two or more thereof.

### (Solvent)

The solvent may be any solvent that can dissolve or disperse the components blended in the present composition, and not particularly limited. Examples thereof include alcohols, ethers, ketones, amides, esters, and hydrocarbons.

Examples of the alcohols include: aliphatic monohydric alcohols having 1 to 18 carbon atoms such as 4-methyl-2-pentanol and n-hexanol; alicyclic monohydric alcohols having 3 to 18 carbon atoms such as cyclohexanol; polyhydric alcohols having 2 to 18 carbon atoms such as 1,2-propylene glycol; and polyhydric alcohol partial ethers having 3 to 19 carbon atoms such as propylene glycol monomethyl ether. Examples of the ethers include: dialkyl ethers such as diethyl ether, dipropyl ether, dibutyl ether, dipentyl ether, diisoamyl ether, dihexyl ether, and diheptyl ether; cyclic ethers such as tetrahydrofuran and tetrahydropyran; and aromatic-ring-containing ethers such as diphenyl ether and anisole.

Examples of the ketones include: chain ketones such as acetone, methyl ethyl ketone, methyl n-propyl ketone, methyl n-butyl ketone, diethyl ketone, methyl iso-butyl ketone, 2-heptanone, ethyl n-butyl ketone, methyl n-hexyl ketone, di-iso-butyl ketone, and trimethylnonanone; cyclic ketones such as cyclopentanone, cyclohexanone, cycloheptanone, cyclooctanone, and methylcyclohexanone; and 2,4-pentanedione, acetonylacetone, acetophenone, and diacetone alcohol. Examples of the amides include: cyclic amides such as N,N′-dimethylimidazolidinone and N-methylpyrrolidone; and chain amides such as N-methylformamide, N,N-dimethylformamide, N,N-diethylformamide, acetamide, N-methylacetamide, N,N-dimethylacetamide, and N-methylpropionamide.

Examples of the esters include: monocarboxylic acid esters such as n-butyl acetate and ethyl lactate; polyhydric alcohol carboxylates such as propylene glycol acetate; polyhydric alcohol partial ether carboxylates such as propylene glycol monomethyl ether acetate; polyvalent carboxylic acid diesters such as diethyl oxalate; carbonates such as dimethyl carbonate and diethyl carbonate; and cyclic esters such as γ-butyrolactone. Examples of the hydrocarbons: include aliphatic hydrocarbons having 5 to 12 carbon atoms such as n-pentane and n-hexane; and aromatic hydrocarbons having 6 to 16 carbon atoms such as toluene and xylene.

Among these, the solvent preferably contains at least one selected from the group consisting of the esters and ketones, and more preferably contains at least one selected from the group consisting of the polyhydric alcohol partial ether carboxylates and the cyclic ketones. The solvent may be used with one type or two or more types.

### (High-Fluorine-Content Polymer)

The high-fluorine-content polymer (hereinafter, also referred to as “polymer (F)”) is a polymer having a higher mass content rate of a fluorine atom than the polymer (A). When the present composition contains the polymer (F), the polymer (F) can be present unevenly in a surface layer of the resist film relative to the polymer (A), and hydrophobicity of the surface of the resist film can be increased in liquid-immersion exposure.

The fluorine atom content rate of the polymer (F) is not particularly limited as long as the content is higher than that of the polymer (A). The fluorine atom content rate of the polymer (F) is preferably 1 mass % or more, more preferably 2 mass % or more, further preferably 4 mass % or more, and particularly preferably 7 mass % or more. The fluorine atom content rate of the polymer (F) is preferably 60 mass % or less, more preferably 40 mass % or less, and further preferably 30 mass % or less. The fluorine atom content rate (mass %) of the polymer can be calculated from a structure of the polymer determined by ¹³C-NMR spectrum measurement etc.

Examples of a structural unit having a fluorine atom (hereinafter, referred to as “structural unit (f)”) in the polymer (F) include a structural unit (fa) and a structural unit (fb) described below. The polymer (F) may have one of the structural unit (fa) and the structural unit (fb), or may have both of the structural unit (fa) and the structural unit (fb) as the structural unit (f).

### —Structural Unit (fa)

The structural unit (fa) is a structural unit represented by the following formula (8-1). In the polymer (F), the fluorine atom content rate can be regulated by having the structural unit (fa).

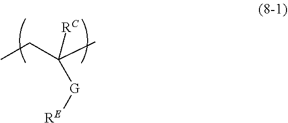

In the formula (8-1), R^{C} represents a hydrogen atom, a fluoro group, a methyl group, or a trifluoromethyl group. G represents a single bond, an oxygen atom, a sulfur atom, —COO—, —SO₂—O—NH—, —CONH—, or —O—CO—NH—. R^{E} represents a monovalent fluorinated chain hydrocarbon group having 1 to 20 carbon atoms or a monovalent fluorinated alicyclic hydrocarbon group having 3 to 20 carbon atoms.

In the formula (8-1), R^{C} preferably represents a hydrogen atom and a methyl group, and more preferably a methyl group from the viewpoint of copolymerization properties of a monomer to yield the structural unit (fa). G preferably represents a single bond or —COO—, and more preferably —COO— from the viewpoint of copolymerization properties of a monomer to yield the structural unit (fa).

Examples of the monovalent fluorinated chain hydrocarbon group having 1 to 20 carbon atoms represented by R^{E} include a group in which some or all of hydrogen atoms in a linear or branched alkyl group having 1 to 20 carbon atoms are replaced by a fluorine atom. Examples of the monovalent fluorinated alicyclic hydrocarbon group having 3 to 20 carbon atoms represented by R^{E} include a group in which some or all of hydrogen atoms in a monocyclic or polycyclic alicyclic hydrocarbon group having 3 to 20 carbon atoms are replaced by a fluorine atom. Among these, R^{E} preferably represents the monovalent fluorinated chain hydrocarbon group, more preferably a monovalent fluorinated alkyl group, and further preferably a 2,2,2-trifluoroethyl group, a 1,1,1,3,3,3-hexafluoropropyl group, or a 5,5,5-trifluoro-1,1-diethylpentyl group.

When the polymer (F) has the structural unit (fa), a content proportion of the structural unit (fa) is preferably 30 mol % or more, more preferably 40 mol % or more, and further preferably 50 mol % or more relative to all the structural units constituting the polymer (F). The content proportion of the structural unit (fa) is preferably 95 mol % or less, more preferably 90 mol % or less, and further preferably 85 mol % or less relative to all the structural units constituting the polymer (F). Setting the content proportion of the structural unit (fa) to be within the above range can more appropriately regulate the mass content rate of a fluorine atom in the polymer (F) to further enhance uneven distribution toward the surface layer of the resist film, which can more improve hydrophobicity of the resist film in liquid-immersion exposure.

### —Structural Unit (fb)

The structural unit (fb) is a structural unit represented by the following formula (8-2). The polymer (F) having the structural unit (fb) improves solubility in an alkali developer liquid, which can further inhibit occurrence of development defects.

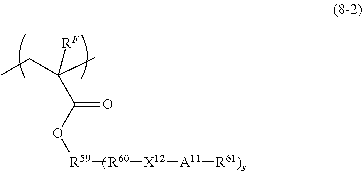

In the formula (8-2), R^{F} represents a hydrogen atom, a fluoro group, a methyl group, or a trifluoromethyl group. R⁵ represents a (s+1)-valent hydrocarbon group having 1 to 20 carbon atoms or a group in which an oxygen atom, a sulfur atom, —NR′—, a carbonyl group, —CO—O—, or —CO—NH— is bonded to a terminal on the R⁶⁰ side of the hydrocarbon group. R′ represents a hydrogen atom or a monovalent organic group. R⁶⁰ represents a single bond or a divalent organic group having 1 to 20 carbon atoms. X¹² represents a single bond, a hydrocarbon group having 1 to 20 carbon atoms, or a divalent fluorinated chain hydrocarbon group having 1 to 20 carbon atoms. A¹¹ represents an oxygen atom, —NR″—, —CO—O—*, or —SO₂—O—*. R″ represents a hydrogen atom or a monovalent hydrocarbon group having 1 to 10 carbon atoms. “*” represents a bonding position bonded to R⁶¹. R⁶¹ represents a hydrogen atom or a monovalent organic group having 1 to 30 carbon atoms. “s” represents an integer of 1 to 3. When “s” represents 2 or 3, a plurality of R⁶⁰, X¹², A¹¹, and R⁶¹ are each the same or different.

The structural unit (fb) is classified into a case of having an alkali-soluble group and a case of having a group to be dissociated by an action of an alkali to increase solubility in the alkali developer liquid (hereinafter, also simply referred to as “alkali-dissociable group”).

When the structural unit (fb) has the alkali-soluble group, R⁶¹ represents a hydrogen atom, A¹¹ represents an oxygen atom, —CO—O—*, or —SO₂—O—*. “*” represents a position bonded to R⁶¹. X¹² represents a single bond, a hydrocarbon group having 1 to 20 carbon atoms, or a divalent fluorinated chain hydrocarbon group having 1 to 20 carbon atoms. When A¹¹ represents an oxygen atom, X¹² represents a fluorinated hydrocarbon group having a fluorine atom or a fluoroalkyl group on the carbon atom to which A¹¹ is bonded. R⁶⁰ represents a single bond or a divalent organic group having 1 to 20 carbon atoms. When “s” represents 2 or 3, the plurality of R⁶⁰, X¹², A¹¹, and R⁶¹ are each same as or different from each other. The structural unit (fb) having the alkali-soluble group can increase compatibility with the alkali developer liquid to inhibit development defects.

When the structural unit (fb) has the alkali-dissociable group, R⁶¹ represents a monovalent organic group having 1 to 30 carbon atoms, and A¹¹ represents an oxygen atom, —NR″—, —CO—O—*, or —SO₂—O—*. “*” represents a position bonded to R⁶¹. X¹² represents a single bond or a divalent fluorinated chain hydrocarbon group having 1 to 20 carbon atoms. R⁶⁰ represents a single bond or a divalent organic group having 1 to 20 carbon atoms. When A¹¹ represents—CO—O—* or —SO₂—O—*, X¹² or R⁶¹ has a fluorine atom on a carbon atom bonded to A¹¹ or on a carbon atom adjacent thereto. When A¹¹ represents an oxygen atom, X¹² or R⁶⁰ represents a single bond, R⁵⁹ represents a structure in which a carbonyl group is bonded to a terminal on the R⁶⁰ side of the hydrocarbon group having 1 to 20 carbon atoms, and R⁶¹ represents an organic group having a fluorine atom. When “s” represents 2 or 3, the plurality of R⁶⁰, X¹², A¹¹, and R⁶¹ are each same as or different from each other. The structural unit (fb) having the alkali-soluble group allows the resist film surface to change from hydrophobic to hydrophilic in the alkali development step. This change can increase compatibility with the developer liquid to more efficiently inhibit development defects. As the structural unit (fb) having the alkali-dissociable group, it is particularly preferable that A¹¹ represent —CO—O—*, and R⁶¹ or X¹² or both thereof have a fluorine atom.

When the polymer (F) has the structural unit (fb), a content proportion of the structural unit (fb) is preferably 40 mol % or more, more preferably 50 mol % or more, and further preferably 60 mol % or more relative to all the structural units constituting the polymer (F). The content proportion of the structural unit (fb) is preferably 95 mol % or less, more preferably 90 mol % or less, and further preferably 85 mol % or less relative to all the structural units constituting the polymer (F). Setting the content proportion of the structural unit (fb) to be within the above range can more increase hydrophobicity of the resist film in liquid-immersion exposure.

The polymer (F) may have, other than the structural unit (fa) and the structural unit (fb), a structural unit (I) having an acid-releasable group or a structural unit having an alicyclic hydrocarbon structure represented by the following formula (9) (hereinafter, also referred to as “structural unit (G)”).

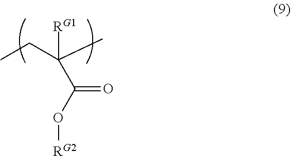

In the formula (9), R^{G1} represents a hydrogen atom, a fluorine atom, a methyl group, or a trifluoromethyl group. R^{G2} represents a monovalent alicyclic hydrocarbon group having 3 to 20 carbon atoms.

In the formula (9), examples of the monovalent alicyclic hydrocarbon group having 3 to 20 carbon atoms represented by R^{G2} include groups exemplified as the monovalent alicyclic hydrocarbon group having 3 to 20 carbon atoms represented by R¹³ to R¹⁵ in the formula (3).

When the polymer (F) has the structural unit represented by the formula (9), a content proportion of the structural unit is preferably 10 mol % or more, more preferably 20 mol % or more, and further preferably 30 mol % or more relative to all the structural units constituting the polymer (F). The content proportion of the structural unit represented by the formula (9) is preferably 70 mol % or less, more preferably 60 mol % or less, and further preferably 50 mol % or less relative to all the structural units constituting the polymer (F).

Mw of the polymer (F) by GPC is preferably 1,000 or more, more preferably 3,000 or more, and further preferably 4,000 or more. Mw of the polymer (F) is preferably 50,000 or less, more preferably 30,000 or less, and further preferably 20,000 or less. A molecular weight distribution (Mw/Mn) determined as a ratio between Mn and Mw by GPC of the polymer (F) is preferably 1 or more and 5 or less, and more preferably 1 or more and 3 or less.

When the present composition contains the polymer (F), a content proportion of the polymer (F) in the present composition is preferably 0.1 part by mass or more, more preferably 0.5 parts by mass of more, and further preferably 1 part by mass or more relative to 100 parts by mass of the polymer (A). The content proportion of the polymer (F) is preferably 10 parts by mass or less, more preferably 7 parts by mass or less, and further preferably 5 parts by mass or less relative to 100 parts by mass of the polymer (A). Note that the present composition may contain the polymer (F) singly, or may contain the polymer (F) in combination of two or more thereof.

### —Other Optional Components

The present composition may further contain a component different from the above polymer (A), the compound (B), the acid-diffusion inhibitor, the solvent, and the polymer (F) (hereinafter, also referred to as “other optional component”). Examples of the other optional component include an acid generator other than the compound (B), a surfactant, an alicyclic-skeleton-containing compound (for example, 1-adamantanecarboxylic acid, 2-adamantanone, t-butyl deoxycholate, etc.), a sensitizer, and an uneven-distribution enhancer. A content proportion of the other optional component in the present composition may be appropriately selected according to each component within a range not impairing the effect of the present disclosure.

When an acid generator other than the compound is blended in the present composition, a content proportion of the acid generator other than the component (B) is preferably 5 mass % or less, more preferably 3 mass % or less, further preferably 1 mass % or less, and particularly preferably 0.5 mass % or less relative to a total amount of the acid generator in the present composition from the viewpoint of obtaining the radiation-sensitive composition that can form the resist pattern having excellent LWR performance and pattern profile, and reduced development defects while exhibiting good sensitivity.

### <Method for Manufacturing Radiation-Sensitive Composition>

The present composition can be manufactured by, for example, mixing the polymer (A), the compound (B), and components such as the solvent as necessary at a desired ratio, and filtering the obtained mixture preferably by using a filter (for example, a filter with a pore diameter of about 0.2 μm). A solid-content concentration of the present composition is preferably 0.1 mass % or more, more preferably 0.5 mass % or more, and further preferably 1 mass % or more. The solid-content concentration of the present composition is preferably 50 mass % or less, more preferably 20 mass % or less, and further preferably 5 mass % or less. Setting the solid-content concentration of the present composition to be within the above range can provide good coatability to improve a shape of the resist pattern.

The present composition obtained as above can be used as a composition for positive-type pattern formation, which forms a pattern using an alkali developer liquid, or can be used as a composition for negative-type pattern formation, which forms a pattern using a developer liquid containing an organic solvent. Among these, the present composition is particularly suitable as a composition for positive-type pattern formation using an alkali developer liquid in terms of a higher effect exhibiting more excellent pattern rectangularity with development of the exposed resist film while exhibiting high sensitivity.

### <<Method for Forming Resist Pattern>>

A method for forming a resist pattern of the present disclosure includes: a step of applying the present composition on one surface of a substrate (hereinafter, also referred to as “applying step”); a step of exposing the resist film obtained in the applying step (hereinafter, also referred to as “exposing step”); and a step of developing the exposed resist film (hereinafter, also referred to as “developing step”). Examples of the pattern formed by the method for forming a resist pattern of the present disclosure include a line-and-space pattern and a hole pattern. The method for forming a resist pattern of the present disclosure uses the present composition to form the resist film, and thereby the resist pattern having good sensitivity and lithography properties and reduced development defects can be formed. Hereinafter, each step will be described.

### [Applying Step]

In the applying step, the present composition is applied on one surface of a substrate to form a resist film on the substrate. As the substrate on which the resist film is to be formed, conventional substrate may be used. Examples thereof include a silicon wafer, silicon dioxide, and a wafer coated with aluminum. For example, an organic or inorganic anti-reflective film, described in Japanese Patent No. H6-12452, Japanese Patent Laid-Open No. 559-93448, etc., may be formed on the substrate. Examples of a method for applying the present composition include spin coating, casting coating, and roll coating. After application, pre-baking (PB) to evaporate the solvent in the coating film may be performed. The temperature of PB is preferably 60° C. or higher, and more preferably 80° C. or higher. The temperature of PB is preferably 140° C. or lower, and more preferably 120° C. or lower. The time of PB is preferably 5 seconds or longer, and more preferably 10 seconds or longer. The time of PB is preferably 600 seconds or shorter, and more preferably 300 seconds or shorter. An average thickness of the resist film to be formed is preferably 10 to 1,000 nm, and more preferably 20 to 500 nm.

When liquid-immersion exposure is performed in the next exposing step, a protective film for liquid immersion insoluble in a liquid-immersion liquid may be further provided on the resist film formed with the present composition for a purpose of preventing direct contact between the liquid-immersion liquid and the resist film regardless of presence or absence of the hydrophobic polymer additive such as the polymer (F) in the present composition. As the protective film for liquid immersion, any one of a solvent-removable protective film, which is removed with a solvent before the developing step, (see Japanese Patent Laid-Open No. 2006-227632, for example) and a developer-liquid-removable protective film, which is removed simultaneously to development in the developing step (see WO 2005/069076 and WO 2006/035790, for example) may be used. From the viewpoint of throughput, the developer-liquid-removable protective film for liquid immersion is preferably used.

### [Exposing Step]

In the exposing step, the resist film obtained in the applying step is exposed. This exposure is performed by irradiating the resist film with radiation through a photomask, or through a liquid-immersion medium such as water in some cases. Examples of the radiation include: electromagnetic wave such as visible light ray, ultraviolet ray, far ultraviolet ray, extreme ultraviolet ray (EUV), X-ray, and γ-ray; and charged particle beam such as electron beam and α-ray, according to a line width of the target pattern. Among these, the radiation for irradiation of the resist film formed by using the present composition is preferably far ultraviolet ray, EUV, or electron beam, more preferably ArF excimer laser light (wavelength: 193 nm), KrF excimer laser light (wavelength: 248 nm), EUV, or electron beam, and further preferably ArF excimer laser light, EUV, or electron beam.

After the exposure, post exposure baking (PEB) is preferably performed to enhance dissociation of the acid-releasable group by the acid generated from the radiation-sensitive acid generator with exposure in the exposed portion of the resist film. This PEB can increase a difference in solubility in the developer liquid between the exposed portion and the unexposed portion. The temperature of PEB is preferably 50° C. or higher, and more preferably 80° C. or higher. The temperature of PEB is preferably 180° C. or lower, and more preferably 130° C. or lower. The time of PEB is preferably 5 seconds or longer, and more preferably 10 seconds or longer. The time of PEB is preferably 600 seconds or shorter, and more preferably 300 seconds or shorter.

### [Developing Step]

In the developing step, the exposed resist film is developed with the developer liquid. This development can form the desired resist pattern. The developer liquid may be an alkali developer liquid or an organic-solvent developer liquid. The developer liquid can be appropriately selected according to the target pattern (positive-type pattern or negative-type pattern).

Examples of the developer liquid for the alkali development include an alkali aqueous solution dissolving at least one of alkaline compounds such as sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethylamine, ethyldimethylamine, triethanolamine, tetramethylammonium hydroxide (TMAH), pyrrole, piperidine, choline, 1,8-diazabicyclo-[5.4.0]-7-undecene, and 1,5-diazabicyclo-[4.3.0]-5-nonene. Among these, a TMAH aqueous solution is preferable, and a 2.38 mass % TMAH aqueous solution is more preferable.

Examples of the developer liquid used for the organic-solvent development include organic solvents such as hydrocarbons, ethers, esters, ketones, and alcohols, and a solvent containing the organic solvent. Examples of the organic solvent include one or two or more of the solvent listed as the solvent that can be blended in the present composition. Among these, ethers, esters, and ketones are preferable. The ethers are preferably glycol ethers, and more preferably ethylene glycol monomethyl ether and propylene glycol monomethyl ether. The esters are preferably acetate esters, and more preferably n-butyl acetate or amyl acetate. The ketones are preferably chain ketones, and more preferably 2-heptanone. A content of the organic solvent in the developer liquid is preferably 80 mass % or more, more preferably 90 mass % or more, further preferably 95 mass % or more, and particularly preferably 99 mass % or more. Examples of a component other than the organic solvent in the developer liquid include water and silicone oil.

Examples of the development method include a method of immersing the substrate in a vessel filled with the developer liquid for a certain time (dip method), a method of lifting up the developer liquid to the substrate surface with a surface tension and leaving to stand the developer liquid for a certain time (puddle method), a method of spraying the developer liquid onto the substrate surface (spraying method), and a method of continuously discharging the developer liquid onto the substrate rotating at a certain speed while scanning the developer-liquid discharging nozzle at a certain speed (dynamic dispense method). After the development, the substrate is typically washed with a rinsing liquid such as water and an alcohol and then dried.

### EXAMPLES

Hereinafter, the present disclosure will be specifically described based on Examples, but the present disclosure is not limited to these Examples. Note that “parts” and “%” in the following examples are mass basis unless otherwise mentioned. Methods for measuring physical property values will be described below.

### [Weight-Average Molecular Weight (Mw), Number-Average Molecular Weight (Mn), and Degree of Dispersion (Mw/Mn)]

Mw and Mn of polymers were measured by gel permeation chromatography (GPC) with monodisperse polystyrene as a standard using GPC columns manufactured by Tosoh Corporation (G2000HXL: two columns, G3000HXL: one column, G4000HXL: one column) under an analysis condition of a flow rate: 1.0 mL/min, eluent solvent: tetrahydrofuran, sample concentration: 1.0 mass %, sample injection amount: 100 μL, column temperature: 40° C., detector: differential refractometer. The degree of dispersion (Mw/Mn) was calculated from measurement results of Mw and Mn.

### [¹³C-NMR Analysis]

¹³C-NMR analysis of the polymer was performed by using a nuclear magnetic resonance device (“JNM-Delta 400”, JEOL Ltd.).

[A] resin, [B] radiation-sensitive acid generator, [C] acid-diffusion inhibitor, [E] solvent, and [F] high-fluorine-content resin used for preparing a radiation-sensitive resin composition in each example are as follows.

### <[A] Resin and [F] High-Fluorine-Content Resin>

### —Synthesis of [A] Resin and [F] High-Fluorine Content Resin

Monomers used for synthesizing each of resins and high-fluorine-content resins will be described below. Note that, unless otherwise mentioned, “parts by mass” in the following Synthesis Examples means a value relative to a total mass of used monomers being 100 parts by mass, and “mol %” means a value relative to a total number of moles of the used monomers being 100 mol %.

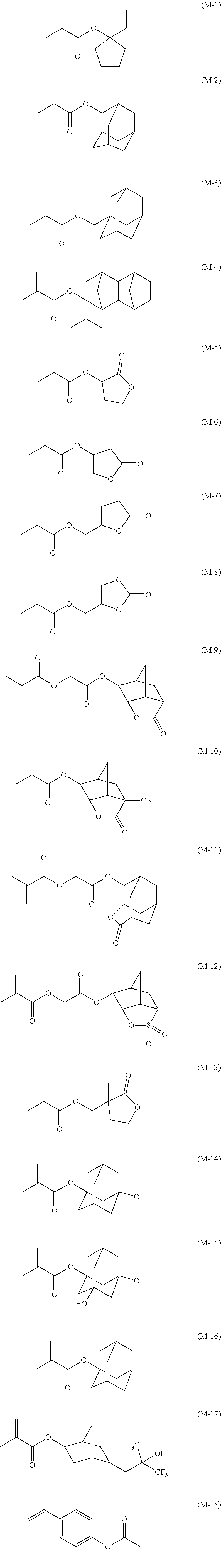

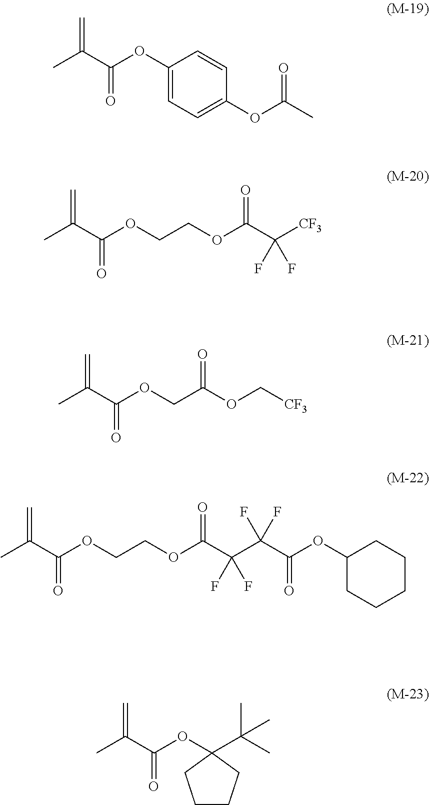

### Synthesis Example 1

### (Synthesis of Resin (A-1))

A monomer (M-1), a monomer (M-2), a monomer (M-5), a monomer (M-10), and a monomer (M-14) were dissolved in 2-butanone (200 parts by mass) at a mole ratio of 40/10/20/20/10 (mol %), and azobisisobutyronitrile (AIBN) (5 mol % relative to 100 mol % of a total of the used monomers) as an initiator was added to prepare a monomer solution. Into a reaction container, 2-butanone (100 parts by mass) was added, nitrogen purge was performed for 30 minutes, then an inside of the reaction container was set at 80° C., and the above monomer solution was added dropwise while stirring over 3 hours. The beginning of the dropwise addition was regarded as the beginning time of a polymerization reaction, and the polymerization reaction was performed for 6 hours. After the polymerization reaction was finished, the polymerization solution was cooled with water to 30° C. or lower. The cooled polymerization solution was poured into methanol (2,000 parts by mass), and the precipitated white powder was filtered. The filtered white powder was washed twice with methanol, filtered, and dried at 50° C. for 24 hours to obtain a white powdery resin (A-1) (yield: 85%). The resin (A-1) had Mw of 7,100 and Mw/Mn of 1.61. As a result of ¹³C-NMR analysis, content proportions of structural units derived from the monomer (M-1), the monomer (M-2), the monomer (M-5), the monomer (M-10), and the monomer (M-14) were respectively 40.3 mol %, 9.2 mol %, 20.5 mol %, 19.8 mol %, and 10.2 mol %.

### Synthesis Examples 2 to 13

### (Synthesis of Resin (A-2) to Resin (A-13))

A resin (A-2) to a resin (A-13) were synthesized in the same manner as in Synthesis Example 1 except that monomers with types at a blending ratio described in the following Table 1 were used. Content proportions (mol %) of structural units and physical property values (Mw and Mw/Mn) of the obtained resins are also shown in the following Table 1. Note that “-” in the following Table 1 indicates that the corresponding monomer is not used (the same applies to the following Tables).

TABLE 1

| | | Synthesis | | Synthesis | | Synthesis | | Synthesis | |
|---|---|---|---|---|---|---|---|---|---|
| | | Example 1 | | Example 2 | | Example 3 | | Example 4 | |
| [A] Resin | | A-1 | | A-2 | | A-3 | | A-4 | |
| Monomer | Type | M-1 | M-2 | M-1 | M-2 | M-1 | M-3 | M-1 | M-3 |
| providing | Amount of | 40 | 10 | 30 | 20 | 30 | 20 | 40 | 10 |
| structural | monomer | | | | | | | | |
| unit (I) | (mol %) | | | | | | | | |
| | Proportion | 40.3 | 9.2 | 30.2 | 19.2 | 31.0 | 19.6 | 40.5 | 10.3 |
| | of | | | | | | | | |
| | structural | | | | | | | | |
| | unit | | | | | | | | |
| | (mol %) | | | | | | | | |

| | | | | | | |
|---|---|---|---|---|---|---|
| Monomer | Type | M-5 | M-10 | M-9 | M-11 | M-12 |
| providing | Amount of | 20 | 20 | 50 | 50 | 50 |
| structural | monomer | | | | | |
| unit (II-1) | (mol %) | | | | | |
| | Proportion | 20.5 | 19.8 | 50.6 | 49.4 | 49.2 |
| | of | | | | | |
| | structural | | | | | |
| | unit | | | | | |
| | (mol %) | | | | | |

| | | | | | |
|---|---|---|---|---|---|
| Monomer | Type | M-14 | — | — | — |
| providing | Amount of | 10 | — | — | — |
| structural | monomer | | | | |
| unit (II-2) | (mol %) | | | | |
| | Proportion | 10.2 | — | — | — |
| | of | | | | |
| | structural | | | | |
| | unit | | | | |
| | (mol %) | | | | |

| | | | | |
|---|---|---|---|---|
| Mw | 7100 | 7700 | 7200 | 6800 |
| Mw/Mn | 1.61 | 1.51 | 1.59 | 1.61 |

| | | Synthesis | | Synthesis | | Synthesis |
|---|---|---|---|---|---|---|
| | | Example 5 | | Example 6 | | Example 7 |
| [A] Resin | | A-5 | | A-6 | | A-7 |
| Monomer | Type | M-1 | M-4 | M-1 | M-4 | M-1 |
| providing | Amount of | 40 | 10 | 40 | 10 | 50 |
| structural | monomer | | | | | |
| unit (I) | (mol %) | | | | | |
| | Proportion | 40.6 | 8.1 | 40.9 | 8.3 | 50.2 |
| | of | | | | | |
| | structural | | | | | |
| | unit | | | | | |
| | (mol %) | | | | | |

| Monomer | Type | M-13 | M-6 | M-9 | M-10 |
|---|---|---|---|---|---|
| providing | Amount of | 50 | 20 | 20 | 30 |
| structural | monomer | | | | |
| unit (II-1) | (mol %) | | | | |
| | Proportion | 51.3 | 20.5 | 20.3 | 29.6 |
| | of | | | | |
| | structural | | | | |
| | unit | | | | |
| | (mol %) | | | | |
| Monomer | Type | — | M-15 | | M-14 |
| providing | Amount of | — | 10 | | 20 |
| structural | monomer | | | | |
| unit (II-2) | (mol %) | | | | |
| | Proportion | — | 10.0 | | 20.2 |
| | of | | | | |
| | structural | | | | |
| | unit | | | | |
| | (mol %) | | | | |

| Mw | | 6900 | | 7500 | 7200 |
|---|---|---|---|---|---|
| Mw/Mn | | 1.44 | | 1.51 | 1.55 |
| | | Synthesis | | Synthesis | Synthesis |
| | | Example 8 | | Example 9 | Example 10 |
| [A] Resin | | A-8 | | A-9 | A-10 |
| Monomer | Type | M-1 | M-3 | M-1 | M-1 |
| providing | Amount of | 40 | 10 | 50 | 40 |
| structural | monomer | | | | |
| unit (I) | (mol %) | | | | |
| | Proportion | 40.0 | 10.1 | 50.3 | 40.2 |
| | of | | | | |
| | structural | | | | |
| | unit | | | | |
| | (mol %) | | | | |
| Monomer | Type | M-7 | M-11 | M-8 | M-9 |
| providing | Amount of | 20 | 20 | 50 | 60 |
| structural | monomer | | | | |
| unit (II-1) | (mol %) | | | | |
| | Proportion | 20.5 | 20.2 | 49.7 | 59.8 |
| | of | | | | |
| | structural | | | | |
| | unit | | | | |
| | (mol %) | | | | |
| Monomer | Type | M-15 | — | — | — |
| providing | Amount of | 10 | — | — | — |
| structural | monomer | | | | |
| unit (II-2) | (mol %) | | | | |
| | Proportion | 9.2 | — | — | — |
| | of | | | | |
| | structural | | | | |
| | unit | | | | |
| | (mol %) | | | | |

| Mw | | 7100 | 7000 | | 6700 | |
|---|---|---|---|---|---|---|
| Mw/Mn | | 1.62 | 1.51 | | 1.50 | |
| | | Synthesis | Synthesis | | Synthesis | |
| | | Example 11 | Example 12 | | Example 13 | |
| [A] Resin | | A-11 | A-12 | | A-13 | |
| Monomer | Type | M-2 | M-23 | M-2 | M-23 | M-3 |
| providing | Amount of | 40 | 40 | 10 | 40 | 10 |
| structural | monomer | | | | | |
| unit (I) | (mol %) | | | | | |
| | Proportion | 39.4 | 39.5 | 10.2 | 40.2 | 9.7 |
| | of | | | | | |
| | structural | | | | | |
| | unit | | | | | |
| | (mol %) | | | | | |
| Monomer | Type | M-10 | M-5 | M-12 | M-6 | M-11 |
| providing | Amount of | 60 | 20 | 25 | 20 | 25 |
| structural | monomer | | | | | |
| unit (II-1) | (mol %) | | | | | |
| | Proportion | 60.6 | 20.3 | 24.9 | 19.9 | 25.0 |
| | of | | | | | |
| | structural | | | | | |
| | unit | | | | | |
| | (mol %) | | | | | |

| Monomer | Type | — | M-14 | M-15 |
|---|---|---|---|---|
| providing | Amount of | — | 5 | 5 |
| structural | monomer | | | |
| unit (II-2) | (mol %) | | | |
| | Proportion | — | 5.1 | 5.2 |
| | of | | | |
| | structural | | | |
| | unit | | | |
| | (mol %) | | | |

| | | | |
|---|---|---|---|
| Mw | 7500 | 12300 | 16400 |
| Mw/Mn | 1.49 | 1.59 | 1.61 |

### Synthesis Example 14

### (Synthesis of Resin (A-14))

A monomer (M-1) and a monomer (M-18) were dissolved in 1-methoxy-2-propanol (200 parts by mass) at a mole ratio of 50/50 (mol %), and AIBN (5 mol %) as an initiator was added to prepare a monomer solution. Into a reaction container, 1-methoxy-2-propanol (100 parts by mass) was added, nitrogen purge was performed for 30 minutes, then an inside of the reaction container was set at 80° C., and the above monomer solution was added dropwise while stirring over 3 hours. The beginning of the dropwise addition was regarded as the beginning time of a polymerization reaction, and the polymerization reaction was performed for 6 hours. After the polymerization reaction was finished, the polymerization solution was cooled with water to 30° C. or lower. The cooled polymerization solution was poured into hexane (2,000 parts by mass), and the precipitated white powder was filtered. The filtered white powder was washed twice with hexane, filtered, and dissolved in 1-methoxy-2-propanol (300 parts by mass). Then, methanol (500 parts by mass), triethylamine (50 parts by mass), and ultrapure water (10 parts by mass) were added, and a hydrolysis reaction was performed at 70° C. for 6 hours while stirring. After the reaction was finished, the residual solvent was distilled off, the obtained solid was dissolved in acetone (100 parts by mass), and the solution was added dropwise into water (500 parts by mass) to solidify the resin. The obtained solid was filtered, and dried at 50° C. for 13 hours to obtain a white powdery resin (A-14) (yield: 81%). The resin (A-14) had Mw of 5,500 and Mw/Mn of 1.62. As a result of ¹³C-NMR analysis, content proportions of structural units derived from the monomer (M-1) and the monomer (M-18) were respectively 50.2 mol % and 49.8 mol %.

### Synthesis Example 15 to Synthesis Example 18

### (Synthesis of Resin (A-15) to Resin (A-18))

A resin (A-15) to a resin (A-18) were synthesized in the same manner as in Synthesis Example 14 except that monomers with types at a blending ratio described in the following Table 2 were used. In a monomer to yield the structural unit (III), all alkali-dissociable groups were hydrolyzed to form phenolic hydroxy groups. Content proportions (mol %) of structural units and physical property values (Mw and Mw/Mn) of the obtained resins are also shown in the following Table 2.

TABLE 2

| | | Syn- | Syn- | Syn- | Syn- | Syn- |
|---|---|---|---|---|---|---|
| | | thesis | thesis | thesis | thesis | thesis |
| | | Example | Example | Example | Example | Example |
| | | 14 | 15 | 16 | 17 | 18 |
| [A] Resin | | A-14 | A-15 | A-16 | A-17 | A-18 |
| Monomer | Type | M-1 | M-3 | M-2 | M-1 | M-23 |
| providing | Amount of | 50 | 50 | 50 | 55 | 55 |
| structural | monomer | | | | | |
| unit (I) | (mol %) | | | | | |
| | Proportion | 50.2 | 46.6 | 48.1 | 55.7 | 55.1 |
| | of | | | | | |
| | structural | | | | | |
| | unit | | | | | |
| | (mol %) | | | | | |
| Monomer | Type | — | M-14 | M-17 | M-17 | M-15 |
| providing | Amount of | — | 10 | 20 | 15 | 5 |
| structural | monomer | | | | | |
| unit | (mol %) | | | | | |
| (II-2) | Proportion | — | 11.1 | 21.3 | 15.1 | 4.9 |
| | of | | | | | |
| | structural | | | | | |
| | unit | | | | | |
| | (mol %) | | | | | |
| Monomer | Type | M-18 | M-19 | M-18 | M-19 | M-18 |
| providing | Amount of | 50 | 40 | 30 | 30 | 40 |
| structural | monomer | | | | | |
| unit (III) | (mol %) | | | | | |
| | Proportion | 49.8 | 42.3 | 30.6 | 29.2 | 40.0 |
| | of | | | | | |
| | structural | | | | | |
| | unit | | | | | |
| | (mol %) | | | | | |

| | | | | | |
|---|---|---|---|---|---|
| Mw | 5500 | 5600 | 5100 | 6100 | 13100 |
| Mw/Mn | 1.62 | 1.55 | 1.59 | 1.50 | 1.56 |

### Synthesis Example 19

### (Synthesis of High-Fluorine-Content Resin (F-1))

A monomer (M-1) and a monomer (M-20) were dissolved in 2-butanone (200 parts by mass) at a mole ratio of 20/80 (molo), and AIBN (4 molo) as an initiator was added to prepare a monomer solution. Into a reaction container, 2-butanone (100 parts by mass) was added, nitrogen purge was performed for 30 minutes, then an inside of the reaction container was set at 80° C., and the above monomer solution was added dropwise while stirring over 3 hours. The beginning of the dropwise addition was regarded as the beginning time of a polymerization reaction, and the polymerization reaction was performed for 6 hours. After the polymerization reaction was finished, the polymerization solution was cooled with water to 30° C. or lower. The solvent was replaced with acetonitrile (400 parts by mass), then hexane (100 parts by mass) was added and stirred, and the acetonitrile layer was recovered. This operation was repeated three times. The solvent is replaced with propylene glycol monomethyl ether acetate to obtain a solution of a high-fluorine-content resin (F-1) (yield: 75%). The high-fluorine-content resin (F-1) had Mw of 6,200 and Mw/Mn of 1.77. As a result of ¹³C-NMR analysis, content proportions of structural units derived from (M-1) and (M-20) were respectively 19.5 mol % and 80.5 mol %.

### Synthesis Example 20 to Synthesis Example 23

### (Synthesis of High-Fluorine-Content Resin (F-2) to High-Fluorine-Content Resin (F-5))

A high-fluorine-content resin (F-2) to a high-fluorine-content resin (F-5) were synthesized in the same manner as in Synthesis Example 19 except that monomers with types at a blending ratio described in the following Table 3 were used. Content proportions (mol %) of structural units and physical property values (Mw and Mw/Mn) of the obtained high-fluorine-content resins are also shown in the following Table 3.

TABLE 3

| | | Syn- | Syn- | Syn- | Syn- | Syn- |
|---|---|---|---|---|---|---|
| | | thesis | thesis | thesis | thesis | thesis |
| | | Example | Example | Example | Example | Example |
| | | 19 | 20 | 21 | 22 | 23 |
| [F] High-Fluorine- | | F-1 | F-2 | F-3 | F-4 | F-5 |
| Content-Resin | | | | | | |
| Monomer | Type | M-20 | M-21 | M-22 | M-22 | M-20 |
| providing | Amount of | 80 | 80 | 60 | 60 | 60 |
| structural | monomer | | | | | |
| unit (f) | (mol %) | | | | | |
| | Proportion | 80.5 | 80.2 | 61.3 | 60.2 | 60.0 |
| | of | | | | | |
| | structural | | | | | |
| | unit | | | | | |
| | (mol %) | | | | | |
| Monomer | Type | M-1 | M-1 | — | M-2 | M-3 |
| providing | Amount of | 20 | 20 | — | 20 | 10 |
| structural | monomer | | | | | |
| unit (I) | (mol %) | | | | | |
| | Proportion | 19.5 | 19.8 | — | 19.4 | 10.1 |
| | of | | | | | |
| | structural | | | | | |
| | unit | | | | | |
| | (mol %) | | | | | |
| Monomer | Type | — | — | — | M-14 | M-17 |
| providing | Amount of | — | — | — | 20 | 30 |
| structural | monomer | | | | | |
| unit | (mol %) | | | | | |
| (II-2) | Proportion | — | — | — | 20.4 | 29.9 |
| | of | | | | | |
| | structural | | | | | |
| | unit | | | | | |
| | (mol %) | | | | | |
| Monomer | Type | — | — | M-16 | — | — |
| providing | Amount of | — | — | 40 | — | — |
| other | monomer | | | | | |
| structural | (mol %) | | | | | |
| unit | Proportion | — | — | 38.7 | — | — |
| | of | | | | | |
| | structural | | | | | |
| | unit | | | | | |
| | (mol %) | | | | | |

| | | | | | |
|---|---|---|---|---|---|
| Mw | 6200 | 7100 | 6900 | 7300 | 6700 |
| Mw/Mn | 1.77 | 1.82 | 1.91 | 1.88 | 1.87 |

### <[B] Radiation-Sensitive Acid Generator>

### [Synthesis of Radiation-Sensitive Acid Generator (B)]

### Synthesis Example 24

### (Synthesis of Compound (B-1))

A compound (B-1) was synthesized according to the following synthesis scheme.

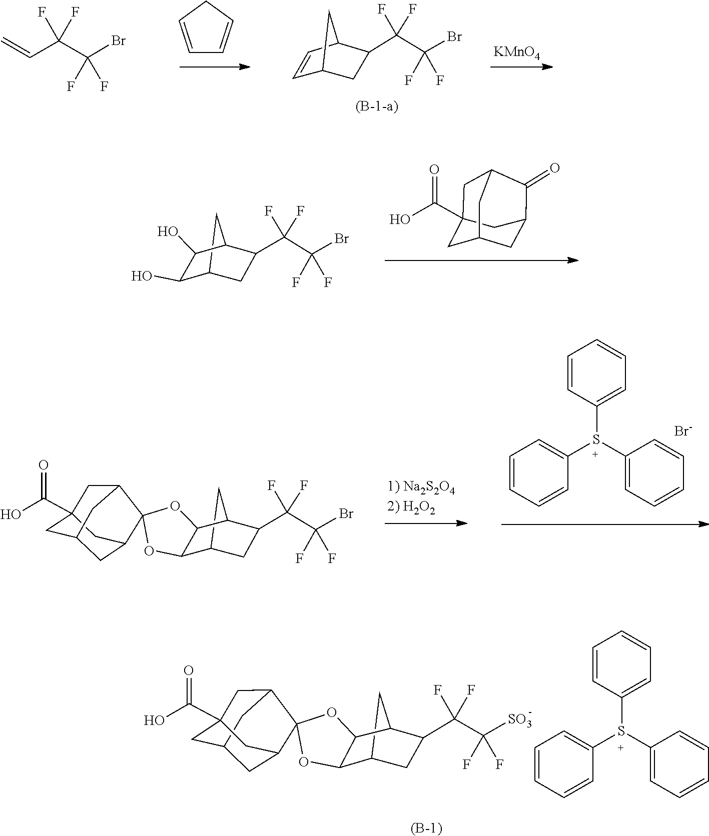

Into 20.0 mmol of 4-bromo-3,3,4,4-tetrafluoro-1-butene in a reaction container, 20.0 mmol of cyclopentadiene and 50 g of methylene chloride were added, and the mixture was stirred at room temperature for 3 hours. Thereafter, water was added to dilute the mixture, methylene chloride was added to extract the product, and the organic layer was separated. The obtained organic layer was washed with a saturated sodium chloride aqueous solution and then water. After drying with sodium sulfate, the solvent was distilled off, and the product was purified by column chromatography to obtain an olefin product (hereinafter, also referred to as “olefin product (B-1-a)”) in a good yield.

Into the olefin product (B-1-a), 40.0 mmol of potassium permanganate and 50 g of acetonitrile were added, and the mixture was stirred at 50° C. for 10 hours. Thereafter, a saturated sodium thiosulfate aqueous solution was added to terminate the reaction, then ethyl acetate was added to extract the product, and the organic layer was separated. The obtained organic layer was washed with a saturated sodium chloride aqueous solution, and then washed with water. After drying with sodium sulfate, the solvent was distilled off, and the product was purified by column chromatography to obtain a diol product in a good yield.

Into the diol product, 20.0 mmol of 2-adamantanone-5-carboxylic acid, 2.00 mmol of sulfuric acid, and 50 g of dichloromethane were added, and the mixture was stirred at room temperature for 24 hours. Thereafter, water was added to dilute the mixture, ethyl acetate was added to extract the product, and the organic layer was separated. The obtained organic layer was washed with a saturated sodium chloride aqueous solution, and then washed with water. After drying with sodium sulfate, the solvent was distilled off, and the product was purified by column chromatography to obtain an acetal product in a good yield.

Into the acetal product, a mixed liquid of acetonitrile water (1:1 (mass ratio)) was added to prepare a 1-M solution, then 40.0 mmol of sodium dithionite and 60.0 mmol of sodium hydrogen carbonate were added, and a reaction was performed at 70° C. for 4 hours. The product was extracted with acetonitrile, the solvent was distilled off, and then a mixed liquid of acetonitrile:water (3:1 (mass ratio)) was added to prepare a 0.5-M solution. Into this solution, 60.0 mmol of aqueous hydrogen peroxide and 2.00 mmol of sodium tungstate were added, and the mixture was stirred with heating at 50° C. for 12 hours. The product was extracted with acetonitrile, and the solvent was distilled off to obtain a sodium sulfonate salt compound. Into the sodium sulfonate salt compound, 20.0 mmol of triphenylsulfonium bromide was added, and a mixed liquid of water:dichloromethane (1:3 (mass ratio)) was added to prepare a 0.5-M solution. The solution was vigorously stirred at room temperature for 3 hours, then dichloromethane was added to extract the product, and the organic layer was separated. The obtained organic layer was dried with sodium sulfate, then the solvent was distilled off, and the product was purified by column chromatography to obtain a compound (B-1) represented by the formula (B-1) in a good yield.

### Synthesis Example 25 and Synthesis Example 32

### (Synthesis of Compound (B-2) to Compound (B-9))

An onium salt compound represented by each of the following formulae (B-2) to (B-9) was synthesized in the same manner as in Synthesis Example 24 except that the raw material and the precursor were appropriately changed.

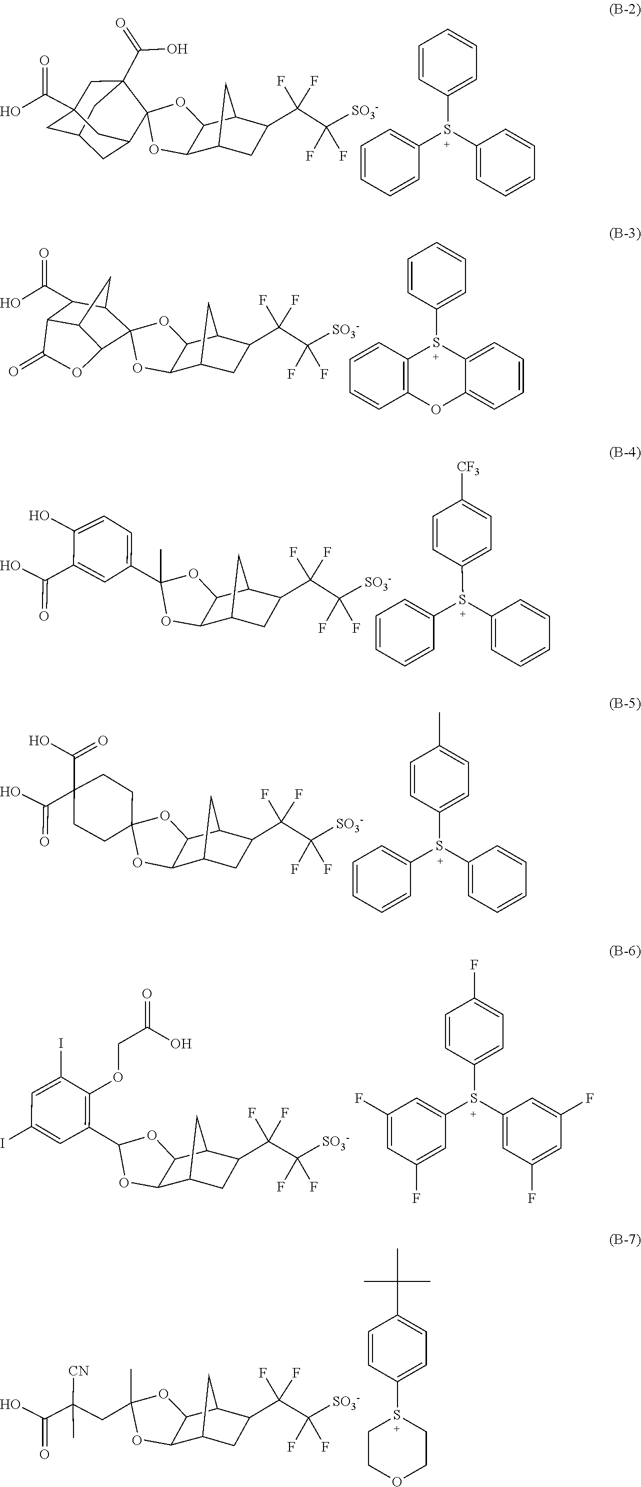

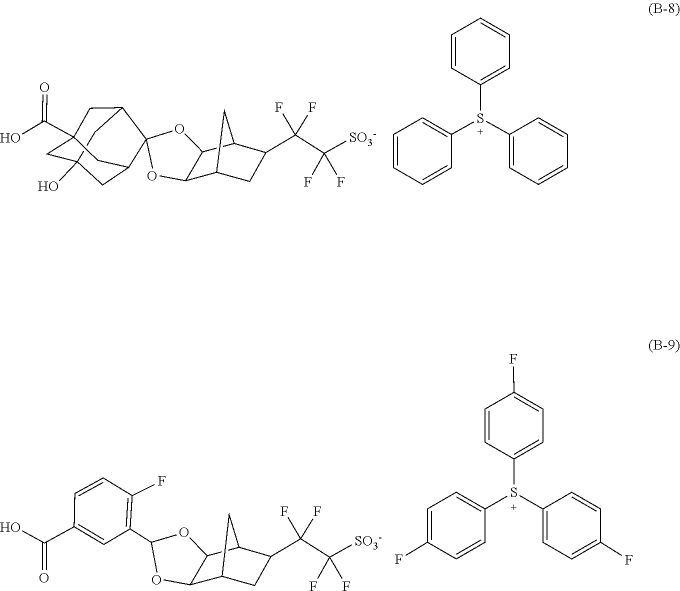

### Synthesis Example 33

### (Synthesis of Compound (B-10)

A compound (B-10) was synthesized according to the following synthesis scheme.

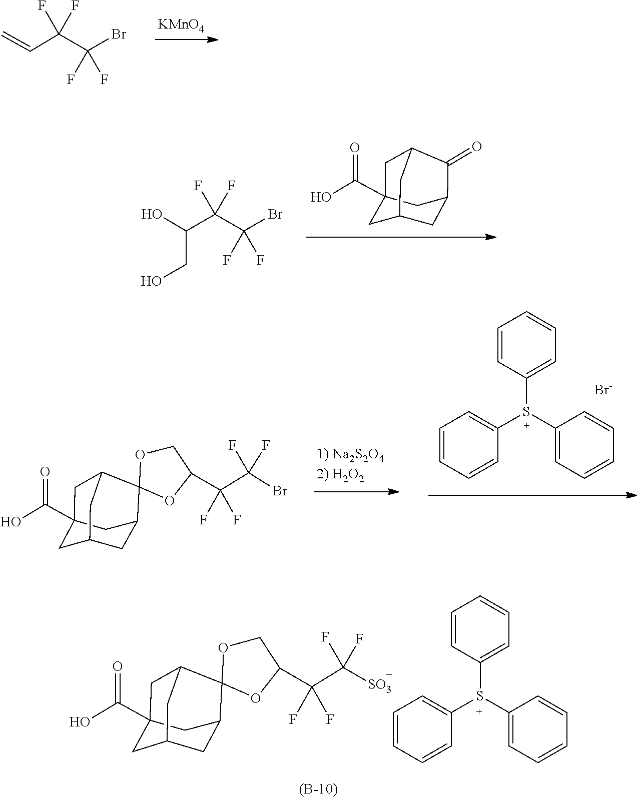

Into a reaction container, 20.0 mmol of 4-bromo-3,3,4,4-tetrafluoro-1-butene, 40.0 mmol of potassium permanganate, and 50 g of acetonitrile were added, and the mixture was stirred at 50° C. for 10 hours. Thereafter, a saturated sodium thiosulfate aqueous solution was added to terminate the reaction, then ethyl acetate was added to extract the product, and the organic layer was separated. The obtained organic layer was washed with a saturated sodium chloride aqueous solution, and then washed with water. After drying with sodium sulfate, the solvent was distilled off, and the product was purified by column chromatography to obtain a diol product in a good yield.

Into the diol product, 20.0 mmol of 2-adamantanone-5-carboxylic acid, 2.00 mmol of sulfuric acid, and 50 g of dichloromethane were added, and the mixture was stirred at room temperature for 24 hours. Thereafter, water was added to dilute the mixture, ethyl acetate was added to extract the product, and the organic layer was separated. The obtained organic layer was washed with a saturated sodium chloride aqueous solution, and then washed with water. After drying with sodium sulfate, the solvent was distilled off, and the product was purified by column chromatography to obtain an acetal product in a good yield.

Into the acetal product, a mixed liquid of acetonitrile water (1:1 (mass ratio)) was added to prepare a 1-M solution, then 40.0 mmol of sodium dithionite and 60.0 mmol of sodium hydrogen carbonate were added, and a reaction was performed at 70° C. for 4 hours. The product was extracted with acetonitrile, the solvent was distilled off, and then a mixed liquid of acetonitrile:water (3:1 (mass ratio)) was added to prepare a 0.5-M solution. Into this solution, 60.0 mmol of aqueous hydrogen peroxide and 2.00 mmol of sodium tungstate were added, and the mixture was stirred with heating at 50° C. for 12 hours. The product was extracted with acetonitrile, and the solvent was distilled off to obtain a sodium sulfonate salt compound. Into the sodium sulfonate salt compound, 20.0 mmol of triphenylsulfonium bromide was added, and a mixed liquid of water:dichloromethane (1:3 (mass ratio)) was added to prepare a 0.5-M solution. The solution was vigorously stirred at room temperature for 3 hours, then dichloromethane was added to extract the product, and the organic layer was separated. The obtained organic layer was dried with sodium sulfate, then the solvent was distilled off, and the product was purified by column chromatography to obtain a compound (B-10) represented by the formula (B-10) in a good yield.

### Synthesis Example 34 and Synthesis Example 35

### (Synthesis of Compound (B-11) and Compound (B-12))

An onium salt compound represented by each of the following formulae (B-11) and (B-12) was synthesized in the same manner as in Synthesis Example 33 except that the raw material and the precursor were appropriately changed.

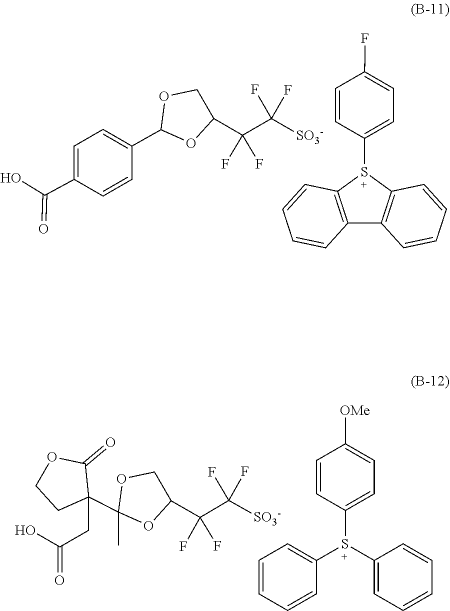

### Synthesis Example 36

### (Synthesis of Compound (B-13)

A compound (B-13) was synthesized according to the following synthesis scheme.

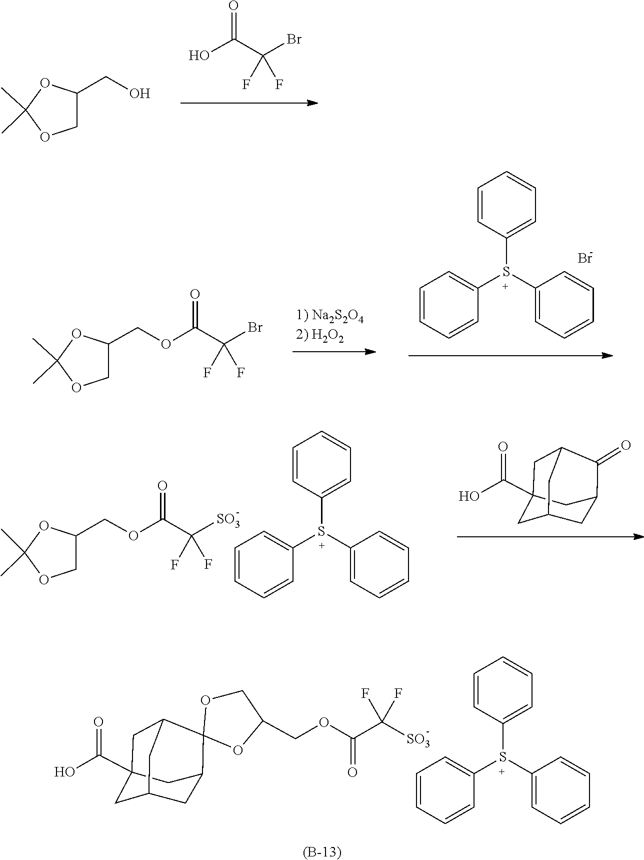

Into a reaction container, 20.0 mmol of 1,2-isopropylidene glycol, 20.0 mmol of bromodifluoroacetic acid, 30.0 mmol of dicyclohexylcarbodiimide, and 50 g of acetonitrile were added, and the mixture was stirred at room temperature for 4 hours. Thereafter, water was added to dilute the mixture, ethyl acetate was added to extract the product, and the organic layer was separated. The obtained organic layer was washed with a saturated sodium chloride aqueous solution, and then washed with water. After drying with sodium sulfate, the solvent was distilled off, and the product was purified by column chromatography to obtain an ester product in a good yield.

Into the ester product, a mixed liquid of acetonitrile water (1:1 (mass ratio)) was added to prepare a 1-M solution, then 40.0 mmol of sodium dithionite and 60.0 mmol of sodium hydrogen carbonate were added, and a reaction was performed at 70° C. for 4 hours. The product was extracted with acetonitrile, the solvent was distilled off, and then a mixed liquid of acetonitrile:water (3:1 (mass ratio)) was added to prepare a 0.5-M solution. Into this solution, 60.0 mmol of aqueous hydrogen peroxide and 2.00 mmol of sodium tungstate were added, and the mixture was stirred with heating at 50° C. for 12 hours. The product was extracted with acetonitrile, and the solvent was distilled off to obtain a sodium sulfonate salt compound. Into the sodium sulfonate salt compound, 20.0 mmol of triphenylsulfonium bromide was added, and a mixed liquid of water:dichloromethane (1:3 (mass ratio)) was added to prepare a 0.5-M solution. The solution was vigorously stirred at room temperature for 3 hours, then dichloromethane was added to extract the product, and the organic layer was separated. The obtained organic layer was dried with sodium sulfate, then the solvent was distilled off, and the product was purified by column chromatography to obtain an onium salt product in a good yield.

Into the onium salt product, 20.0 mmol of 2-adamantanone-5-carboxylic acid, 2.00 mmol of sulfuric acid, and 50 g of dichloroethane were added, and the mixture was stirred at 70° C. for 24 hours. Thereafter, water was added to dilute the mixture, then dichloromethane was added to extract the product, and the organic layer was separated. The obtained organic layer was washed with a saturated sodium chloride aqueous solution and then water. After drying with sodium sulfate, the solvent was distilled off, and the product was purified by column chromatography to obtain a compound (B-13) represented by the formula (B-13) in a good yield.

### Synthesis Example 37 to Synthesis Example 40

### (Synthesis of Compound (B-14) to Compound (B-17))

An onium salt compound represented by each of the following formulae (B-14) to (B-17) was synthesized in the same manner as in Synthesis Example 36 except that the raw material and the precursor were appropriately changed.

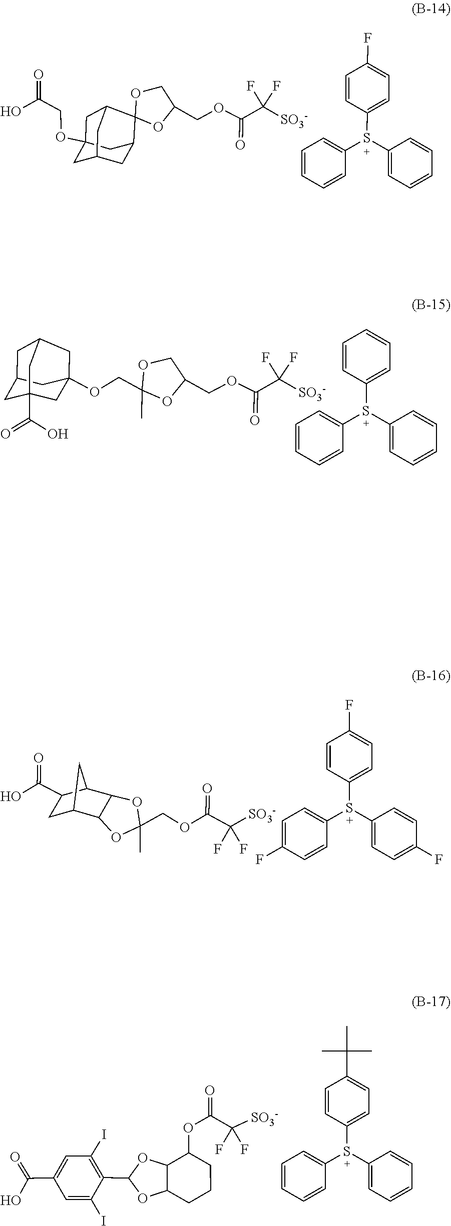

### Synthesis Example 41

### (Synthesis of Compound (B-18))

A compound (B-18) was synthesized according to the following synthesis scheme.

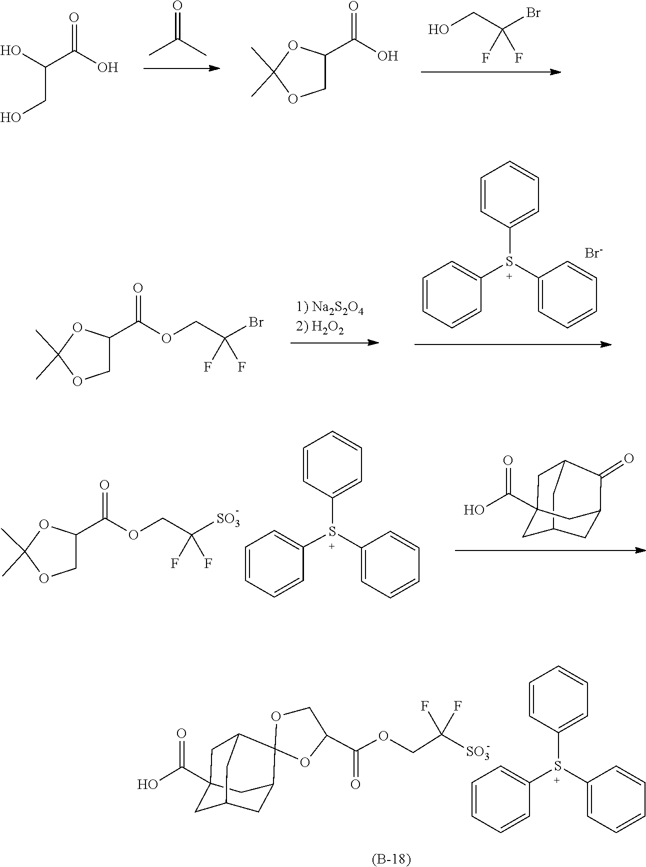

Into a reaction container, 20.0 mmol of glyceric acid, 2.00 mmol of sulfuric acid, and 50 g of acetone were added, and the mixture was stirred at room temperature for 2 hours. Thereafter, water was added to dilute the mixture, ethyl acetate was added to extract the product, and the organic layer was separated. The obtained organic layer was washed with a saturated sodium chloride aqueous solution and then water. After drying with sodium sulfate, the solvent was distilled off, and the product was purified by column chromatography to obtain an acetal product in a good yield.

Into the acetal product, 20.0 mmol of 2-bromo-2,2-difluoroethane-2-ol, 30.0 mmol of dicyclohexylcarbodiimide, and 50 g of dichloromethane were added, and the mixture was stirred at room temperature for 4 hours. Thereafter, water was added to dilute the mixture, dichloromethane was added to extract the product, and the organic layer was separated. The obtained organic layer was washed with a saturated sodium chloride aqueous solution and then water. After drying with sodium sulfate, the solvent was distilled off, and the product was purified by column chromatography to obtain an ester product in a good yield.

Into the ester product, a mixed liquid of acetonitrile water (1:1 (mass ratio)) was added to prepare a 1-M solution, then 40.0 mmol of sodium dithionite and 60.0 mmol of sodium hydrogen carbonate were added, and a reaction was performed at 70° C. for 4 hours. The product was extracted with acetonitrile, the solvent was distilled off, and then a mixed liquid of acetonitrile:water (3:1 (mass ratio)) was added to prepare a 0.5-M solution. Into this solution, 60.0 mmol of aqueous hydrogen peroxide and 2.00 mmol of sodium tungstate were added, and the mixture was stirred with heating at 50° C. for 12 hours. The product was extracted with acetonitrile, and the solvent was distilled off to obtain a sodium sulfonate salt compound. Into the sodium sulfonate salt compound, 20.0 mmol of triphenylsulfonium bromide was added, and a mixed liquid of water:dichloromethane (1:3 (mass ratio)) was added to prepare a 0.5-M solution. The solution was vigorously stirred at room temperature for 3 hours, then dichloromethane was added to extract the product, and the organic layer was separated. The obtained organic layer was dried with sodium sulfate, then the solvent was distilled off, and the product was purified by column chromatography to obtain an onium salt product in a good yield.

Into the onium salt product, 20.0 mmol of 2-adamantanone-5-carboxylic acid, 2.00 mmol of sulfuric acid, and 50 g of dichloroethane were added, and the mixture was stirred at 70° C. for 20 hours. Thereafter, water was added to dilute the mixture, dichloromethane was added to extract the product, and the organic layer was separated. The obtained organic layer was washed with a saturated sodium chloride aqueous solution and then water. After drying with sodium sulfate, the solvent was distilled off, and the product was purified by column chromatography to obtain a compound (B-18) represented by the formula (B-18) in a good yield.

### Synthesis Example 42 to Synthesis Example 45

### (Synthesis of Compound (B-19) to Compound (B-22))

Onium salt compounds represented by the following formula (B-19) to (B-22) were synthesized in the same manner as in Synthesis Example 41 except that the raw material and the precursor were appropriately changed.

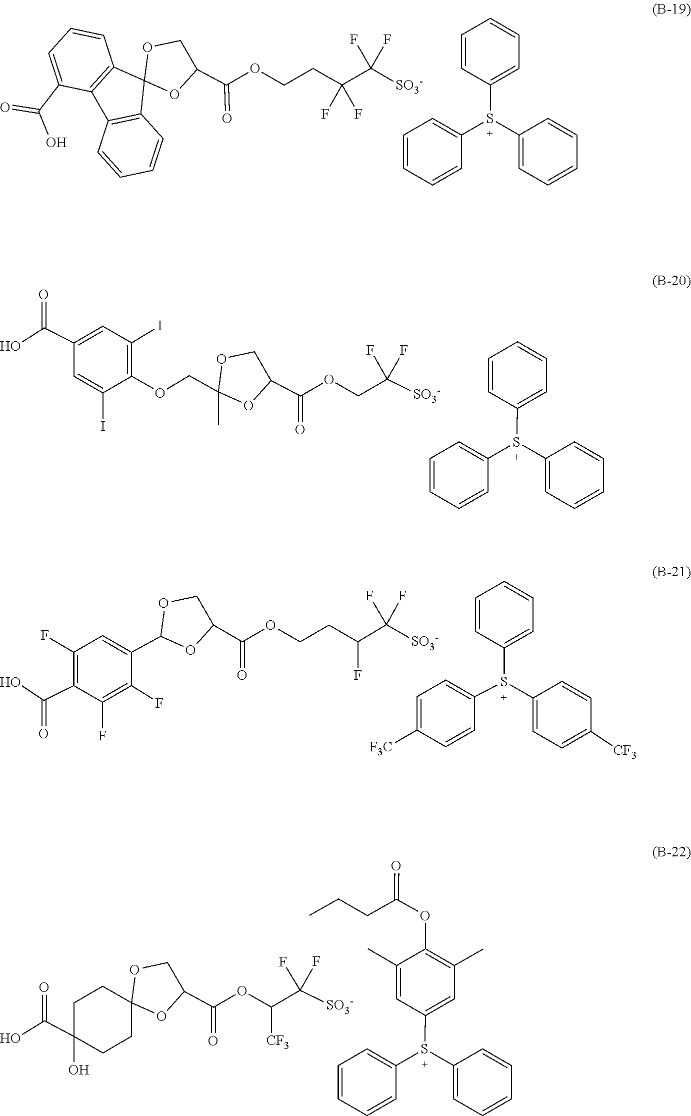

### Synthesis Example 46

### (Synthesis of Compound (B-23))

A compound (B-23) was synthesized according to the following synthesis scheme.

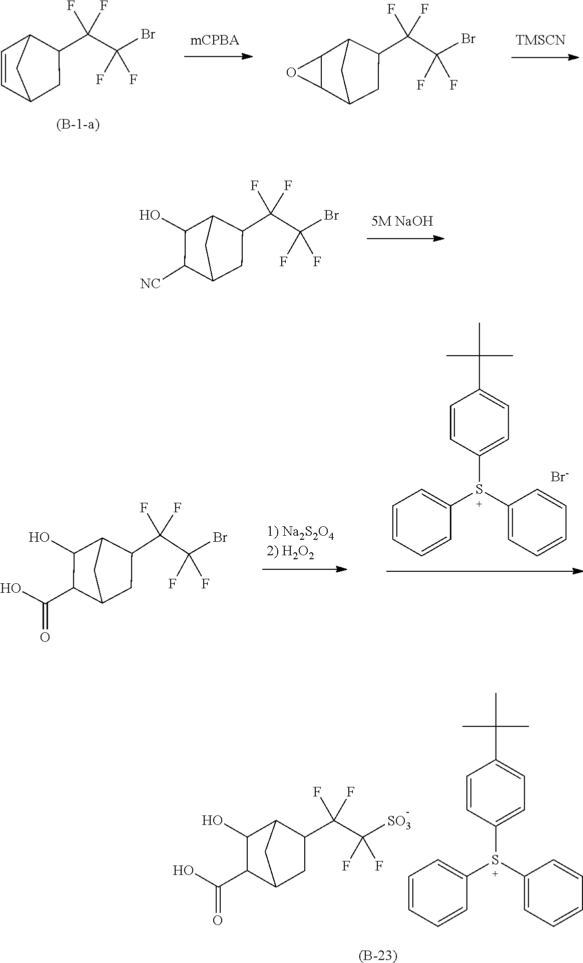

Into a reaction container, 20.0 mmol of the olefin product (B-1-a), 25.0 mmol of meta-chloroperoxybenzoic acid, and 50 g of dichloromethane were added, and the mixture was stirred at room temperature for 4 hours. Thereafter, a saturated sodium sulfite aqueous solution was added to terminate the reaction, then ethyl acetate was added to extract the product, and the organic layer was separated. The obtained organic layer was washed with a saturated sodium hydrogen carbonate aqueous solution and then water. After drying with sodium sulfate, the solvent was distilled off, and the product was purified by column chromatography to obtain an epoxy product in a good yield.

Into the epoxy product, 20.0 mmol of trimethylsilyl cyanide, 1.00 mmol of N-methylmorpholine-N-oxide, and 50 g of dichloromethane were added, and the mixture was stirred at room temperature for 4 hours. Thereafter, 2-M hydrochloric acid was added to terminate the reaction, then dichloromethane was added to extract the product, and the organic layer was separated. The obtained organic layer was washed with a saturated sodium hydrogen carbonate aqueous solution and then water. After drying with sodium sulfate, the solvent was distilled off, and the product was purified by column chromatography to obtain a cyano product in a good yield.

Into the cyano product, 50 g of a 5-M sodium hydroxide aqueous solution was added, and the mixture was stirred at 100° C. for 12 hours. Thereafter, 1-M hydrochloric acid was added to terminate the reaction, then ethyl acetate was added to extract the product, and the organic layer was separated. The obtained organic layer was dried with sodium sulfate, the solvent was distilled off, and the product was purified by column chromatography to obtain a carboxylic acid product in a good yield.

Into the carboxylic acid product, a mixed liquid of acetonitrile:water (1:1 (mass ratio)) was added to prepare a 1-M solution, then 40.0 mmol of sodium dithionite and 60.0 mmol of sodium hydrogen carbonate were added, and a reaction was performed at 70° C. for 4 hours. The product was extracted with acetonitrile, the solvent was distilled off, and then a mixed liquid of acetonitrile:water (3:1 (mass ratio)) was added to prepare a 0.5-M solution. Into this solution, 60.0 mmol of aqueous hydrogen peroxide and 2.00 mmol of sodium tungstate were added, and the mixture was stirred with heating at 50° C. for 12 hours. The product was extracted with acetonitrile, and the solvent was distilled off to obtain a sodium sulfonate salt compound. Into the sodium sulfonate salt compound, 20.0 mmol of (4-(tert-butyl)phenyl)diphenylsulfonium bromide was added, and a mixed liquid of water:dichloromethane (1:3 (mass ratio)) was added to prepare a 0.5-M solution. The solution was vigorously stirred at room temperature for 3 hours, then dichloromethane was added to extract the product, and the organic layer was separated. The obtained organic layer was dried with sodium sulfate, then the solvent was distilled off, and the product was purified by column chromatography to obtain a compound (B-23) represented by the formula (B-23) in a good yield.

### Synthesis Example 471

### (Synthesis of Compound (B-24))

A compound (B-24) was synthesized according to the following synthesis scheme.

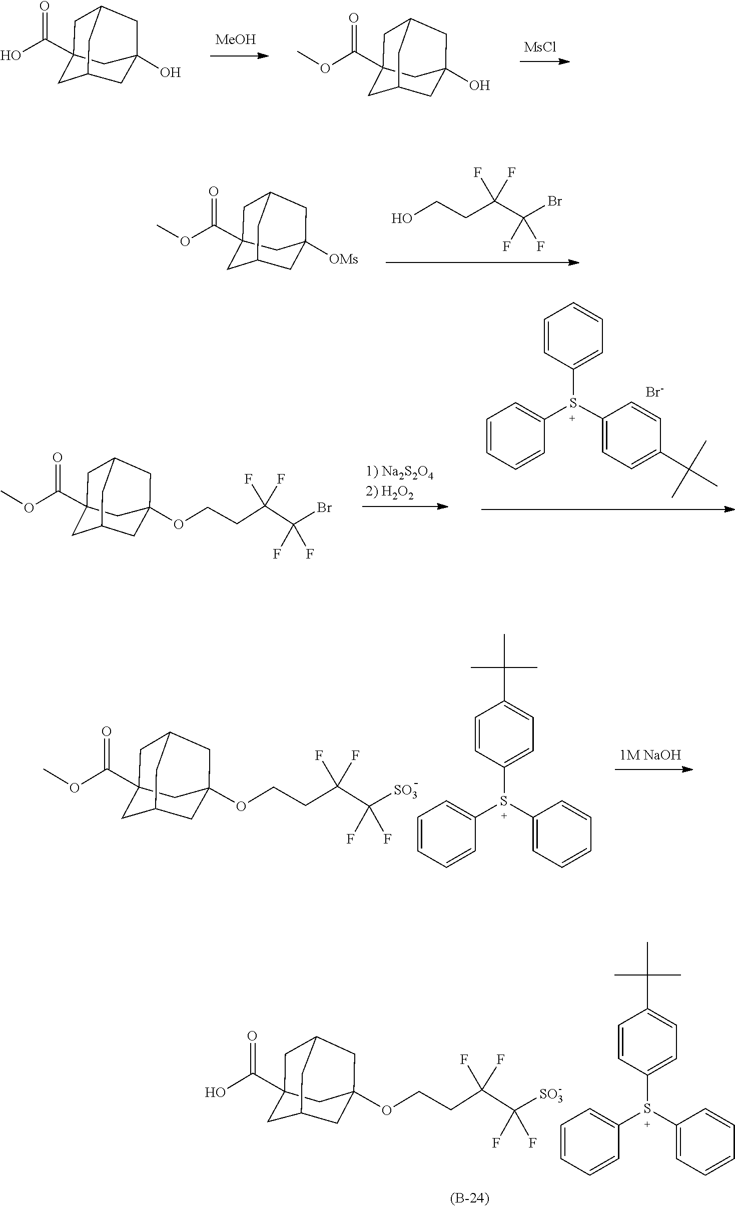

Into a reaction container, 20.0 mmol of 3-hydroxy-1-adamantanecarboxylic acid, 20.0 mmol of methanol, 30.0 mmol of dicyclohexylcarbodiimide, and 50 g of dichloromethane were added, and the mixture was stirred at room temperature for 4 hours. Thereafter, water was added to dilute the mixture, dichloromethane was added to extract the product, and the organic layer was separated. The obtained organic layer was washed with a saturated sodium chloride aqueous solution and then water. After drying with sodium sulfate, the solvent was distilled off, and the product was purified by column chromatography to obtain an ester product in a good yield.

Into the ester product, 30.0 mmol of mesyl chloride, 30.0 mmol of triethylamine, and 50 g of dichloromethane were added, and the mixture was stirred at room temperature for 3 hours. Thereafter, a saturated ammonium chloride aqueous solution was added to terminate the reaction, then dichloromethane was added to extract the product, and the organic layer was separated. The obtained organic layer was washed with a saturated sodium chloride aqueous solution and then water. After drying with sodium sulfate, the solvent was distilled off, and the product was purified by column chromatography to obtain a mesyl product in a good yield.

Into the mesyl product, 30.0 mmol of 4-bromo-3,3,4,4-tetrafluoro-1-ol, 30.0 mmol of diazabicycloundecene, and 50 g of acetonitrile were added at 80° C. for 24 hours. Thereafter, a saturated ammonium chloride aqueous solution was added to terminate the reaction, then ethyl acetate was added to extract the product, and the organic layer was separated. The obtained organic layer was washed with a saturated sodium chloride aqueous solution and then water. After drying with sodium sulfate, the solvent was distilled off, and the product was purified by column chromatography to obtain an alkoxy product in a good yield.

Into the alkoxy product, a mixed liquid of acetonitrile water (1:1 (mass ratio)) was added to prepare a 1-M solution, then 40.0 mmol of sodium dithionite and 60.0 mmol of sodium hydrogen carbonate were added, and a reaction was performed at 70° C. for 4 hours. The product was extracted with acetonitrile, the solvent was distilled off, and then a mixed liquid of acetonitrile:water (3:1 (mass ratio)) was added to prepare a 0.5-M solution. Into this solution, 60.0 mmol of aqueous hydrogen peroxide and 2.00 mmol of sodium tungstate were added, and the mixture was stirred with heating at 50° C. for 12 hours. The product was extracted with acetonitrile, and the solvent was distilled off to obtain a sodium sulfonate salt compound. Into the sodium sulfonate salt compound, 20.0 mmol of (4-(tert-butyl)phenyl)diphenylsulfonium bromide was added, and a mixed liquid of water:dichloromethane (1:3 (mass ratio)) was added to prepare a 0.5-M solution. The solution was vigorously stirred at room temperature for 3 hours, then dichloromethane was added to extract the product, and the organic layer was separated. The obtained organic layer was dried with sodium sulfate, then the solvent was distilled off, and the product was purified by column chromatography to obtain an onium salt product in a good yield.

Into the onium salt product, 50 g of 1-M sodium hydroxide aqueous solution and 50 g of methanol were added, and the mixture was stirred at 100° C. for 5 hours. Thereafter, 1-M hydrochloric acid was added to terminate the reaction, then dichloromethane was added to extract the product, and the organic layer was separated. The obtained organic layer was dried with sodium sulfate, the solvent was distilled off, and the product was purified by column chromatography to obtain a compound (B-24) represented by the formula (B-24) in a good yield.

<Onium Salts other than Compound (B-1) to Compound (B-24)>
bb-1 to bb-7: Compounds represented by the following formula (bb-1) to formula (bb-7) (hereinafter, the compounds represented by the formula (bb-1) to the formula (bb-7) may be respectively described as “compound (bb-1)” to “compound (bb-7)”).

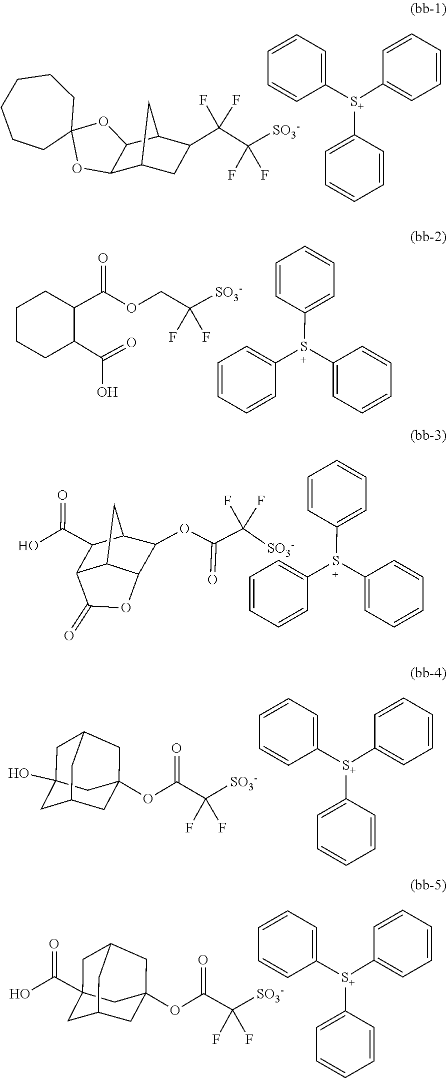

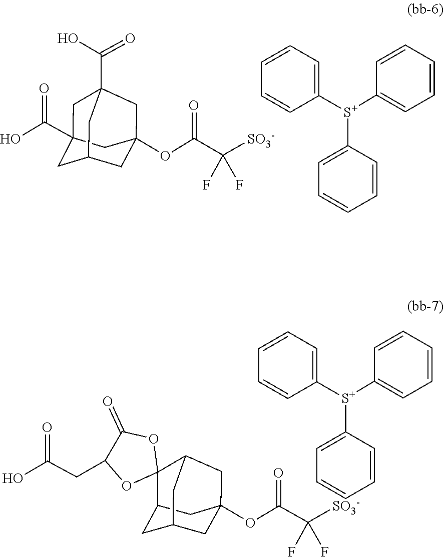

### <[C] Acid-Diffusion Inhibitor>

C-1 to C-8: Compounds represented by the following formula (C-1) to formula (C-8) (hereinafter, the compounds represented by the formula (C-1) to the formula (C-8) may be respectively described as “compound (C-1)” to “compound (C-8)”).

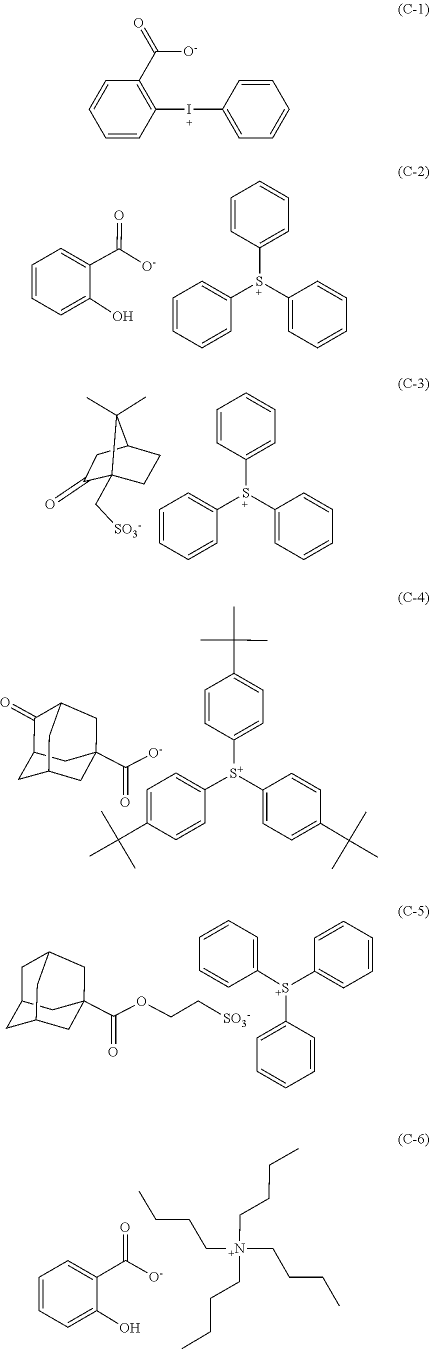

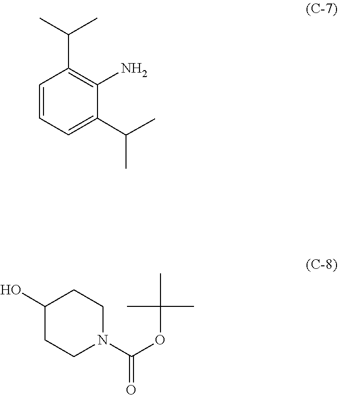

### <[E] Solvent>

E-1: Propylene glycol monomethyl ether acetate
E-2: Propylene glycol monomethyl ether
E-3: γ-Butyrolactone
E-4: Ethyl lactate

### <Preparation of Positive-Type Radiation-Sensitive Resin Composition for ArF Liquid-Immersion Exposure>

### Example 1

Mixing 100 parts by mass of (A-1) as the resin [A], 10.0 parts by mass of (B-1) as the radiation-sensitive acid generator [B], 8.0 parts by mass of (C-1) as the acid-diffusion inhibitor [C], 3.0 parts by mass (solid content) of (F-1) as the high-fluorine-content resin [F], and 3,230 parts by mass of a mixed solvent of (E-1)/(E-2)/(E-3) (2,240/960/30 (parts by mass)) as the solvent [E] was performed, and the mixture was filtered with a membrane filter having a pore diameter of 0.2 μm to prepare a radiation-sensitive resin composition (J-1).

### Examples 2 to 51 and Comparative Examples 1 to 7

Radiation-sensitive resin compositions (J-2) to (J-51) and (CJ-1) to (CJ-7) were prepared in the same manner as in Example 1 except that components with types and contents described in the following Table 4 and Table 5 were used.

TABLE 4

| | | Example 1 | Example 2 | Example 3 | Example 4 | Example 5 | Example 6 |
|---|---|---|---|---|---|---|---|
| Radiation-sensitive resin | | J-1 | J-2 | J-3 | J-4 | J-5 | J-6 |
| composition | | | | | | | |
| [A] Resin | Type | A-1 | A-2 | A-3 | A-4 | A-5 | A-6 |
| | Amount | 100 | 100 | 100 | 100 | 100 | 100 |
| | (part(s) | | | | | | |
| | by mass) | | | | | | |
| [B] | Type | B-1 | B-1 | B-1 | B-1 | B-1 | B-1 |
| Radiation- | Amount | 10.0 | 10.0 | 10.0 | 10.0 | 10.0 | 10.0 |
| sensitive | (part(s) | | | | | | |
| Acid | by mass) | | | | | | |
| Generater | | | | | | | |
| [C] Acid- | Type | C-1 | C-1 | C-1 | C-1 | C-1 | C-1 |
| diffusion | Amount | 8.0 | 8.0 | 8.0 | 8.0 | 8.0 | 8.0 |
| Inhibitor | (part(s) | | | | | | |
| | by mass) | | | | | | |
| [F] High- | Type | F-1 | F-1 | F-1 | F-1 | F-1 | F-1 |
| Fluorine- | Amount | 3.0 | 3.0 | 3.0 | 3.0 | 3.0 | 3.0 |
| Content- | (part(s) | | | | | | |
| Resin | by mass) | | | | | | |
| [E] Solvent | Type | E-1/E-2/ | E-1/E-2/ | E-1/E-2/ | E-1/E-2/ | E-1/E-2/ | E-1/E-2/ |
| | | E-3 | E-3 | E-3 | E-3 | E-3 | E-3 |
| | Amount | 2240/960/30 | 2240/960/30 | 2240/960/30 | 2240/960/30 | 2240/960/30 | 2240/960/30 |
| | (part(s) | | | | | | |
| | by mass) | | | | | | |
| | | | | | Example | Example | Example |
| | | Example 7 | Example 8 | Example 9 | 10 | 11 | 12 |
| Radiation-sensitive resin | | J-7 | J-8 | J-9 | J-10 | J-11 | J-12 |
| composition | | | | | | | |
| [A] Resin | Type | A-7 | A-8 | A-9 | A-10 | A-11 | A-12 |
| | Amount | 100 | 100 | 100 | 100 | 100 | 100 |
| | (part(s) | | | | | | |
| | by mass) | | | | | | |
| [B] | Type | B-1 | B-1 | B-1 | B-1 | B-1 | B-1 |
| Radiation- | Amount | 10.0 | 10.0 | 10.0 | 10.0 | 10.0 | 10.0 |
| sensitive | (part(s) | | | | | | |
| Acid | by mass) | | | | | | |
| Generater | | | | | | | |
| [C] Acid- | Type | C-1 | C-1 | C-1 | C-1 | C-1 | C-1 |
| diffusion | Amount | 8.0 | 8.0 | 8.0 | 8.0 | 8.0 | 8.0 |
| Inhibitor | (part(s) | | | | | | |
| | by mass) | | | | | | |
| [F] High- | Type | F-1 | F-1 | F-1 | F-1 | F-1 | F-1 |
| Fluorine- | Amount | 3.0 | 3.0 | 3.0 | 3.0 | 3.0 | 3.0 |
| Content- | (part(s) | | | | | | |
| Resin | by mass) | | | | | | |
| [E] Solvent | Type | E-1/E-2/ | E-1/E-2/ | E-1/E-2/ | E-1/E-2/ | E-1/E-2/ | E-1/E-2/ |
| | | E-3 | E-3 | E-3 | E-3 | E-3 | E-3 |
| | Amount | 2240/960/30 | 2240/960/30 | 2240/960/30 | 2240/960/30 | 2240/960/30 | 2240/960/30 |
| | (part(s) | | | | | | |
| | by mass) | | | | | | |
| | | Example | Example | Example | Example | Example | Example |
| | | 13 | 14 | 15 | 16 | 17 | 18 |
| Radiation-sensitive resin | | J-13 | J-14 | J-15 | J-16 | J-17 | J-18 |
| composition | | | | | | | |
| [A] Resin | Type | A-13 | A-1 | A-1 | A-1 | A-1 | A-1 |
| | Amount | 100 | 100 | 100 | 100 | 100 | 100 |
| | (part(s) | | | | | | |
| | by mass) | | | | | | |
| [B] | Type | B-1 | B-2 | B-3 | B-4 | B-5 | B-6 |
| Radiation- | Amount | 10.0 | 10.0 | 10.0 | 10.0 | 10.0 | 10.0 |
| sensitive | (part(s) | | | | | | |
| Acid | by mass) | | | | | | |
| Generater | | | | | | | |
| [C] Acid- | Type | C-1 | C-1 | C-1 | C-1 | C-1 | C-1 |
| diffusion | Amount | 8.0 | 8.0 | 8.0 | 8.0 | 8.0 | 8.0 |
| Inhibitor | (part(s) | | | | | | |
| | by mass) | | | | | | |
| [F] High- | Type | F-1 | F-1 | F-1 | F-1 | F-1 | F-1 |
| Fluorine- | Amount | 3.0 | 3.0 | 3.0 | 3.0 | 3.0 | 3.0 |
| Content- | (part(s) | | | | | | |
| Resin | by mass) | | | | | | |
| [E] Solvent | Type | E-1/E-2/ | E-1/E-2/ | E-1/E-2/ | E-1/E-2/ | E-1/E-2/ | E-1/E-2/ |
| | | E-3 | E-3 | E-3 | E-3 | E-3 | E-3 |
| | Amount | 2240/960/30 | 2240/960/30 | 2240/960/30 | 2240/960/30 | 2240/960/30 | 2240/960/30 |
| | (part(s) | | | | | | |
| | by mass) | | | | | | |
| | | Example | Example | Example | Example | Example | Example |
| | | 19 | 20 | 21 | 22 | 23 | 24 |
| Radiation-sensitive resin | | J-19 | J-20 | J-21 | J-22 | J-23 | J-24 |
| composition | | | | | | | |
| [A] Resin | Type | A-1 | A-1 | A-1 | A-1 | A-1 | A-1 |
| | Amount | 100 | 100 | 100 | 100 | 100 | 100 |
| | (part(s) | | | | | | |
| | by mass) | | | | | | |
| [B] | Type | B-7 | B-8 | B-9 | B-10 | B-11 | B-12 |
| Radiation- | Amount | 10.0 | 10.0 | 10.0 | 10.0 | 10.0 | 10.0 |
| sensitive | (part(s) | | | | | | |
| Acid | by mass) | | | | | | |
| Generater | | | | | | | |
| [C] Acid- | Type | C-1 | C-1 | C-1 | C-1 | C-1 | C-1 |
| diffusion | Amount | 8.0 | 8.0 | 8.0 | 8.0 | 8.0 | 8.0 |
| Inhibitor | (part(s) | | | | | | |
| | by mass) | | | | | | |
| [F] High- | Type | F-1 | F-1 | F-1 | F-1 | F-1 | F-1 |
| Fluorine- | Amount | 3.0 | 3.0 | 3.0 | 3.0 | 3.0 | 3.0 |
| Content- | (part(s) | | | | | | |
| Resin | by mass) | | | | | | |
| [E] Solvent | Type | E-1/E-2/ | E-1/E-2/ | E-1/E-2/ | E-1/E-2/ | E-1/E-2/ | E-1/E-2/ |
| | | E-3 | E-3 | E-3 | E-3 | E-3 | E-3 |
| | Amount | 2240/960/30 | 2240/960/30 | 2240/960/30 | 2240/960/30 | 2240/960/30 | 2240/960/30 |
| | (part(s) | | | | | | |
| | by mass) | | | | | | |

| | | Example 25 | Example 26 | Example 27 | Example 28 | Example 29 |
|---|---|---|---|---|---|---|
| Radiation-sensitive resin | | J-25 | J-26 | J-27 | J-28 | J-29 |
| composition | | | | | | |
| [A] Resin | Type | A-1 | A-1 | A-1 | A-1 | A-1 |
| | Amount | 100 | 100 | 100 | 100 | 100 |
| | (part(s) by | | | | | |
| | mass) | | | | | |
| [B] | Type | B-13 | B-14 | B-15 | B-16 | B-17 |
| Radiation- | Amount | 10.0 | 10.0 | 10.0 | 10.0 | 10.0 |
| sensitive | (part(s) by | | | | | |
| Acid | mass) | | | | | |
| Generater | | | | | | |
| [C] Acid- | Type | C-1 | C-1 | C-1 | C-1 | C-1 |
| diffusion | Amount | 8.0 | 8.0 | 8.0 | 8.0 | 8.0 |
| Inhibitor | (part(s) by | | | | | |
| | mass) | | | | | |
| [F] High- | Type | F-1 | F-1 | F-1 | F-1 | F-1 |
| Fluorine- | Amount | 3.0 | 3.0 | 3.0 | 3.0 | 3.0 |
| Content- | (part(s) by | | | | | |
| Resin | mass) | | | | | |
| [E] Solvent | Type | E-1/E-2/E-3 | E-1/E-2/E-3 | E-1/E-2/E-3 | E-1/E-2/E-3 | E-1/E-2/E-3 |
| | Amount | 2240/960/30 | 2240/960/30 | 2240/960/30 | 2240/960/30 | 2240/960/30 |
| | (part(s) by | | | | | |
| | mass) | | | | | |

TABLE 5

| | | Example | Example | Example | Example | Example | Example |
|---|---|---|---|---|---|---|---|
| | | 30 | 31 | 32 | 33 | 34 | 35 |
| Radiation-sensitive resin | | J-30 | J-31 | J-32 | J-33 | J-34 | J-35 |
| composition | | | | | | | |
| [A] Resin | Type | A-1 | A-1 | A-1 | A-1 | A-1 | A-1 |
| | Amount | 100 | 100 | 100 | 100 | 100 | 100 |
| | (part(s) | | | | | | |
| | by mass) | | | | | | |
| [B] | Type | B-18 | B-19 | B-20 | B-21 | B-22 | B-23 |
| Radiation- | Amount | 10.0 | 10.0 | 10.0 | 10.0 | 10.0 | 10.0 |
| sensitive | (part(s) | | | | | | |
| Acid | by mass) | | | | | | |
| Generater | | | | | | | |
| [C] Acid- | Type | C-1 | C-1 | C-1 | C-1 | C-1 | C-1 |
| diffusion | Amount | 8.0 | 8.0 | 8.0 | 8.0 | 8.0 | 8.0 |
| Inhibitor | (part(s) | | | | | | |
| | by mass) | | | | | | |
| [F] High- | Type | F-1 | F-1 | F-1 | F-1 | F-1 | F-1 |
| Fluorine- | Amount | 3.0 | 3.0 | 3.0 | 3.0 | 3.0 | 3.0 |
| Content- | (part(s) | | | | | | |
| Resin | by mass) | | | | | | |
| [E] Solvent | Type | E-1/E-2/ | E-1/E-2/ | E-1/E-2/ | E-1/E-2/ | E-1/E-2/ | E-1/E-2/ |
| | | E-3 | E-3 | E-3 | E-3 | E-3 | E-3 |
| | Amount | 2240/960/30 | 2240/960/30 | 2240/960/30 | 2240/960/30 | 2240/960/30 | 2240/960/30 |
| | (part(s) | | | | | | |
| | by mass) | | | | | | |
| | | Example | Example | Example | Example | Example | Example |
| | | 36 | 37 | 38 | 39 | 40 | 41 |
| Radiation-sensitive resin | | J-36 | J-37 | J-38 | J-39 | J-40 | J-41 |
| composition | | | | | | | |
| [A] Resin | Type | A-1 | A-1 | A-1 | A-1 | A-1 | A-1 |
| | Amount | 100 | 100 | 100 | 100 | 100 | 100 |
| | (part(s) | | | | | | |
| | by mass) | | | | | | |
| [B] | Type | B-24 | B-1 | B-1 | B-1 | B-1 | B-1 |
| Radiation- | Amount | 10.0 | 10.0 | 10.0 | 10.0 | 10.0 | 10.0 |
| sensitive | (part(s) | | | | | | |
| Acid | by mass) | | | | | | |
| Generater | | | | | | | |
| [C] Acid- | Type | C-1 | C-2 | C-3 | C-4 | C-5 | C-6 |
| diffusion | Amount | 8.0 | 8.0 | 8.0 | 8.0 | 8.0 | 8.0 |
| Inhibitor | (part(s) | | | | | | |
| | by mass) | | | | | | |
| [F] High- | Type | F-1 | F-1 | F-1 | F-1 | F-1 | F-1 |
| Fluorine- | Amount | 3.0 | 3.0 | 3.0 | 3.0 | 3.0 | 3.0 |
| Content- | (part(s) | | | | | | |
| Resin | by mass) | | | | | | |
| [E] Solvent | Type | E-1/E-2/ | E-1/E-2/ | E-1/E-2/ | E-1/E-2/ | E-1/E-2/ | E-1/E-2/ |
| | | E-3 | E-3 | E-3 | E-3 | E-3 | E-3 |
| | Amount | 2240/960/30 | 2240/960/30 | 2240/960/30 | 2240/960/30 | 2240/960/30 | 2240/960/30 |
| | (part(s) | | | | | | |
| | by mass) | | | | | | |
| | | Example | Example | Example | Example | Example | Example |
| | | 42 | 43 | 44 | 45 | 46 | 47 |
| Radiation-sensitive resin | | J-42 | J-43 | J-44 | J-45 | J-46 | J-47 |
| composition | | | | | | | |
| [A] Resin | Type | A-1 | A-1 | A-1 | A-1 | A-1 | A-1 |
| | Amount | 100 | 100 | 100 | 100 | 100 | 100 |
| | (part(s) | | | | | | |
| | by mass) | | | | | | |
| [B] | Type | B-1 | B-1 | B-1 | B-1 | B-1/B-11 | B-1/B-14 |
| Radiation- | Amount | 10.0 | 10.0 | 5.0 | 20.0 | 5.0/5.0 | 5.0/5.0 |
| sensitive | (part(s) | | | | | | |
| Acid | by mass) | | | | | | |
| Generater | | | | | | | |
| [C] Acid- | Type | C-7 | C-8 | C-1 | C-1 | C-1 | C-1 |
| diffusion | Amount | 8.0 | 8.0 | 8.0 | 8.0 | 8.0 | 8.0 |
| Inhibitor | (part(s) | | | | | | |
| | by mass) | | | | | | |
| [F] High- | Type | F-1 | F-1 | F-1 | F-1 | F-1 | F-1 |
| Fluorine- | Amount | 3.0 | 3.0 | 3.0 | 3.0 | 3.0 | 3.0 |
| Content- | (part(s) | | | | | | |
| Resin | by mass) | | | | | | |
| [E] Solvent | Type | E-1/E-2/ | E-1/E-2/ | E-1/E-2/ | E-1/E-2/ | E-1/E-2/ | E-1/E-2/ |
| | | E-3 | E-3 | E-3 | E-3 | E-3 | E-3 |
| | Amount | 2240/960/30 | 2240/960/30 | 2240/960/30 | 2240/960/30 | 2240/960/30 | 2240/960/30 |
| | (part(s) | | | | | | |
| | by mass) | | | | | | |
| | | | Example | Example | Example | Comparative | Comparative |
| | | Example 48 | 49 | 50 | 51 | Example 1 | Example 2 |
| Radiation-sensitive resin | | J-48 | J-49 | J-50 | J-51 | CJ-1 | CJ-2 |
| composition | | | | | | | |
| [A] Resin | Type | A-1 | A-1 | A-1 | A-1 | A-1 | A-1 |
| | Amount | 100 | 100 | 100 | 100 | 100 | 100 |
| | (part(s) | | | | | | |
| | by mass) | | | | | | |
| [B] | Type | B-1/B-22 | B-1 | B-1 | B-1 | bb-1 | bb-2 |
| Radiation- | Amount | 5.0/5.0 | 10.0 | 10.0 | 10.0 | 10.0 | 10.0 |
| sensitive | (part(s) | | | | | | |
| Acid | by mass) | | | | | | |
| Generater | | | | | | | |
| [C] Acid- | Type | C-1 | C-1 | C-1 | C-1 | C-1 | C-1 |
| diffusion | Amount | 8.0 | 8.0 | 8.0 | 8.0 | 8.0 | 8.0 |
| Inhibitor | (part(s) | | | | | | |
| | by mass) | | | | | | |
| [F] High- | Type | F-1 | F-2 | F-3 | F-4 | F-1 | F-1 |
| Fluorine- | Amount | 3.0 | 3.0 | 3.0 | 3.0 | 3.0 | 3.0 |
| Content- | (part(s) | | | | | | |
| Resin | by mass) | | | | | | |
| [E] Solvent | Type | E-1/E-2/E-3 | E-1/E-2/E-3 | E-1/E-2/E-3 | E-1/E-2/E-3 | E-1/E-2/E-3 | E-1/E-2/E-3 |
| | Amount | 2240/960/30 | 2240/960/30 | 2240/960/30 | 2240/960/30 | 2240/960/30 | 2240/960/30 |
| | (part(s) | | | | | | |
| | by mass) | | | | | | |

| | | Comparative | Comparative | Comparative | Comparative | Comparative |
|---|---|---|---|---|---|---|
| | | Example 3 | Example 4 | Example 5 | Example 6 | Example 7 |
| Radiation-sensitive resin | | CJ-3 | CJ-4 | CJ-5 | CJ-6 | CJ-7 |
| composition | | | | | | |
| [A] Resin | Type | A-1 | A-1 | A-1 | A-1 | A-1 |
| | Amount | 100 | 100 | 100 | 100 | 100 |
| | (part(s) by | | | | | |
| | mass) | | | | | |
| [B] | Type | bb-3 | bb-4 | bb-5 | bb-6 | bb-7 |
| Radiation- | Amount | 10.0 | 10.0 | 10.0 | 10.0 | 10.0 |
| sensitive | (part(s) by | | | | | |
| Acid | mass) | | | | | |
| Generater | | | | | | |
| [C] Acid- | Type | C-1 | C-1 | C-1 | C-1 | bb-4 |
| diffusion | Amount | 8.0 | 8.0 | 8.0 | 8.0 | 8.0 |
| Inhibitor | (part(s) by | | | | | |
| | mass) | | | | | |
| [F] High- | Type | F-1 | F-1 | F-1 | F-1 | F-1 |
| Fluorine- | Amount | 3.0 | 3.0 | 3.0 | 3.0 | 3.0 |
| Content- | (part(s) by | | | | | |
| Resin | mass) | | | | | |
| [E] Solvent | Type | E-1/E-2/E-3 | E-1/E-2/E-3 | E-1/E-2/E-3 | E-1/E-2/E-3 | E-1/E-2/E-3 |
| | Amount | 2240/960/30 | 2240/960/30 | 2240/960/30 | 2240/960/30 | 2240/960/30 |
| | (part(s) by | | | | | |
| | mass) | | | | | |

<Formation of Resist Pattern using Positive-Type Radiation-Sensitive Resin Composition for ArF Liquid-Immersion Exposure>

On a 12-inch silicon wafer, a composition for forming an underlayer film (“ARC66”, Brewer Science, Inc.) was applied by using a spin coater (“CLEAN TRACK ACT12”, Tokyo Electron Ltd.), and then heating the composition at 205° C. for 60 seconds to form an underlayer film with 100 nm in average thickness. On this underlayer film, the positive-type radiation-sensitive resin composition for ArF liquid-immersion exposure prepared as above was applied by using the above spin coater, and pre-baking (PB) was performed at 100° C. for 60 seconds. Thereafter, the film was cooled at 23° C. for 30 seconds to form a resist film with 90 nm in average thickness. Then, the resist film was exposed by using an ArF excimer laser liquid-immersion exposure apparatus (“TWINSCAN XT-1900i”, ASML Holding N.V.) under an optical condition of NA=1.35, Dipole (σ=0.9/0.7) through a mask pattern with 40-nm line-and-space. After the exposure, post exposure baking (PEB) was performed at 100° C. for 60 seconds. Thereafter, the resist film was subjected to alkali development by using a TMAH aqueous solution with 2.38 mass % as an alkali developer liquid, washed with water after the development, and further dried to form a positive-type resist pattern (55-nm line-and-space pattern).

### <Evaluation>

As for the resist pattern formed by using the positive-type radiation-sensitive resin composition for ArF liquid-immersion exposure, sensitivity, LWR performance, pattern rectangularity, and a number of development defects were evaluated according to the following methods. The following Table 6 and Table 7 show the results. For measuring a length of the resist pattern, a scanning electron microscope (“CG-5000”, Hitachi High-Tech Corporation) was used.

### [Sensitivity]

In forming the resist pattern using the positive-type radiation-sensitive resin composition for ArF liquid-immersion exposure, an exposure dose for forming the 55-nm line-and-space pattern was specified as an optimal exposure dose, and this optimal exposure dose was specified as sensitivity (mJ/cm²). A case where the sensitivity was 25 mJ/cm² or less was evaluated as “Good”, and a case where the sensitivity was more than 25 mJ/cm² was evaluated as “Poor”.

### [LWR Performance]

The irradiation was performed at the optimal exposure dose determined in the sensitivity evaluation to form a resist pattern with 55-nm line-and-space. The formed resist pattern was observed from above by using the above scanning electron microscope. Variation of the line width was measured at 500 points in total, the three-sigma value was determined from distribution of the measurement values, and this three-sigma value was specified as LWR (nm). A lower LWR value indicates smaller and better line roughness. A case where the LWR value was 2.5 nm or less was evaluated as “Good” LWR performance, and a case where the LWR value was more than 2.5 nm was evaluated as “Poor” LWR performance.

### [Pattern Rectangularity]

A resist pattern with 55-nm line-and-space formed by irradiation at the optimal exposure dose determined in the sensitivity evaluation was observed by using the above scanning electron microscope, and a sectional shape of the line-and-space pattern was evaluated. As for rectangularity of the resist pattern, a case where a ratio of a length of a lower side relative to a length of an upper side in the sectional shape was 1.00 or more and 1.05 or less was evaluated as “A” (extremely good), a case where the ratio was more than 1.05 and 1.10 or less was “B” (Good), and a case where the ratio was more than 1.10 was evaluated as “C” (Poor).

### [Number of Development Defects]

The resist film was exposed at the optimal exposure dose to form a line-and-space pattern with 55 nm in line width to be used as a wafer for defect inspection. A number of defects on this wafer for defect inspection was measured by using a defect inspection apparatus (“KLA2810”, KLA-Tencor Corporation.). Defects with 50 μm or less in diameter was judged as those derived from the resist film, and a number thereof was calculated. As for the number of defects after development, a case where this number of defects judged as those derived from the resist film was 50 or less was evaluated as “Good”, and a case where the number was more than 50 was evaluated as “Poor”.

TABLE 6

| | | | | | Number |
|---|---|---|---|---|---|
| | Radiation- | | | | of |
| | sensitive | | | | Develop- |
| | resin | Sensitivity | LWR | Pattern | ment |
| | composition | (mJ/cm²) | (nm) | Rectangularity | Defects |
| Example 1 | J-1 | 20 | 2.0 | A | 3 |
| Example 2 | J-2 | 21 | 2.1 | A | 6 |
| Example 3 | J-3 | 23 | 2.2 | A | 0 |
| Example 4 | J-4 | 22 | 2.0 | A | 4 |
| Example 5 | J-5 | 21 | 1.9 | A | 2 |
| Example 6 | J-6 | 19 | 2.0 | A | 5 |
| Example 7 | J-7 | 20 | 2.1 | A | 3 |
| Example 8 | J-8 | 21 | 2.2 | A | 8 |
| Example 9 | J-9 | 20 | 2.3 | A | 0 |
| Example 10 | J-10 | 21 | 2.1 | A | 5 |
| Example 11 | J-11 | 20 | 2.1 | A | 3 |
| Example 12 | J-12 | 19 | 1.9 | A | 2 |
| Example 13 | J-13 | 21 | 2.0 | A | 1 |
| Example 14 | J-14 | 23 | 1.8 | A | 2 |
| Example 15 | J-15 | 19 | 2.0 | A | 6 |
| Example 16 | J-16 | 21 | 2.2 | A | 9 |
| Example 17 | J-17 | 23 | 2.1 | A | 4 |
| Example 18 | J-18 | 21 | 2.1 | A | 1 |
| Example 19 | J-19 | 19 | 2.2 | A | 0 |
| Example 20 | J-20 | 18 | 1.8 | A | 8 |
| Example 21 | J-21 | 21 | 1.9 | A | 3 |
| Example 22 | J-22 | 20 | 1.9 | A | 2 |
| Example 23 | J-23 | 20 | 2.0 | A | 7 |
| Example 24 | J-24 | 22 | 2.0 | A | 8 |
| Example 25 | J-25 | 22 | 2.1 | A | 7 |
| Example 26 | J-26 | 21 | 2.0 | A | 3 |
| Example 27 | J-27 | 23 | 2.2 | A | 7 |
| Example 28 | J-28 | 18 | 2.3 | A | 4 |
| Example 29 | J-29 | 22 | 2.1 | A | 18 |

TABLE 7

| | | | | | Number |
|---|---|---|---|---|---|
| | Radiation- | | | | of |
| | sensitive | | | | Develop- |
| | resin | Sensitivity | LWR | Pattern | ment |
| | composition | (mJ/cm²) | (nm) | Rectangularity | Defects |
| Example 30 | J-30 | 21 | 2.3 | A | 20 |
| Example 31 | J-31 | 20 | 2.1 | A | 12 |
| Example 32 | J-32 | 21 | 2.1 | A | 27 |
| Example 33 | J-33 | 23 | 2.2 | A | 22 |
| Example 34 | J-34 | 22 | 2.1 | A | 13 |
| Example 35 | J-35 | 23 | 2.4 | A | 24 |
| Example 36 | J-36 | 21 | 2.4 | A | 20 |
| Example 37 | J-37 | 19 | 1.9 | A | 5 |
| Example 38 | J-38 | 17 | 2.3 | A | 4 |
| Example 39 | J-39 | 22 | 2.0 | A | 5 |
| Example 40 | J-40 | 17 | 2.2 | A | 3 |
| Example 41 | J-41 | 23 | 2.3 | A | 6 |
| Example 42 | J-42 | 23 | 2.2 | A | 4 |
| Example 43 | J-43 | 23 | 2.3 | A | 6 |
| Example 44 | J-44 | 23 | 2.2 | A | 3 |
| Example 45 | J-45 | 17 | 2.1 | A | 3 |
| Example 46 | J-46 | 21 | 2.2 | A | 2 |
| Example 47 | J-47 | 22 | 2.1 | A | 5 |
| Example 48 | J-48 | 21 | 1.9 | A | 8 |
| Example 49 | J-49 | 20 | 2.0 | A | 3 |
| Example 50 | J-50 | 20 | 2.1 | A | 4 |
| Example 51 | J-51 | 20 | 2.0 | A | 3 |
| Comparative | CJ-1 | 30 | 2.8 | B | 149 |
| Example 1 | | | | | |
| Comparative | CJ-2 | 27 | 3.4 | C | 89 |
| Example 2 | | | | | |
| Comparative | CJ-3 | 27 | 3.1 | C | 92 |
| Example 3 | | | | | |
| Comparative | CJ-4 | 34 | 3.2 | B | 232 |
| Example 4 | | | | | |
| Comparative | CJ-5 | 30 | 2.8 | C | 81 |
| Example 5 | | | | | |
| Comparative | CJ-6 | 35 | 3.0 | C | 99 |
| Example 6 | | | | | |
| Comparative | CJ-7 | 30 | 3.2 | C | 82 |
| Example 7 | | | | | |

As obvious from the results of Table 6 and Table 7, the radiation-sensitive resin compositions of Examples 1 to 51 exhibited good results of all the sensitivity, LWR performance, pattern rectangularity, and development defect inhibiting performance, which achieved balance between the properties when used for forming a resist pattern with ArF liquid-immersion exposure. In contrast, the radiation-sensitive resin compositions of Comparative Examples 1 to 7 exhibited poor properties compared with Examples. Therefore, it can be said that the radiation-sensitive resin composition of Examples 1 to 51 can form a resist pattern with good LWR performance and pattern rectangularity and reduced development defects while exhibiting high sensitivity when used for ArF liquid-immersion exposure.

### <Preparation of Positive-Type Radiation-Sensitive Resin Composition for Extreme Ultraviolet Ray (EUV) Exposure>

### Example 52

Mixing 100 parts by mass of (A-14) as the resin [A], 40.0 parts by mass of (B-1) as the radiation-sensitive acid generator [B], 30.0 parts by mass of (C-1) as the acid-diffusion inhibitor [C], 6.0 parts by mass (solid content) of (F-5) as the high-fluorine-content resin [F], and 6,110 parts by mass of a mixed solvent of (E-1)/(E-4) (4,280/1,830 (parts by mass)) as the solvent [E] was performed, and the mixture was filtered with a membrane filter having a pore diameter of 0.2 μm to prepare a radiation-sensitive resin composition (J-52).

### Examples 53 to 72 and Comparative Examples 8 to 12

Radiation-sensitive resin compositions (J-53) to (J-72) and (CJ-8) to (CJ-12) were prepared in the same manner as in Example 52 except that components with types and contents described in the following Table 8 were used.

TABLE 8

| | | Example | Example | Example | Example | Example | Example |
|---|---|---|---|---|---|---|---|
| | | 52 | 53 | 54 | 55 | 56 | 57 |
| Radiation-sensitive resin | | J-52 | J-53 | J-54 | J-55 | J-56 | J-57 |
| composition | | | | | | | |
| [A] Resin | Type | A-14 | A-15 | A-16 | A-17 | A-18 | A-14 |
| | Amount | 100 | 100 | 100 | 100 | 100 | 100 |
| | (part(s) by | | | | | | |
| | mass) | | | | | | |
| [B] Radiation- | Type | B-1 | B-1 | B-1 | B-1 | B-1 | B-3 |
| sensitive Acid | Amount | 40.0 | 40.0 | 40.0 | 40.0 | 40.0 | 40.0 |
| Generater | (part(s) by | | | | | | |
| | mass) | | | | | | |
| [C] Acid- | Type | C-1 | C-1 | C-1 | C-1 | C-1 | C-1 |
| diffusion | Amount | 30.0 | 30.0 | 30.0 | 30.0 | 30.0 | 30.0 |
| Inhibitor | (part(s) by | | | | | | |
| | mass) | | | | | | |
| [F] High- | Type | F-5 | F-5 | F-5 | F-5 | F-5 | F-5 |
| Fluorine- | Amount | 6.0 | 6.0 | 6.0 | 6.0 | 6.0 | 6.0 |
| Content- | (part(s) by | | | | | | |
| Resin | mass) | | | | | | |
| [E] Solvent | Type | E-1/E-4 | E-1/E-4 | E-1/E-4 | E-1/E-4 | E-1/E-4 | E-1/E-4 |
| | Amount | 4280/1830 | 4280/1830 | 4280/1830 | 4280/1830 | 4280/1830 | 4280/1830 |
| | (part(s) by | | | | | | |
| | mass) | | | | | | |
| | | Example | Example | Example | Example | Example | Example |
| | | 58 | 59 | 60 | 61 | 62 | 63 |
| Radiation-sensitive resin | | J-58 | J-59 | J-60 | J-61 | J-62 | J-63 |
| composition | | | | | | | |
| [A] Resin | Type | A-14 | A-14 | A-14 | A-14 | A-14 | A-14 |
| | Amount | 100 | 100 | 100 | 100 | 100 | 100 |
| | (part(s) by | | | | | | |
| | mass) | | | | | | |
| [B] Radiation- | Type | B-4 | B-5 | B-6 | B-11 | B-16 | B-17 |
| sensitive Acid | Amount | 40.0 | 40.0 | 40.0 | 40.0 | 40.0 | 40.0 |
| Generater | (part(s) by | | | | | | |
| | mass) | | | | | | |
| [C] Acid- | Type | C-1 | C-1 | C-1 | C-1 | C-1 | C-1 |
| diffusion | Amount | 30.0 | 30.0 | 30.0 | 30.0 | 30.0 | 30.0 |
| Inhibitor | (part(s) by | | | | | | |
| | mass) | | | | | | |
| [F] High- | Type | F-5 | F-5 | F-5 | F-5 | F-5 | F-5 |
| Fluorine- | Amount | 6.0 | 6.0 | 6.0 | 6.0 | 6.0 | 6.0 |
| Content- | (part(s) by | | | | | | |
| Resin | mass) | | | | | | |
| [E] Solvent | Type | E-1/E-4 | E-1/E-4 | E-1/E-4 | E-1/E-4 | E-1/E-4 | E-1/E-4 |
| | Amount | 4280/1830 | 4280/1830 | 4280/1830 | 4280/1830 | 4280/1830 | 4280/1830 |
| | (part(s) by | | | | | | |
| | mass) | | | | | | |
| | | Example | Example | Example | Example | Example | Example |
| | | 64 | 65 | 66 | 67 | 68 | 69 |
| Radiation-sensitive resin | | J-64 | J-65 | J-66 | J-67 | J-68 | J-69 |
| composition | | | | | | | |
| [A] Resin | Type | A-14 | A-14 | A-14 | A-14 | A-14 | A-14 |
| | Amount | 100 | 100 | 100 | 100 | 100 | 100 |
| | (part(s) by | | | | | | |
| | mass) | | | | | | |
| [B] Radiation- | Type | B-19 | B-20 | B-21 | B-1 | B-1 | B-1 |
| sensitive Acid | Amount | 40.0 | 40.0 | 40.0 | 40.0 | 40.0 | 40.0 |
| Generater | (part(s) by | | | | | | |
| | mass) | | | | | | |
| [C] Acid- | Type | C-1 | C-1 | C-1 | C-2 | C-4 | C-6 |
| diffusion | Amount | 30.0 | 30.0 | 30.0 | 30.0 | 30.0 | 30.0 |
| Inhibitor | (part(s) by | | | | | | |
| | mass) | | | | | | |
| [F] High- | Type | F-5 | F-5 | F-5 | F-5 | F-5 | F-5 |
| Fluorine- | Amount | 6.0 | 6.0 | 6.0 | 6.0 | 6.0 | 6.0 |
| Content- | (part(s) by | | | | | | |
| Resin | mass) | | | | | | |
| [E] Solvent | Type | E-1/E-4 | E-1/E-4 | E-1/E-4 | E-1/E-4 | E-1/E-4 | E-1/E-4 |
| | Amount | 4280/1830 | 4280/1830 | 4280/1830 | 4280/1830 | 4280/1830 | 4280/1830 |
| | (part(s) by | | | | | | |
| | mass) | | | | | | |
| | | | | | Comparative | Comparative | Comparative |
| | | Example | Example | Example | Example | Example | Example |
| | | 70 | 71 | 72 | 8 | 9 | 10 |
| Radiation-sensitive resin | | J-70 | J-71 | J-72 | CJ-8 | CJ-9 | CJ-10 |
| composition | | | | | | | |
| [A] Resin | Type | A-14 | A-14 | A-14 | A-14 | A-14 | A-14 |
| | Amount | 100 | 100 | 100 | 100 | 100 | 100 |
| | (part(s) by | | | | | | |
| | mass) | | | | | | |
| [B] Radiation- | Type | B-1/B-16 | B-1/B-17 | B-1/B-19 | bb-2 | bb-3 | bb-5 |
| sensitive | Amount | 20.0/20.0 | 20.0/20.0 | 20.0/20.0 | 40.0 | 40.0 | 40.0 |
| Acid | (part(s) by | | | | | | |
| Generater | mass) | | | | | | |
| [C] Acid- | Type | C-1 | C-1 | C-1 | C-1 | C-1 | C-1 |
| diffusion | Amount | 30.0 | 30.0 | 30.0 | 30.0 | 30.0 | 30.0 |
| Inhibitor | (part(s) by | | | | | | |
| | mass) | | | | | | |
| [F] High- | Type | F-5 | F-5 | F-5 | F-5 | F-5 | F-5 |
| Fluorine- | Amount | 6.0 | 6.0 | 6.0 | 6.0 | 6.0 | 6.0 |
| Content- | (part(s) by | | | | | | |
| Resin | mass) | | | | | | |
| [E] Solvent | Type | E-1/E-4 | E-1/E-4 | E-1/E-4 | E-1/E-4 | E-1/E-4 | E-1/E-4 |
| | Amount | 4280/1830 | 4280/1830 | 4280/1830 | 4280/1830 | 4280/1830 | 4280/1830 |
| | (part(s) by | | | | | | |
| | mass) | | | | | | |

| | | Comparative | Comparative |
|---|---|---|---|
| | | Example 11 | Example 12 |
| Radiation-sensitive resin | | CJ-11 | CJ-12 |
| composition | | | |
| [A] Resin | Type | A-14 | A-14 |
| | Amount | 100 | 100 |
| | (part(s) by | | |
| | mass) | | |
| [B] Radiation- | Type | bb-6 | bb-7 |
| sensitive Acid | Amount | 40.0 | 40.0 |
| Generater | (part(s) by | | |
| | mass) | | |
| [C] Acid- | Type | C-1 | C-1 |
| diffusion | Amount | 30.0 | 30.0 |
| Inhibitor | (part(s) by | | |
| | mass) | | |
| [F] High- | Type | F-5 | F-5 |
| Fluorine- | Amount | 6.0 | 6.0 |
| Content- | (part(s) by | | |
| Resin | mass) | | |
| [E] Solvent | Type | E-1/E-4 | E-1/E-4 |
| | Amount | 4280/1830 | 4280/1830 |
| | (part(s) by | | |
| | mass) | | |

<Formation of Resist Pattern using Positive-Type Radiation-Sensitive Resin Composition for EUV Exposure>

On a 12-inch silicon wafer, a composition for forming an underlayer film (“ARC66”, Brewer Science, Inc.) was applied by using a spin coater (“CLEAN TRACK ACT12”, Tokyo Electron Ltd.), and then heating the composition at 205° C. for 60 seconds to form an underlayer film with 105 nm in average thickness. On this underlayer film, the radiation-sensitive resin composition for EUV exposure prepared as above was applied by using the above spin coater, and PB was performed at 130° C. for 60 seconds. Thereafter, the film was cooled at 23° C. for 30 seconds to form a resist film with 55 nm in average thickness. Then, the resist film was exposed by using an EUV exposure apparatus (“NXE 3300”, ASML Holding N.V.) with NA=0.33, illumination condition: Conventional s=0.89, mask: imec DEFECT32FFR02. After the exposure, PEB was performed at 120° C. for 60 seconds. Thereafter, the resist film was subjected to alkali development by using a TMAH aqueous solution with 2.38 mass % as an alkali developer liquid, washed with water after the development, and further dried to form a positive-type resist pattern (25-nm line-and-space pattern).

### <Evaluation>

As for the resist pattern formed by using the positive-type radiation-sensitive resin composition for EUV exposure, sensitivity, LWR performance, pattern rectangularity, and a number of development defects were evaluated according to the following methods. The following Table 9 shows the results. For measuring a length of the resist pattern, a scanning electron microscope (“CG-5000”, Hitachi High-Tech Corporation) was used.

### [Sensitivity]

In forming the resist pattern using the positive-type radiation-sensitive resin composition for EUV exposure, an exposure dose for forming the 25-nm line-and-space pattern was specified as an optimal exposure dose, and this optimal exposure dose was specified as sensitivity (mJ/cm²). A case where the sensitivity was 40 mJ/cm² or less was evaluated as “Good”, and a case where the sensitivity was more than 40 mJ/cm² was evaluated as “Poor”.

### [LWR Performance]

The irradiation was performed at the optimal exposure dose determined in the sensitivity evaluation to form a resist pattern by regulating a mask size so as to form a 25-nm line-and-space pattern. The formed resist pattern was observed from above by using the above scanning electron microscope. Variation of the line width was measured at 500 points in total, the three-sigma value was determined from distribution of the measurement values, and this three-sigma value was specified as LWR (nm). A lower LWR value indicates smaller and better line roughness. A case where the LWR value was 3.0 nm or less was evaluated as “Good” LWR performance, and a case where the LWR value was more than 3.0 nm was evaluated as “Poor” LWR performance.

### [Pattern Rectangularity]

A resist pattern with 32-nm line-and-space formed by irradiation at the optimal exposure dose determined in the sensitivity evaluation was observed by using the above scanning electron microscope, and a sectional shape of the line-and-space pattern was evaluated. As for rectangularity of the resist pattern, a case where a ratio of a length of a lower side relative to a length of an upper side in the sectional shape was 1.00 or more and 1.05 or less was evaluated as “A” (extremely good), a case where the ratio was more than 1.05 and 1.10 or less was “B” (Good), and a case where the ratio was more than 1.10 was evaluated as “C” (Poor).

### [Number of Development Defects]

The resist film was exposed at the optimal exposure dose to form a line-and-space pattern with 25 nm in line width to be used as a wafer for defect inspection. A number of defects on this wafer for defect inspection was measured by using a defect inspection apparatus (“KLA2810”, KLA-Tencor Corporation.). Defects with 50 μm or less in diameter was judged as those derived from the resist film, and a number thereof was calculated. As for the number of defects after development, a case where this number of defects judged as those derived from the resist film was 50 or less was evaluated as “Good”, and a case where the number was more than 50 was evaluated as “Poor”.

TABLE 9

| | Radiation- | | | | Number of |
|---|---|---|---|---|---|
| | sensitive | | | | Develop- |
| | resin | Sensitivity | LWR | Pattern | ment |
| | composition | (mJ/cm²) | (nm) | Rectangularity | Defects |
| Example 52 | J-52 | 35 | 2.6 | A | 23 |
| Example 53 | J-53 | 34 | 2.7 | A | 25 |
| Example 54 | J-54 | 36 | 2.5 | A | 32 |
| Example 55 | J-55 | 35 | 2.5 | A | 22 |
| Example 56 | J-56 | 32 | 2.4 | A | 14 |
| Example 57 | J-57 | 33 | 2.7 | A | 21 |
| Example 58 | J-58 | 35 | 2.3 | A | 12 |
| Example 59 | J-59 | 35 | 2.5 | A | 4 |
| Example 60 | J-60 | 35 | 2.0 | A | 21 |
| Example 61 | J-61 | 36 | 2.4 | A | 21 |
| Example 62 | J-62 | 32 | 2.3 | A | 15 |
| Example 63 | J-63 | 35 | 2.5 | A | 18 |
| Example 64 | J-64 | 32 | 2.3 | A | 32 |
| Example 65 | J-65 | 33 | 2.5 | A | 14 |
| Example 66 | J-66 | 32 | 2.3 | A | 12 |
| Example 67 | J-67 | 34 | 2.3 | A | 7 |
| Example 68 | J-68 | 35 | 2.5 | A | 21 |
| Example 69 | J-69 | 33 | 2.6 | A | 28 |
| Example 70 | J-70 | 32 | 2.3 | A | 27 |
| Example 71 | J-71 | 34 | 2.4 | A | 19 |
| Example 72 | J-72 | 32 | 2.4 | A | 25 |
| Comparative | CJ-8 | 40 | 3.5 | C | 213 |
| Example 8 | | | | | |
| Comparative | CJ-9 | 42 | 3.6 | C | 245 |
| Example 9 | | | | | |
| Comparative | CJ-10 | 43 | 3.4 | C | 328 |
| Example 10 | | | | | |
| Comparative | CJ-11 | 42 | 3.6 | C | 189 |
| Example 11 | | | | | |
| Comparative | CJ-12 | 45 | 3.5 | C | 260 |
| Example 12 | | | | | |

As obvious from the results of Table 9, the radiation-sensitive resin compositions of Examples 52 to 72 exhibited good results of all the sensitivity, LWR performance, pattern rectangularity, and development defect inhibiting performance when used for forming a resist pattern with EUV exposure. In contrast, the radiation-sensitive resin compositions of Comparative Examples 8 to 12 exhibited poor properties compared with Examples.

<Preparation of Negative-Type Radiation-Sensitive Resin Composition for ArF Liquid-Immersion Exposure, and Formation and Evaluation of Resist Pattern Using this Composition>

### Example 73

Mixing 100 parts by mass of (A-1) as the resin [A], 12.0 parts by mass of (B-1) as the radiation-sensitive acid generator [B], 6.0 parts by mass of (C-4) as the acid-diffusion inhibitor [C], 3.0 parts by mass (solid content) of (F-4) as the high-fluorine-content resin [F], and 3,230 parts by mass of a mixed solvent of (E-1)/(E-2)/(E-3) (2,240/960/30 (parts by mass)) as the solvent [E] was performed, and the mixture was filtered with a membrane filter having a pore diameter of 0.2 μm to prepare a radiation-sensitive resin composition (J-73).

On a 12-inch silicon wafer, a composition for forming an underlayer film (“ARC66”, Brewer Science, Inc.) was applied by using a spin coater (“CLEAN TRACK ACT12”, Tokyo Electron Ltd.), and then heating the composition at 205° C. for 60 seconds to form an underlayer film with 100 nm in average thickness. On this underlayer film, the radiation-sensitive resin composition (J-73) prepared as above was applied by using the above spin coater, and pre-baking (PB) was performed at 100° C. for 60 seconds. Thereafter, the film was cooled at 23° C. for 30 seconds to form a resist film with 90 nm in average thickness. Then, the resist film was exposed by using an ArF excimer laser liquid-immersion exposure apparatus (“TWINSCAN XT-1900i”, ASML Holding N.V.) under an optical condition of NA=1.35, Annular (6=0.8/0.6) through a mask pattern with 40-nm hole and 105-nm pitch. After the exposure, post exposure baking (PEB) was performed at 100° C. for 60 seconds. Thereafter, the resist film was subjected to organic solvent development by using n-butyl acetate as an organic solvent developer liquid, and dried to form a negative-type resist pattern (60-nm hole and 120-nm pitch).

As for the resist pattern formed by using the negative-type radiation-sensitive resin composition for ArF liquid-immersion exposure, CDU performance was evaluated according to the following method. For measuring a length of the resist pattern, a scanning electron microscope (“CG-5000”, Hitachi High-Tech Corporation) was used.

### [CDU Performance]

The resist pattern with 60-nm hole and 120-nm pitch was subjected to length measurement from above the pattern at given 1,800 points in total by using the above scanning electron microscope. The variation of size (36) was determined to specify this value as CDU performance (nm). A smaller value of CDU indicates smaller and better variation of the hole diameter with a long period.

The resist pattern using the radiation-sensitive resin composition (J-73) was evaluated as noted above, and as a result, the radiation-sensitive resin composition containing the polymer (A) and the compound (B) was found to exhibit good CDU performance when forming the negative-type resist pattern with ArF liquid-immersion exposure.

<Preparation of Negative-Type Radiation-Sensitive Resin Composition for EUV Exposure, and Formation and Evaluation of Resist Pattern Using this Composition>

### Example 74

Mixing 100 parts by mass of (A-17) as the resin [A], 40.0 parts by mass of (B-6) as the radiation-sensitive acid generator [B], 40.0 parts by mass of (C-2) as the acid-diffusion inhibitor [C], 3.0 parts by mass (solid content) of (F-5) as the high-fluorine-content resin [F], and 6,110 parts by mass of a mixed solvent of (E-1)/(E-4) (4,280/1,830 (parts by mass)) as the solvent [E] was performed, and the mixture was filtered with a membrane filter having a pore diameter of 0.2 μm to prepare a radiation-sensitive resin composition (J-74).

On a 12-inch silicon wafer, a composition for forming an underlayer film (“ARC66”, Brewer Science, Inc.) was applied by using a spin coater (“CLEAN TRACK ACT12”, Tokyo Electron Ltd.), and then heating the composition at 205° C. for 60 seconds to form an underlayer film with 105 nm in average thickness. On this underlayer film, the radiation-sensitive resin composition (J-74) prepared as above was applied by using the above spin coater, and PB was performed at 130° C. for 60 seconds. Thereafter, the film was cooled at 23° C. for 30 seconds to form a resist film with 55 nm in average thickness. Then, the resist film was exposed by using an EUV exposure apparatus (“NXE 3300”, ASML Holding N.V.) with NA=0.33, illumination condition: Conventional s=0.89, mask: imec DEFECT32FFR02. After the exposure, PEB was performed at 120° C. for 60 seconds. Thereafter, the resist film was subjected to organic solvent development by using n-butyl acetate as an organic solvent developer liquid, and dried to form a negative-type resist pattern (30-nm hole and 60-nm pitch).

CDU performance of the resist pattern using the radiation-sensitive resin composition (J-74) was evaluated similarly to the evaluation of the resist pattern using the negative-type radiation-sensitive resin composition for ArF liquid-immersion exposure. As a result, the radiation-sensitive resin composition containing the polymer (A) and the compound (B) was found to exhibit good CDU performance also when forming the negative-type resist pattern with EUV exposure.

The radiation-sensitive resin composition and the method for forming a resist pattern as described above exhibit good sensitivity to exposure light, and excellent LWR performance, pattern rectangularity, and development defect inhibiting performance. Therefore, these can be suitably used for processing process of a semiconductor device etc., which is forecasted to advance further miniaturization for the future.

Obviously, numerous modifications and variations of the present invention(s) are possible in light of the above teachings. It is therefore to be understood that within the scope of the appended claims, the invention(s) may be practiced otherwise than as specifically described herein.

What is claimed is:

1. A radiation-sensitive composition comprising:
a polymer comprising an acid-releasable group; and
a compound represented by formula (1): 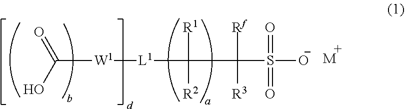
wherein L¹ represents a group comprising a (thio)acetal ring formed by replacing each of two methylene groups of a monocyclic saturated aliphatic hydrocarbon ring by a (thio)ether bond so that two oxygens, two sulfurs, or one oxygen and one sulfur are bonded to one and the same carbon, or represents a group represented by the following formula (L-2): 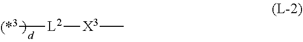
wherein L² represents a bridged alicyclic group having 7 or more carbon atoms; X³ represents a single bond, an oxygen atom, a sulfur atom, or —SO₂—; d represents 1 or 2; and *³ represents a chemical bond to W¹ or a carboxy group,
W¹ represents a single bond or a (b+1)-valent organic group having 1 to 40 carbon atoms; R¹, R², and R³ each independently represent a hydrogen atom, a hydrocarbon group having 1 to 10 carbon atoms, a fluorine atom, or a fluoroalkyl group; R^{f} represents a fluorine atom or a fluoroalkyl group; a represents an integer of 0 to 8; b represents an integer of 1 to 4; d represents 1 or 2; when L¹ represents the group represented by the formula (L-2), d in the formula (1) and d in the formula (L-2) are the same values; when a represents 2 or more, a plurality of R¹ are the same or different, and a plurality of R² are the same or different; when d represents 2, a plurality of W¹ are the same or different, and a plurality of b are the same or different; and M⁺ represents a monovalent cation.

2. The radiation-sensitive composition according to claim 1, wherein the L¹ represents a group comprising a (thio)acetal ring, and is represented by formula (L-1): 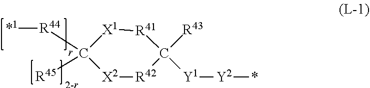
wherein X¹ and X² each independently represent an oxygen atom or a sulfur atom; R⁴¹ represents a single bond or an alkanediyl group having 1 to 10 carbon atoms; r represents 1 or 2; when r represents 1, R⁴⁴ represents a single bond and R⁴⁵ represents a hydrogen atom or a monovalent hydrocarbon group having 1 to 10 carbon atoms, or R⁴⁴ and R⁴⁵ taken together represent a cyclic structure forming a spiro-ring structure together with the carbon atom to which R⁴⁴ and R⁴⁵ are bonded and the (thio)acetal ring in the formula (L-1); when r represents 2, R⁴⁴ represents a single bond; among partial structures —W¹—(COOH)_{b} bonded to L¹ in the formula (1), b in a partial structure in which W¹ represents a single bond represents 1; R⁴², R⁴³, Y¹, and Y² satisfy the following (i), (ii), or (iii):
(i) R⁴² represents an alkanediyl group having 1 to 10 carbon atoms, R⁴³ represents a hydrogen atom or a monovalent hydrocarbon group having 1 to 10 carbon atoms, Y¹ represents a single bond or a divalent linking group, and Y² represents a single bond;
(ii) R⁴² and Y¹ taken together represent a cyclic structure forming a condensed cyclic structure together with the carbon atom to which R⁴² and Y¹ are bonded and the (thio)acetal ring in the formula (L-1), R⁴³ represents a hydrogen atom or a monovalent hydrocarbon group having 1 to 10 carbon atoms, and Y² represents a single bond or a divalent linking group;
(iii) R⁴³ and Y¹ taken together represent a cyclic structure forming a spiro-ring structure together with the carbon atom to which R⁴³ and Y¹ are bonded and the (thio)acetal ring in the formula (L-1), R⁴² represents an alkanediyl group having 1 to 10 carbon atoms, and Y² represents a single bond or a divalent linking group; and
*¹ represents a chemical bond to W¹ or the carboxy group in the formula (1), and * represents a chemical bond.

3. The radiation-sensitive composition according to claim 1, wherein one or more partial structures —W¹—(COOH)_{b} bonded to L¹ in the formula (1) are a group in which W¹ comprises a cyclic structure, and one or more carboxy groups are bonded to the ring in W¹.

4. The radiation-sensitive composition according to claim 3, wherein one or more carboxy groups are bonded to an aromatic ring.

5. The radiation-sensitive composition according to claim 1, wherein
in one or more partial structures —W¹—(COOH)_{b} bonded to L¹ in the formula (1), W¹ represents a single bond,
L¹ in the formula (1) represents a group comprising a (thio)acetal ring, and comprises a ring R^{x} forming a condensed cyclic structure or a spiro-ring structure together with the (thio)acetal ring in L¹, and
a carboxy group is bonded to the ring R^{X} or the thio(acetal) ring.

6. The radiation-sensitive composition according to claim 5, wherein
the ring R^{x} represents a polycyclic aliphatic hydrocarbon ring, a polycyclic saturated heteroring, or a polycyclic aromatic hydrocarbon ring, and
a carboxy group is bonded to the polycyclic aliphatic hydrocarbon ring, the polycyclic saturated heteroring, or the polycyclic aromatic hydrocarbon ring in the ring R^{x}.

7. The radiation-sensitive composition according to claim 1, wherein the polymer comprises a structural unit represented by formula (3): 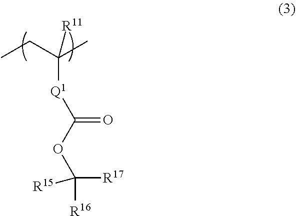
wherein R¹¹ represents a hydrogen atom, a fluorine atom, a methyl group, a trifluoromethyl group, or an alkoxyalkyl group; Q¹ represents a single bond or a substituted or unsubstituted divalent hydrocarbon group; R¹⁵ represents a substituted or unsubstituted monovalent hydrocarbon group having 1 to 8 carbon atoms; R¹⁶ and R¹⁷ each independently represent a monovalent chain hydrocarbon group having 1 to 8 carbon atoms or a monovalent alicyclic hydrocarbon group having 3 to 12 carbon atoms, or R¹⁶ and R¹⁷ taken together represent a divalent alicyclic hydrocarbon group having 3 to 12 carbon atoms together with the carbon atom to which R¹⁶ and R¹⁷ are bonded.

8. The radiation-sensitive composition according to claim 1, further comprising a compound that generates an acid weaker than an acid generated from the compound represented by the formula (1) by irradiation with radiation.

9. A method for forming a resist pattern, comprising:
applying the radiation-sensitive composition according to claim 1 on a substrate to form a resist film;
exposing the resist film; and
developing the exposed resist film.

10. The method for forming a resist pattern according to claim 9, wherein, developing comprises developing the exposed resist film with an alkali developer liquid.

11. A radiation-sensitive acid generator represented by formula (1): 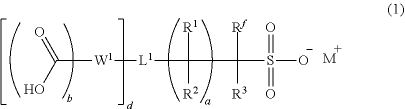
wherein L¹ represents a group comprising a (thio)acetal ring formed by replacing each of two methylene groups of a monocyclic saturated aliphatic hydrocarbon ring by a (thio)ether bond so that two oxygens, two sulfurs, or one oxygen and one sulfur are bonded to one and the same carbon, or represents a group represented by formula (L-2): 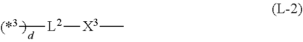
wherein L² represents a bridged alicyclic group having 7 or more carbon atoms; X³ represents a single bond, an oxygen atom, a sulfur atom, or —SO₂—; d represents 1 or 2; and *³ represents a chemical bond to W¹ or a carboxy group,
W¹ represents a single bond or a (b+1)-valent organic group having 1 to 40 carbon atoms; R¹, R², and R³ each independently represent a hydrogen atom, a hydrocarbon group having 1 to 10 carbon atoms, a fluorine atom, or a fluoroalkyl group; R^{f} represents a fluorine atom or a fluoroalkyl group; a represents an integer of 0 to 8; b represents an integer of 1 to 4; d represents 1 or 2; when L¹ represents the group represented by the formula (L-2), d in the formula (1) and d in the formula (L-2) are the same values; when a represents 2 or more, a plurality of R¹ are the same or different, and a plurality of R² are the same or different; when d represents 2, a plurality of W¹ are the same or different, and a plurality of b are the same or different; and M⁺ represents a monovalent cation.
